(19) United States
(12) Patent Application Publication
(10) Pub. No.: US 2009/0054955 A1
(43) Pub. Date: Feb. 26, 2009

(54) Systems and Methods for Treating Neurological Disorders by Light Stimulation

(76) Inventors: Brian H. Kopell, Milwaukee, WI (US); Harry T. Whelan, Whitefish Bay, WI (US); Thomas E. Prieto, Grafton, WI (US); Mark S. Geisler, Franklin, WI (US)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 12/195,257

(22) Filed: Aug. 20, 2008

Related U.S. Application Data
(60) Provisional application No. 60/956,829, filed on Aug. 20, 2007.

(51) Int. Cl. A61N 5/06

(52) U.S. Cl. 607/88

(57) ABSTRACT

Systems and methods for treating neurological disorders by light stimulation are disclosed herein. A brain neuroillumination system includes an implantable housing; a control unit carried by the implantable housing; a neuroillumination delivery device including at least one light emission site; at least one light source operatively coupled to the control unit and optically coupled to the at least one light emission site; and a near infrared spectroscopy unit coupled to the control unit, wherein the near infrared spectroscopy unit is configures to detect an optical signal corresponding to at least one from the group of a cytochrome oxidase state and a hemoglobin oxygenation state.

### RELATED APPLICATIONS

This application claims the benefit of and priority to U.S. Provisional Application Ser. No. 60/956,829, filed on Aug. 20, 2007, the entirety of this application hereby incorporated by reference.

### FIELD

The embodiments disclosed herein relate to systems, apparatus, devices, and methods for providing, applying, or delivering optical signals to neural targets within the brain and/or elsewhere. More particularly, the embodiments disclosed herein relate to illuminating or optically irradiating neural targets in situations in which a neuroprotective effect may be desirable. Additional embodiments are directed towards intraventricular systems and devices for illuminating neural targets.

### BACKGROUND

Neuromodulation is technology that works directly upon nerves by altering or modulating nerve activity by delivering electrical or pharmaceutical agents directly to a target area. Most frequently, people think of neuromodulation in the context of chronic pain relief, the most common indication. However, there are a plethora of neuromodulation applications, such as deep brain stimulation (DBS) treatment for Parkinson disease, sacral nerve stimulation for pelvic disorders and incontinence, and spinal cord stimulation for ischemic disorders (angina, peripheral vascular disease). Electrical stimulation techniques, such as DBS, are used to stimulate nerve structures in specific areas of the brain to either excite or inhibit cell activity by modifying the electrical activity between neurons, therefore only treating the neural activity associated with a particular neurological disorder. These techniques do not typically directly affect the cellular processes within neurons which are often the underlying cause of the neurological disorder itself. As such these electrically based neuromodulation devices often do not affect the course of the neurological disorder but merely the symptoms associated with it.

### SUMMARY

Systems and methods for treating neurological disorders by light stimulation are disclosed herein. According to aspects illustrated herein, there is provided a neuroillumination probe that includes an elongate member; a first light emission site carried by the elongate member; and a second light emission site carried by the elongate member, wherein the first light emission site and the second light emission site are separated along a length of the elongate member by an expected approximate distance between a deep brain neural target and a cortical neural target.

According to aspects illustrated herein, there is provided a neuroillumination probe that includes an elongate member including a proximal segment and a distal segment, the proximal segment having a different geometric shape than the distal segment; and a first light emission site carried by the elongate member, wherein the distal segment has a geometric shape that corresponds to a boundary of an anatomical structure. In an embodiment, the neuroillumination probe further includes a second light emission site carried by the elongate member.

According to aspects illustrated herein, there is provided a brain neuroillumination system that includes an implantable housing; a control unit carried by the implantable housing; a neuroillumination probe including an elongate portion and a contoured portion, the neuroillumination probe having at least a first light emission site; and a light source operatively coupled to the control unit and optically coupled to the first light emission site, the light source carried by one from the group of the implantable housing and the neuroillumination probe, wherein the contoured portion of the neuroillumination probe has a geometric shape that corresponds to a boundary of an anatomical structure. In an embodiment, the brain neuroillumination system further includes a telemetry circuit carried by the implantable housing; and a programming device configured for wireless signal communication with the telemetry unit.

According to aspects illustrated herein, there is provided a brain neuroillumination system that includes an implantable housing; a control unit carried by the implantable housing; a neuroillumination delivery device including at least one light emission site; at least one light source operatively coupled to the control unit and optically coupled to the at least one light emission site; and a near-infrared spectroscopy unit coupled to the control unit, wherein the near-infrared spectroscopy unit is configured to detect an optical signal corresponding to at least one from the group of a cytochrome oxidase state and a hemoglobin oxygenation state.

According to aspects illustrated herein, there is provided an illumination shunt system that includes a shunt catheter; a distal catheter; a fluid reservoir in fluidic communication with the shunt catheter and the distal catheter; a shunt control module; a one-way valve operatively coupled to the shunt control module and in fluidic communication with the shunt catheter; a light source; an implantable illumination probe including at least one light emission site that is optically coupled to the light source; and an illumination control module operatively coupled to the light source. In an embodiment, the illumination shunt system further includes an implantable housing that carries the shunt control module and the illumination control module. In an embodiment, the illumination shunt system further includes a telemetry circuit coupled to the illumination control module; and a programming device configured for wireless communication with the telemetry circuit.

According to aspects illustrated herein, there is provided a neuromodulation method that includes applying electrical signals to a neural target; determining if the electrical signals are applied in a manner that exceeds a predetermined or programmable reference upper electrical stimulation limit; and applying optical signals to at least a portion of the neural target. In an embodiment, the method further comprises determining if the electrical signals are applied in a manner that exceeds an expanded upper electrical stimulation limit. In an embodiment, the method further comprises discontinuing the application of electrical signals in the event that the electrical signals are applied in a manner that exceeds the expanded upper electrical stimulation limit. In an embodiment, the method further includes determining if the electrical signals are applied in a manner that exceeds the reference upper electrical stimulation limit and which is below the expanded upper electrical stimulation limit; and continuing to apply electrical signals to the neural target.

According to aspects illustrated herein, there is provided a neuroillumination method that includes the steps of (a) acquiring a first set of sensed parameters corresponding to a neural target using a first set of sensing devices; (b) acquiring a second set of sensed parameters corresponding to the neural target using a second set of sensing devices; (c) determining whether at least one set of sensed parameters corresponds to at least one from the group of an oxidative stress condition, a neurodegeneration condition, a neuronal damage condition, and an abnormal metabolic condition within the neural target; and (d) applying optical signals to the neural target. In an embodiment, the method further includes determining whether the first set of sensed parameters and the second set of sensed parameters correspond to at least one from the group of an oxidative stress condition, a neurodegeneration condition, a neuronal damage condition and an abnormal metabolic condition within the neural target.

According to aspects illustrated herein, there is provided a neuroillumination method that includes measuring a bodily fluid pressure parameter; determining whether the bodily fluid pressure parameter exceeds a reference fluid pressure value; and applying optical signals to a neural target.

According to aspects illustrated herein, there is provided a method for treating a neural condition of a patient that includes implanting an illumination probe into a cerebral ventricle of the patient, the illumination probe operable to emit near-infrared light; and preferentially directing near-infrared light emitted by the illumination probe toward a predetermined set of neural targets.

According to aspects illustrated herein, there is provided a tissue illumination method that includes implanting an illumination shunt system into a patient, the illumination shunt system including: a ventricular shunt apparatus; a tissue illumination apparatus; and a telemetry circuit coupled to at least one of the ventricular shunt apparatus and the tissue illumination apparatus; establishing a wireless communication link between the telemetry circuit and a programming device; and wirelessly communicating one from the group of a shunt control parameter and a tissue illumination parameter from the programming device to the telemetry circuit. In an embodiment, the method further includes illuminating neural tissue with near-infrared light. In an embodiment, the method further includes preferentially applying illumination to a predetermined set of neural targets, the illumination applied at a wavelength ranging from about 670 nm to about 820 nm.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1A-1L are simplified block diagrams of some of the main components of brain neuromodulation systems (BNS) for illuminating or irradiating a set of neural targets of the presently disclosed embodiments.

FIGS. 1M and 1N are simplified block diagrams of some of the main components of brain neuromodulation systems for applying optical signals in association with the application of electrical signals to one or more neural targets, and/or sensing signals or substances of the presently disclosed embodiments.

FIG. 2A is a cross sectional anatomical illustration of a representative embodiment of the BNS of FIG. 1A, configured for cortical neural target illumination.

FIG. 2B is a cross sectional anatomical illustration of a representative embodiment of the BNS of FIG. 1A, configured for subcortical (e.g., deep brain) neural target illumination.

FIG. 2C is a cross sectional anatomical illustration of a representative embodiment of the BNS of FIG. 1B, configured for cortical neural target illumination.

FIG. 2D is a cross sectional anatomical illustration of a representative embodiment of the BNS of FIG. 1C, configured to provide cortical neuroillumination.

FIG. 2E is a cross sectional anatomical illustration of a representative embodiment of the BNS of FIG. 1C, configured to apply subcortical or deep brain neuroillumination.

FIG. 2F is a cross sectional anatomical illustration of a representative embodiment of the BNS of FIG. 1D, configured for cortical neural target illumination.

FIG. 2G is a cross sectional anatomical illustration of a representative embodiment of the BNS of FIG. 1D, configured to apply optical irradiation to a subcortical neural target.

FIG. 2H is a cross sectional anatomical illustration of the BNS of FIG. 1E configured for cortical neural illumination.

FIG. 2I is a cross sectional anatomical illustration of the BNS of FIG. 1E configured for subcortical neural illumination.

FIG. 2J is a cross sectional anatomical illustration of a representative embodiment of the BNS of FIG. 1G or FIG. 1J, configured to provide neuroillumination to one or more cortical targets.

FIG. 2K is a cross sectional anatomical illustration of a representative embodiment of the BNS of FIG. 1H or FIG. 1K, configured to apply optical signals to a cortical neural target.

FIG. 2L is a cross sectional anatomical illustration of a representative embodiment of the BNS of FIG. 1H or FIG. 1K, configured to apply optical signals to a subcortical neural target.

FIG. 2M is a cross sectional anatomical illustration of a representative embodiment of the BNS of FIG. 1I or FIG. 1L, configured to provide subcortical neuroillumination.

FIG. 2N is a cross sectional anatomical illustration of a representative embodiment of the BNS of FIG. 1J, configured to provide subcortical neuroillumination.

FIG. 2O is a cross sectional anatomical illustration of a representative embodiment of a BNS configured to deliver optical irradiation or illumination to both cortical and subcortical neural targets.

FIG. 2P is a cross sectional anatomical illustration of a representative embodiment of a BNS configured for both neuroillumination and electrical stimulation operations.

FIG. 2Q is a cross sectional anatomical illustration of a representative embodiment of a BNS configured for neuroillumination and sensing operations.

FIG. 3A is a cross sectional anatomical illustration of an embodiment of an intraventricular BNS having a neuroillumination probe implanted into or proximate to the anterior horn of the lateral ventricle.

FIG. 3B is a cross sectional anatomical illustration of an embodiment of an intraventricular BNS having a neuroillumination probe implanted into the third ventricle.

FIG. 3C is a cross sectional anatomical illustration of an embodiment of an intraventricular BNS having a contoured or shaped neuroillumination probe implanted into the third ventricle.

FIG. 3D is a cross sectional anatomical illustration of an embodiment of an intraventricular BNS having a neuroillumination probe into the inferior horn of the lateral ventricle.

FIG. 3E is a cross sectional anatomical illustration of an embodiment of an intraventricular BNS having a neuroillumination probe implanted into along a posterior-anterior span of the lateral ventricle.

FIG. 4A is a cross sectional anatomical illustration of an embodiment of a ventricular illumination and drainage system (VIDS) having an illumination shunt.

FIG. 4B is a schematic illustration of an embodiment of an illumination shunt.

FIG. 5A is an illustration showing a representative periodic pattern of light in which the light source alternates between ON and OFF states at particular time intervals.

FIG. 5B is an illustration showing a representative periodic pattern of light in which the light source alternates between ON and OFF states during particular subintervals of time.

FIG. 6A is an illustration of an embodiment of an end of an optical fiber bundle having a single segment.

FIG. 6B is an illustration of an embodiment of an end of optical fiber bundle divided into two branches.

FIG. 7A is an illustration of an embodiment of an end of an optical fiber bundle with the optical fibers flared to increase an angle over which light is emitted from a tip.

FIG. 7B is an illustration of an embodiment of an end of an optical fiber bundle with a lens attached to or formed from a tip region to control an angle over which light is emitted.

FIG. 7C is an illustration of an embodiment of an optical fiber bundle capable of expanding when an insertion cannula is withdrawn to increase an angle over which light is emitted.

FIG. 8A is a schematic diagram of a spiral-like pattern of an embodiment of a side-emitting optical fiber bundle carried by a substrate.

FIG. 8B is a schematic illustration of an embodiment of a side-emitting optical fiber bundle having an S-like pattern and carried by a substrate.

FIG. 8C is a schematic illustration of an end of an embodiment of a side-emitting optical fiber bundle in which the individual optical fibers are carried by a substrate in a distributed manner.

FIG. 9 is a flow diagram of an embodiment of a representative intraventricular implantation process.

FIG. 10A is a flow diagram of an embodiment of a representative optical illumination and electrical stimulation process.

FIG. 10B is a flow diagram of an embodiment of a representative process for specifying a set of reference upper electrical stimulation limit parameters and expanded upper electrical stimulation limit ranges corresponding to one or more electrical stimulation devices.

FIG. 11A is a flow diagram of an embodiment of a representative sensing based neuroillumination process.

FIG. 11B is a flow diagram of an embodiment of a representative near infrared spectroscopy (NIRS) related optical illumination process.

FIG. 12A is a flow diagram of an embodiment of a representative illumination shunt implantation and operation process.

FIG. 12B is a flow diagram of an embodiment of a representative illumination shunt process.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

A wide variety of mental and physical processes are controlled or influenced by neural activity in particular regions of the brain. For example, the neural functions in some areas of the brain (i.e., the sensory or motor cortices) are organized according to physical or cognitive functions. In general, particular areas of the brain appear to have distinct functions in most individuals. Many problems or abnormalities with body functions can be caused by dysfunction, damage, disease and/or disorders in the brain. Effectively treating such abnormalities can be very difficult. Epidemiological profiles indicate that the treatment and/or rehabilitation of neurologic dysfunction is extremely challenging due to patient population heterogeneity, for example, due to factors such as age, gender, ethnicity, cause, physiologic location, severity, and time since onset. For most patients exhibiting neurologic damage arising from, for example, a stroke, conventional treatments are not sufficient, and little can be done to significantly improve the function of an affected body part or cognitive function beyond the limited recovery that generally occurs naturally without intervention.

The present disclosure describes various embodiments of systems, apparatuses, devices, and methods capable of irradiating or illuminating one or more neural populations with light. In general, the application of optical radiation or illumination to neural tissue is referred to herein as neuroillumination. Depending upon embodiment details, various embodiments of systems, apparatuses, devices, and methods in accordance with the present disclosure can irradiate or apply optical signals to one or more portions of the central nervous system (e.g., deep, near-surface, and/or surface brain regions, and/or portions of the spinal cord). In general, the illumination falls within particular portions of the visible and/or near infrared region of the electromagnetic spectrum. Certain embodiments can additionally involve the application of electrical signals and/or chemical substances to one or more neural populations, as further described below.

The beneficial effects of irradiation with certain wavelengths of light at appropriate intensities known to be therapeutic with respect to particular types of neurologic function, dysfunction, diseases, disorders, and injuries include, but are not limited to, increased activity and synthesis of cytochrome oxidase, resulting in increased energy metabolism (e.g., increased mitochondrial metabolism), down-regulation of apoptotic proteins and up-regulation of anti-apoptotic proteins, and decreased generation of reactive oxygen species. Unlike neuromodulation devices that are solely directed toward the application of electrical signals, the neuroillumination systems disclosed herein are intended to affect intraneuronal processes (e.g., intracellular electrochemical processes associated with cellular vitality, intracellular signaling, mitochondrial metabolism, molecular synthesis, and/or electron transport), which can influence a neurochemical environment and/or ultimately affect the inherent progression of a neurological disorder.

According to aspects illustrated herein, light irradiation of neural tissue with the neuroillumination systems disclosed herein can provide or give rise to one or more neuroprotective effects within the irradiated tissue, where such neuroprotective effects can delay or prevent the oxidative damage, oxidative stress, or neurodegeneration of the irradiated tissue. Neural tissue illumination in accordance with particular embodiments disclosed herein can possibly, at least partially, normalize, correct, remedy, or reverse certain effects associated with oxidative stress, neurodegeneration, and/or other adverse neuronal condition, such as decreased mitochondrial metabolism.

In general, light irradiation applied at appropriate wavelengths and intensities, as provided by the neuroillumination systems, methods, processes, and/or techniques disclosed herein, is not intended to trigger the generation of action potentials by neurons as a result of the light irradiation alone, that is, in the absence of extracellular neural input associated with neuroelectric activity involved in neuron-to-neuron communication, or extracellular neural input of an electrical or electromagnetic origin. In other words, illumination applied to a neural target using various embodiments of neuroillumination devices and/or processes detailed below is not in or by itself intended to make neurons within the neural target “fire” action potentials. In general, extracellular neural input associated with neuron-to-neuron communication arises from intrinsic neuron-to-neuron signaling processes (e.g., the propagation of action potentials along or between portions of an anatomical neural network). Extracellular neural input having an electrical or electromagnetic origin can be provided by one or more types of electrical and/or magnetic stimulation devices that apply extrinsic signals extrinsic signals (e.g., electrical or magnetic stimulation signals applied using an implanted electrode or external magnetic coil) to a patient.

Light irradiation applied at appropriate wavelengths and intensities in accordance with multiple embodiments described herein can result in biological effects that persist after the interruption or cessation of light irradiation. Such persistent effects can last, for example, for seconds, minutes, hours, or possibly one or more days.

According to aspects illustrated herein, light irradiation is suitably accomplished by a neuroillumination system of the present disclosure, also referred to herein as a brain neuromodulation system (BNS). In an embodiment, a brain neuromodulation system of the present disclosure includes an implantable device that can be controlled remotely, and which can apply, deliver, or direct radiation, illumination, or optical signals to one or more target neural populations. In an embodiment, the BNS of the present disclosure can include one or more hermetically sealed biocompatible housings that carry or encase device or elements configured to generate, distribute, and apply optical and/or electrical signals to facilitate neural target illumination operations, as further detailed below. In general, a light emitting device can be categorized as an active device or a passive device. A BNS can include one or more active optical devices or elements that generate optical signals in response to other signals (e.g., a light emitting diode (LED), which generates light in response to an applied electrical signal); and/or one or more passive optical devices that transfer, directionally propagate, focus, or wavelength filter optical signals. As further detailed below, illumination can be applied to a neural target by an active light emitting device or a passive light emitting device.

In an embodiment, the BNS can additionally include devices or elements configured to apply electrical signals and/or chemical substances to particular central nervous system and/or other anatomical locations. In an embodiment, the BNS can communicate with and/or include devices or elements configured to detect, sense, or monitor physiologic or physiologic correlate signals, such as neuroelectric signals (e.g., EEG or ECoG signals); optical signals corresponding to the physiologic state of a neural and/or vascular target (e.g., near infrared signals associated with tissue oxygenation level and/or a cytochrome oxidase redox state); and/or particular ionic species, chemical compounds, compositions, or molecular structures (e.g., cytokines).

The brain neuromodulation systems disclosed herein can also include an external programmer/diagnostic system, as well as a patient control unit. One or more implanted neuromodulation devices can be suitably configured for wireless communication with the external programmer/ diagnostic system and/or the patient control unit as appropriate.

Unless otherwise noted, technical terms are used according to conventional usage; as used herein, however, the following definitions may be useful in aiding the skilled practitioner in understanding the disclosure:

As used herein, a “brain neuromodulation system (BNS)” refers to a system that provides neuromodulation of the brain. The BNS may be realized in, for example, a device such as an implantable neurostimulator that provides for light and, optionally, electrical or drug delivery and an external control system.

As used herein, “brain disorder” refers to at least one characteristic or symptom of a neurological, psychiatric, mood, movement, epilepsy, behavioral, addiction, attention, consciousness (e.g., coma), psychological, or other central nervous system disorder. A brain disorder can also be a thought processes disorder, memory disorder, “mental disorder,” neurodegenerative disorder, age-related disorder, cognitive disorder, or other disorder having a neural origin or neural component. Brain disorders can also include pain disorders, migraine, headache, stroke, and other types of traumatic brain injury. Psychiatric disorders can include, for example, forms of psychosis, schizophrenia, depression (e.g., major depressive disorder (MDD) or bipolar disorder), anxiety disorders, post-traumatic stress disorder (PTSD), obsessive-compulsive disorder, or an eating disorder. Any given brain disorder can give rise to abnormalities or irregularities in neural function, generally referred to herein as “neurologic dysfunction.”

Rather than being defined or diagnosed solely based upon particular clinical characteristics such as behavioral or Diagnostic and Statistical Manual of Mental Disorders (DSM) criteria, a brain disorder can additionally or alternatively be defined or diagnosed evidenced through an analysis of neurostructural and/or neurofunctional information. Such information can be acquired by a neuroimaging system (e.g., a Magnetic Resonance Imaging (MRI), functional MRI (fMRI), Magnetic Resonance Spectroscopy (MRS), Positron Emission Tomography (PET), or Magnetoencephaolograpy (MEG) system), an EEG-based (e.g., Quantitative EEG) system, an optical tomography (OT) system, or another type of system. Abnormal neural structures and/or neural activity can be associated with certain areas, or networks of the brain.

As used herein, the terms “treatment regimen,” “treatment program,” “therapy regimen,” and “therapy program” refers to a protocol and associated procedures used to provide a treatment that includes one or more periods during which illumination is applied to a set of neural targets. A treatment or therapy regimen or program can include neural tissue illumination parameters that define particular locations for illumination operations, and parameter values for an illumination/modulation operation or procedure. A treatment regimen can further include a set of patient monitoring operations or procedures, and an evaluation operation or procedure. Depending upon embodiment details, a therapy regimen can define treatment criteria, program instructions, reference or initial parameter values, linking rules, and any other information that is used to provide specific treatment associated with the application of light to a neural target. A treatment regimen can be designed to directly affect or act upon particular neural targets; or indirectly affect certain neural areas or populations as a result of the application of light and possibly electrical signals or chemical substances to particular neural targets (e.g., one or more neural targets within a neural network).

As used herein, the term “target” refers to a particular neural area, region, location, structure, population, or projection (e.g., within the brain) to be irradiated with or exposed to light in association with the treatment of a particular type of neurologic function or neurologic condition, dysfunction, disease, disorder, or injury. Depending on a particular condition under consideration, the irradiated light can be directed at cortical and/or subcortical structures. Cortical structures can be irradiated epidurally or subdurally.

It is fully envisioned that particular embodiments can include implanted devices that combine electrical stimulation and/or chemical substance delivery with light irradiation. In these devices, the target(s) for light irradiation may be the same as or different from than the target(s) for electrical stimulation or chemical substance delivery. It is also fully envisioned that some embodiments are implemented as a closed loop system in which at least one sensing device is used to detect or measure a parameter expected to be relevant to treatment regimen efficacy and/or efficiency. For instance, a closed loop embodiment can include a sensor configured to monitor, measure, or estimate the intensity of light reaching a target (e.g., based upon reflected or backscattered light intensity), and the intensity of emitted light is adjusted to maintain a desired light intensity at or in the target. It is further fully envisioned that certain embodiments are configured as a closed loop system in which one or more parameters (e.g., intensity, duration, and/or duty cycle or timing pattern) corresponding to a specific treatment regimen can vary in response to the measurement and interpretation of physiologic or physiologic correlate information (e.g., the presence, absence, or level of a substance or signal) that is sensed in the brain or elsewhere.

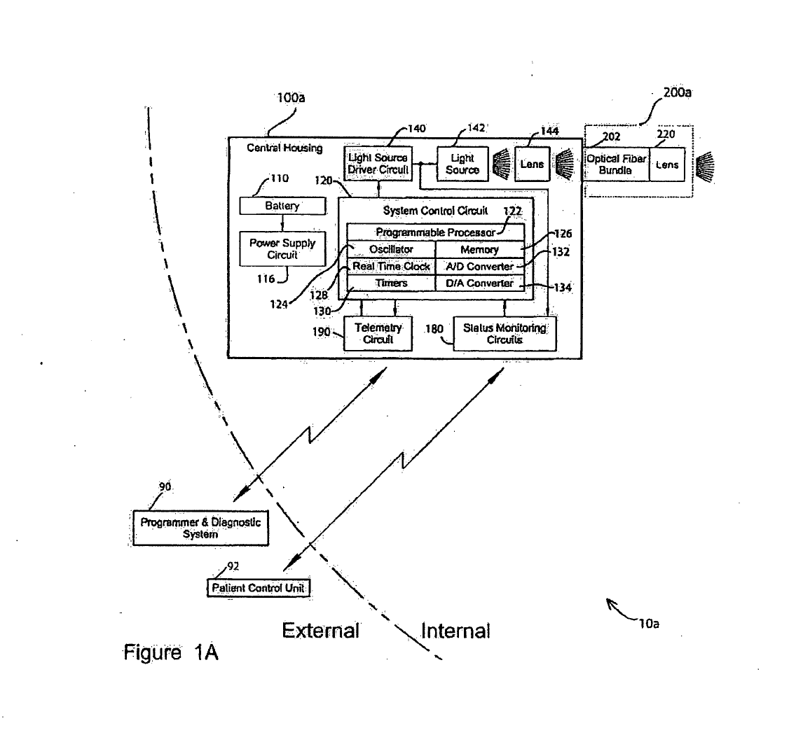

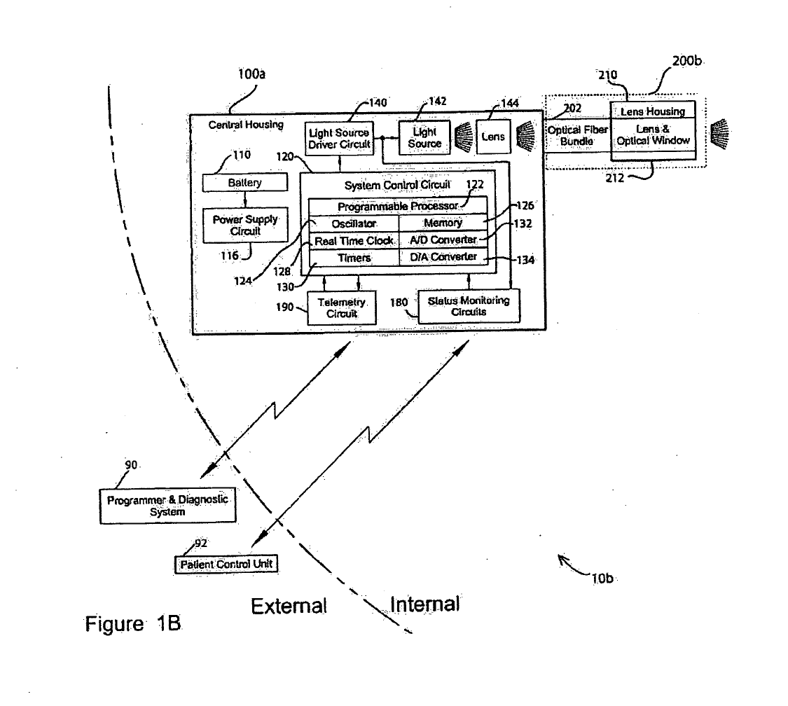

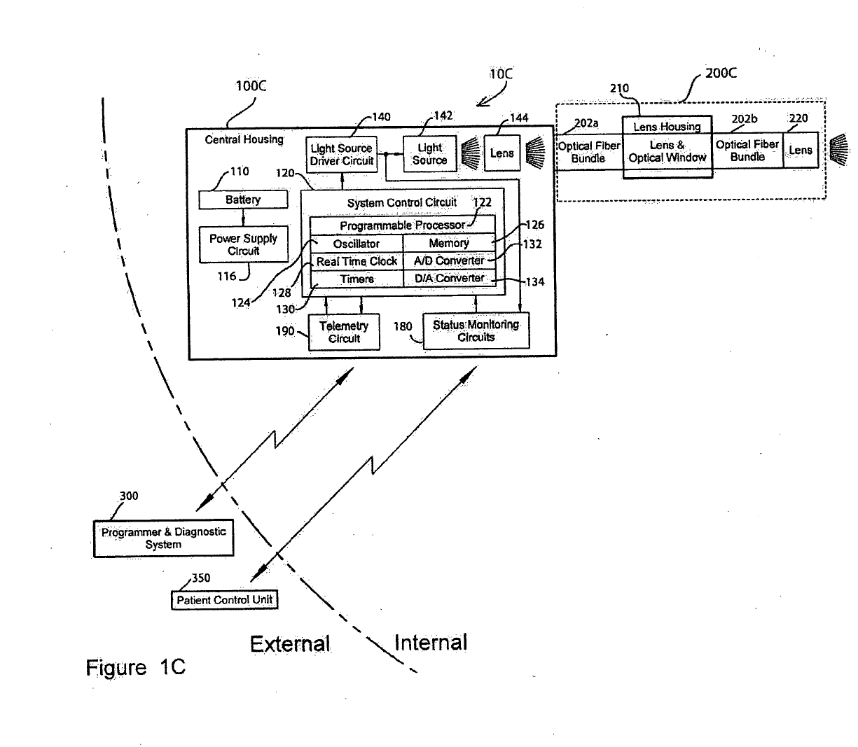

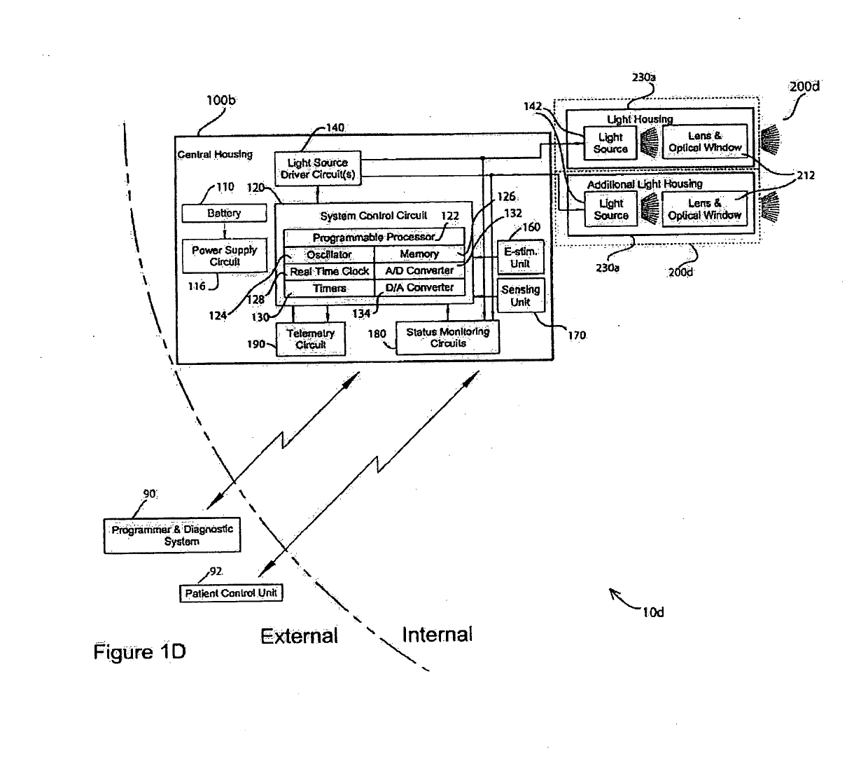

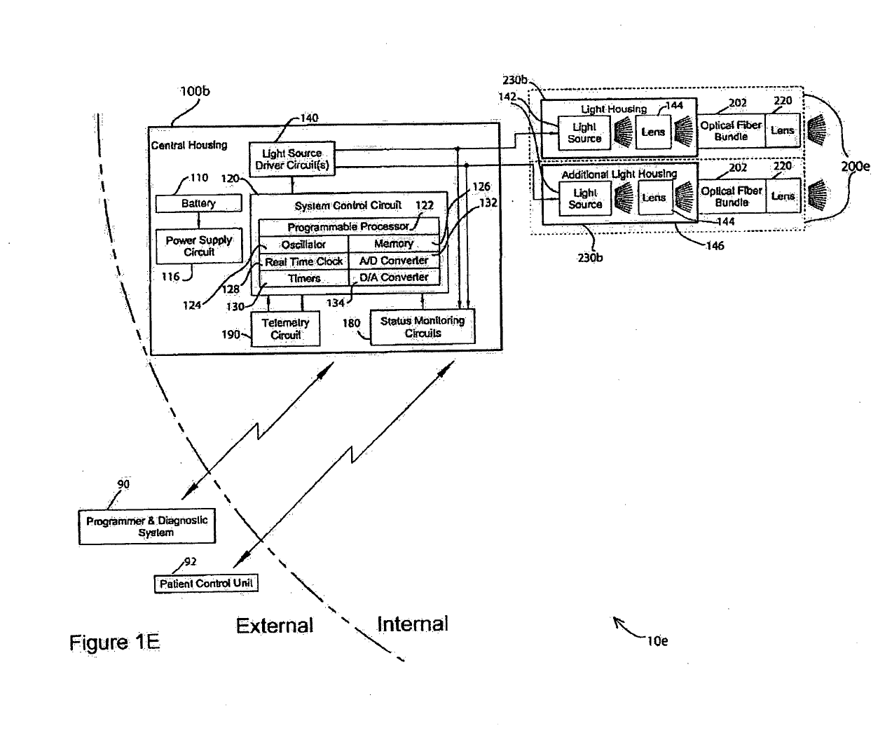

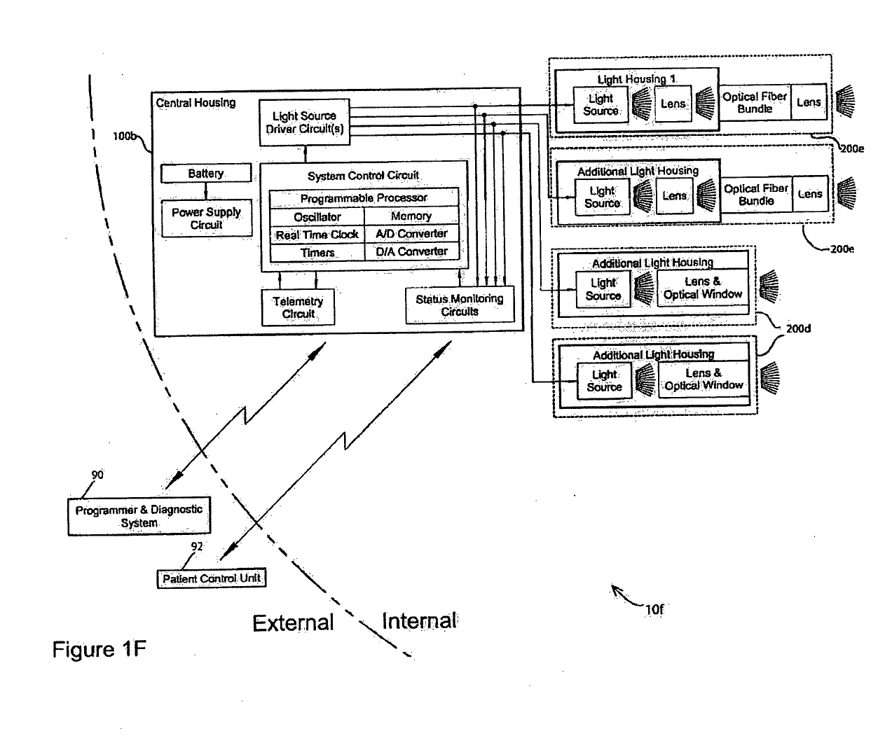

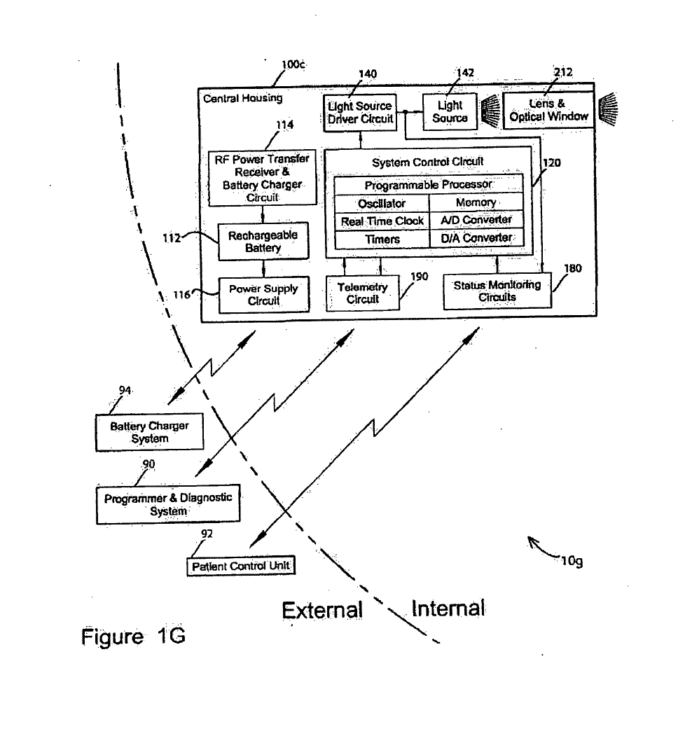

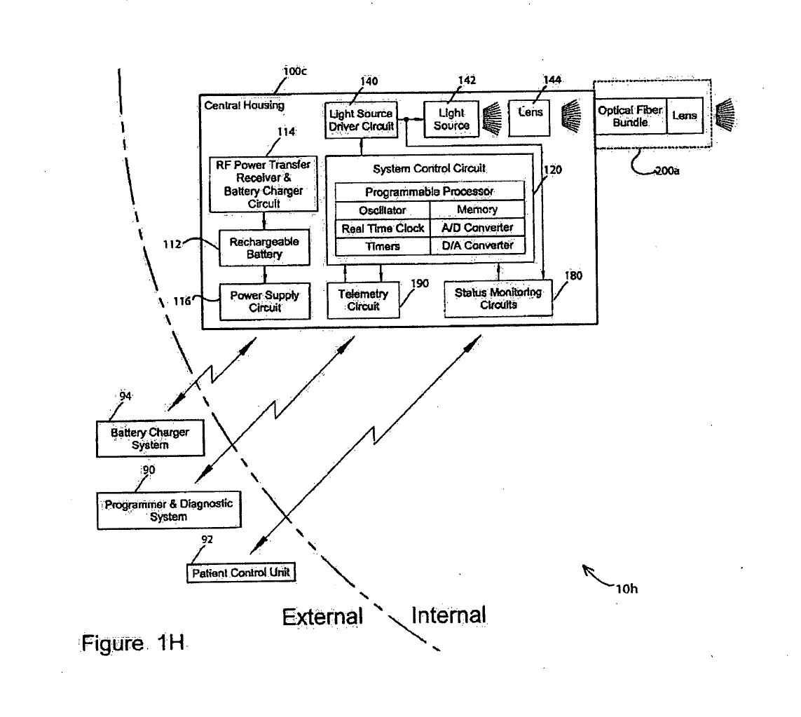

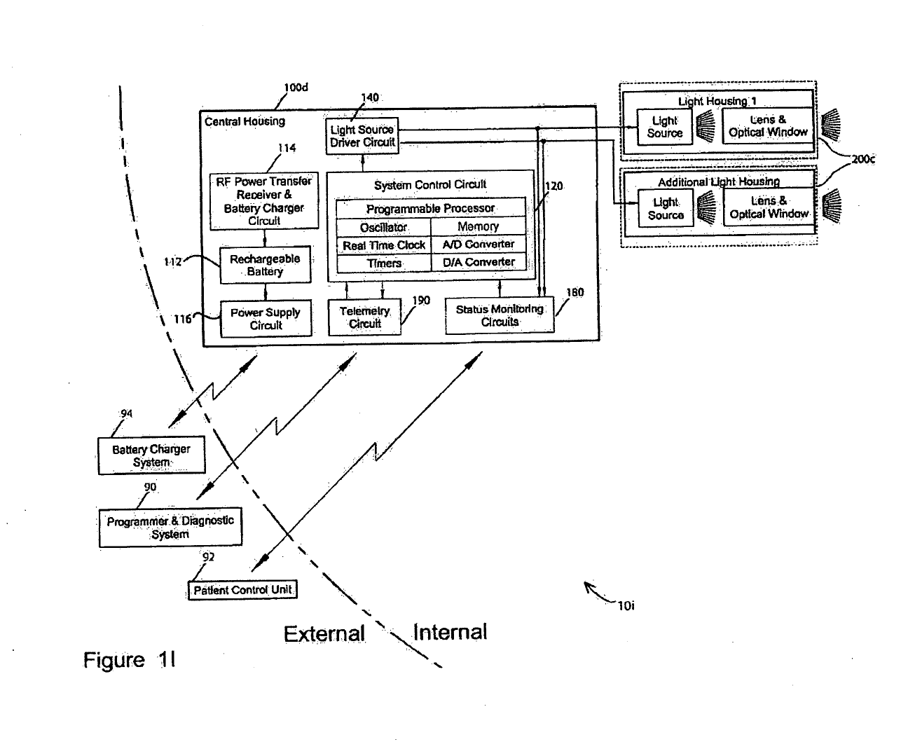

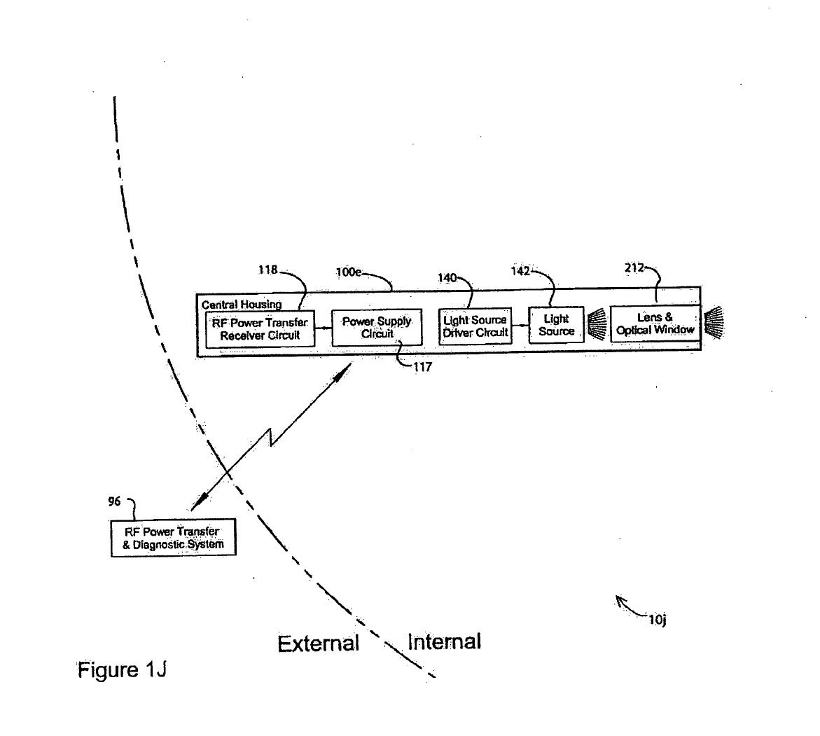

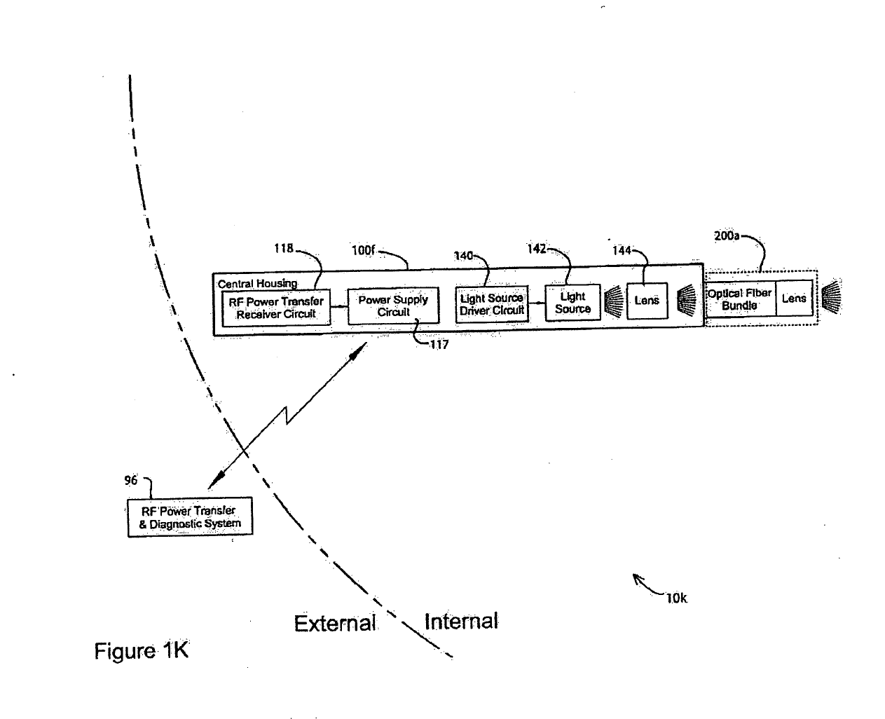

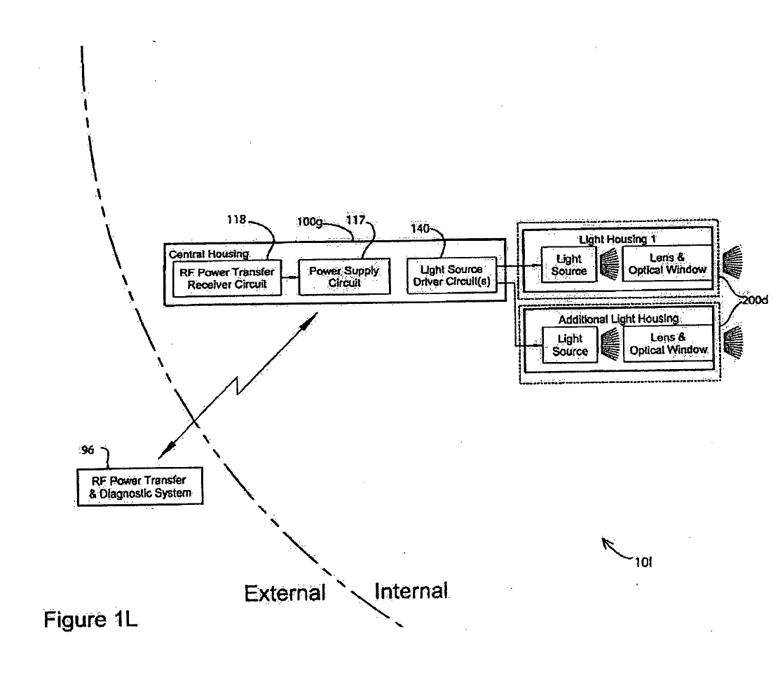

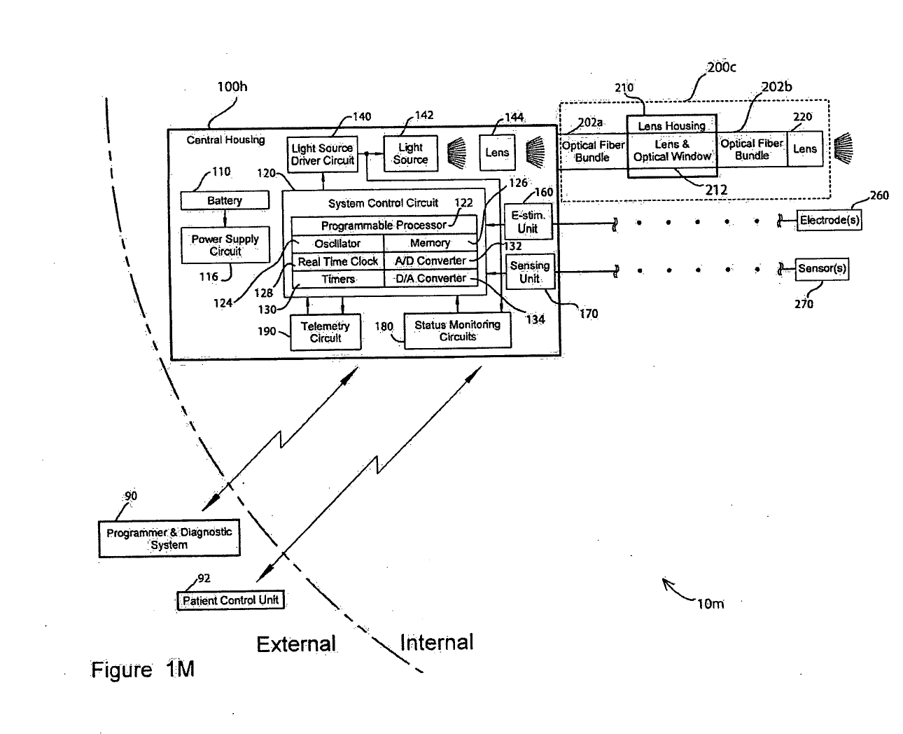

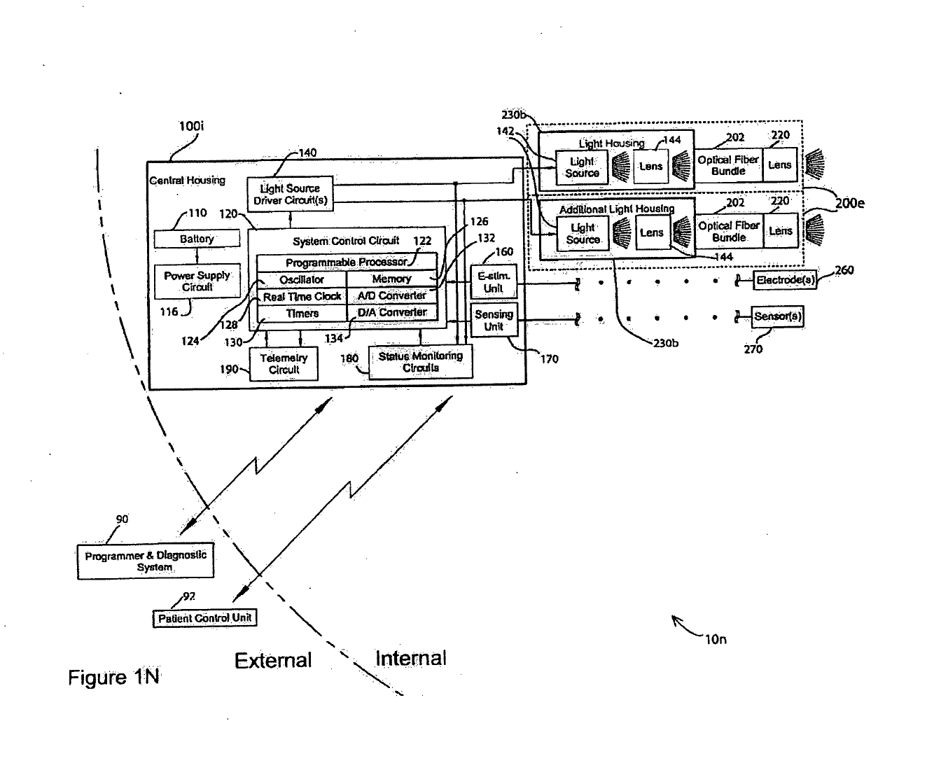

FIGS. 1A-1N are schematic illustrations of various embodiments of brain neuromodulation systems (BNSs) according to the present disclosure. In general, the brain neuromodulation systems include a primary or central implantable housing device and an illumination delivery unit (IDU) coupled to the housing device. The housing device is a biocompatible hermetically sealed housing, which can be Titanium-based. Taken together, the housing device and the IDU form an implantable or implanted device relative to an external/internal boundary defined by a patient's body. The BNS can also include an external programmer and diagnostic system, and optionally, an external patient control unit. In some embodiments, the BNS can further include a power transfer unit configured for wireless power transfer during battery recharging or neural tissue illumination operations.

The primary or central housing includes electrical and possibly optical devices or elements that selectively generate and control or modulate electrical and/or optical signals in accordance with stored program instructions. The IDU receives electrical and/or optical signals output by particular elements within the central housing and can transform or operate upon such signals. Herein, a first light emitting device or element and a second light emitting device or element are defined to be optically coupled or in optical communication when the first light emitting device outputs light that travels or propagates to or through, or is received by, the second light emitting device in accordance with a predetermined or intentional BNS design objective (for example, in the transfer of a beam of light from a light source through a fiber to a lens, the light source, the fiber and the lens are optically coupled or in optical communication). The IDU routes, directs, applies, or delivers optical signals to particular neural targets as described in detail below, such that optical signals are preferentially directed toward, into, and/or through predetermined neural targets associated with a particular patient symptom or type of neurologic function, dysfunction, disease, degeneration, or damage under consideration.

Brain Neuromodulation Systems having a Light Source(s) Internal to a Central Housing

FIG. 1A shows an embodiment of a Brain Neuromodulation System (BNS) 10a of the present disclosure. The BNS 10a includes an implanted primary or central housing device 100a, an IDU 200a coupled to the central housing 100a, an external programmer and diagnostic system 90, and a patient control unit 92.

The central housing 100a carries or includes a power source 110 (e.g., a battery and/or a capacitor), a power supply circuit 116, and a system control circuit 120. The central housing 100a also includes a light source driver circuit 140 operatively coupled to a light source 142 having an optional lens 144. The central housing 100a additionally includes at least one status monitoring circuit 180 and a telemetry circuit 190. The programmer and diagnostic system 90 and the patient control unit 92 are each configured for wireless telemetric signal transfer with the telemetry circuit 190.

The IDU 200a includes an optical fiber bundle 202 having an optional lens 220. The central housing 100a includes a suitable connector or connector assembly (not shown) for coupling, securing, or connecting the optical fiber bundle 202 to the central housing 100a as appropriate, such that light emitted from the light source 142 is directed into the IDU's 200a optical fiber bundle 202. Light emitted by the light source 142 can be directed or focused into the optical fiber bundle 202 by the optional lens 144, which can include one or more lens elements.

The system control circuit 120 manages or controls the generation and application of illumination to neural targets. In various embodiments, the system control unit 120 includes a programmable processor 122, an oscillator 124, and a memory 126. The system control unit 120 can also include one or more of a real time clock 128, a set of timers 130, an analog to digital (A/D) converter 132, and a digital to analog (D/A) converter 134. A bidirectional bus can carry data, address, and control signals between components of the system control circuit 120.

In general, the system control circuit 120 includes discrete and/or integrated electronics configured to perform system control operations according to aspects illustrated herein. In a representative embodiment, the system control circuit 120 includes a programmable processor 122, which can be a commercially available, commercially manufacturable (e.g., as an Application Specific Integrated Circuit (ASIC)), or commercially customizable (e.g., based upon Field Programmable Gate Array (FPGA) technology) programmable microcontroller or microprocessor that exhibits a low power consumption level suitable for a particular application to which the BNS 10a of the present disclosure is directed.

The programmable processor 122 executes a set of program instructions that are stored in the memory 126 to accomplish tasks or operations such as, but not limited to, operating the light source 142 according to the specifications or requirements of a given therapy regimen, communicating with external devices, monitoring the condition of elements such as the light source 142 and the power source 110, storing parameters or program instructions in the memory 126, etc. . . . by exchanging data and control signals with other system control unit 120 or implantable device elements such as the light source driver circuit 140, the status monitoring unit 180, and the telemetry unit 190.

The timers 130, such as those typically incorporated in a commercially available microcontroller, can be configured to form a programmable pulse generator and/or a programmable frequency generator. Output signals from the timers 130 and/or the D/A converter 134 can be used to provide control signals to the light source driver circuit 140. For example, an output signal from the timers 130 can be used as an ON/OFF control signal to the light source driver circuit 140, and an output signal from the D/A converter 134 can be used as an intensity control signal for the light source driver circuit 140.

The memory 126 is suitably non-volatile memory, such as ROM or EEPROM, but can also include volatile memory, such as RAM. The non-volatile memory can include ROM such that the device is manufactured with one or more predetermined sets of stored program instructions. Suitably, at least a portion of the memory 126 is an alterable form of memory, such as EEPROM or RAM, so that parameters, such as ON/OFF timing parameters for a specific therapy regimen, can be altered. Suitably, at least a portion of the memory 126 is non-volatile so that a default program is always stored in memory.

The oscillator 124 can determine a rate at which a program contained within a program storage portion of the memory 126 is executed. Additionally, the system control unit 120 can include a real time clock 128 so that particular tasks, such as the timing of a given therapy regimen, can be scheduled or approximately scheduled at specified times of day.

In response to control signals received from the system control unit 120, the light source driver circuit 140 provides an appropriate current, or voltage, level to activate or energize the light source 142. Suitably, the light source 142 can be energized to emit light continuously or periodically in a variety of manners, in accordance with an appropriate illumination protocol for a particular therapy regimen.

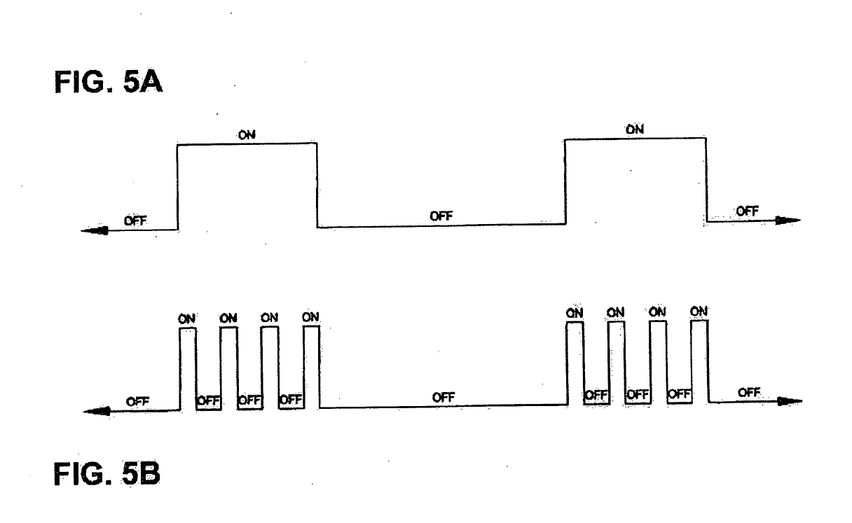

FIGS. 5A and 5B are illustrations of representative types of light source activation or emission patterns with respect to time in accordance with particular embodiments disclosed herein. As shown in FIG. 5A, the light source 142 can be energized to cycle between ON states with continuous emission and OFF states with no emission during particular time intervals or periods; or, as shown in FIG. 5B, the light source 142 can cycle between ON and OFF states during particular subintervals of time. In various embodiments, light source activation can occur in accordance with program instructions that specify particular time and/or intensity based functions or patterns for such activation. Suitably, the light source driver circuit 140 can deliver a fixed level of current, or voltage, to the light source 142 during an ON period so that the intensity of the light emitted remains at a desired or intended level, but the light source driver circuit 140 can additionally or alternatively be configured so that a change in a control signal received from the system control unit 120 causes a change in the level of a current or voltage delivered to the light source 142 so that the intensity of the light emitted changes.

In general, a treatment or therapy procedure or program can specify or include a set of activation times or periods during which the light source 142 is energized or in an ON state, and a set of inactivation or quiescent times or periods in which the light source 142 remains inactive. In an embodiment, the light source 142 can be energized in an ON state for an activation period of approximately 2-20 minutes, or more particularly 3-12 minutes, or more specifically 4-10 minutes; and then switched, reset, or transitioned to an OFF state for a period of seconds, minutes, or hours until a subsequent activation period is scheduled to occur. For instance, the light source 142 can be activated to provide neural tissue illumination for approximately 3-10 minutes, once per day, twice per day, or three times per day. Such light source activation can occur across a therapy or treatment period, such as a given number of days (e.g., a minimum of 2 days, or over 10-30 days), weeks, or months, or on a chronic basis, depending upon a patient state or condition under consideration. In a representative embodiment, the light source 142 is activated to provide continuous neural tissue illumination for approximately 5-10 minutes (e.g., 6 minutes), twice per day.

The light source 142 includes one or more light emitting elements. In various embodiments, the light emitting element is a light emitting diode (LED), but it can be any device that generates or emits light with an appropriate wavelength or spectral distribution and intensity, and which is of small size, low power consumption, and low heat generation. The wavelengths of light produced by or emitted from the light source 142 include wavelengths that fall within the visible or near infrared ranges, approximately 400 nm to 1000 nm, and more particularly between approximately 650 nm and 850 nm (e.g., approximately 670 nm) based on previous studies and the transmission spectrum of tissue. In addition to wavelength, the power density of the light energy applied to the target tissue is an important factor in determining the efficacy of treatment. The light source 142 is capable of emitting light at a power sufficient to achieve a power density in the target tissue between at least 0.01 mW/cm² and 100 mW/cm², and more particularly between 0.1 mW/cm² and 20 mW/cm², and still more particularly between approximately 8 mW/cm² and 12 mW/cm² (e.g., approximately 10 mW/cm²). Depending upon embodiment details, the light energy can be emitted continuously or pulsed during treatment. If pulsed, the pulses can be at least about 10 nanoseconds in duration, and occur at a frequency of up to about 100 kHz.

The light source 142 can include combinations of different light emitting or light generating elements to take advantage of differences such as intensity or wavelength between the various elements. For example, the light source 142 can include two LEDs, with one LED emitting light at a wavelength of approximately 670 nm and the other LED emitting light at a wavelength of approximately 820 nm; or the light source 142 can include multiple LEDs emitting the same wavelength of light in order to emit light of a greater intensity than that emitted by a single LED. When the light source 142 includes combinations of different light emitting elements, the timing (ON/OFF) pattern and/or emitted light intensity can differ between the various elements.

When the light source 142 includes one or more LEDs, the light source driver circuit 140 suitably includes a constant current source that can be switched by a control signal received from the system control unit 120 so that the light source 142 emits light at specific times. When the light source 142 includes one or more LEDs and the light source driver circuit 140 includes a constant current source, a change in a signal from the system control unit 120, such as an output signal from the digital-to-analog converter 134, can be used to change the intensity of the light emitted by causing a change in the output level of the constant current source. When the light source 142 includes combinations of light emitting elements and different timing patterns and/or intensities are required for the various elements, then the light source driver circuit 140 can include separate circuits to control the operation of the various light source elements, with each circuit receiving control signals from the system control unit 120.

The power source 110 can be a battery or other type of energy storage device that can fit within a limited available volume suitable for an implantable medical device. In several embodiments, the power source 110 is suitably a primary battery, but can alternatively or additionally be a rechargeable battery. The advantages of using a primary battery are that it does not require an internal recharging circuit and an external recharging system, and it does not require periodic recharging. The disadvantage of using a primary battery is that it cannot be recharged and consequently the device 100a will typically need to be replaced when the energy of the battery has been depleted. As further described below, a rechargeable battery can be recharged using an AC magnetic field generated external to the patient's body and an internal battery charging circuit to convert RF power received by an inductor into a DC voltage used to recharge the battery. Alternatively, as described elsewhere in this document, RF-powered devices may be used.

The power management circuitry 116 regulates and distributes power to the other elements of the implanted device 100a, as required by the particular types of circuits and components in association with implanted device management and tissue illumination operations.

The status monitoring unit 180 can include a circuit to monitor a condition of the light source 142, such as by measuring a current passing through by the light source 142 and presenting a proportional voltage to an input channel of the analog-to-digital converter 132, the output of which can be read and acted upon by the programmable processor 122. Information related to the condition of the light source 142 can be transmitted to the external programmer and diagnostic system 90 by way of the telemetry unit 190.

The status monitoring unit 180 can also include a temperature sensor, such as a thermistor, and an appropriate signal conditioning circuit to measure a temperature corresponding to the light source 142 or a temperature inside the housing 100a, and present a proportional voltage to an input channel of the analog-to-digital converter 132, the output of which is read and acted upon by the programmable processor 122. Information related to the temperature of the light source 142 or the temperature inside the housing 100a can be transmitted to the external programmer and diagnostic system 90 using the telemetry unit 190.

The status monitoring unit 180 can also monitor or determine a condition of the power source 110, for example, by presenting a battery voltage level to an input channel of the analog-to-digital converter 132, the output of which can be read and acted upon by the programmable processor 122. Information related to the condition of the power source 110 can be transmitted to the external programmer and diagnostic system 90 by the telemetry unit 190.

The telemetry unit 190 can cooperatively interact to establish a wireless communication link between the implanted device 100a and devices external to the patient's body, such as the programmer and diagnostic system 90 and/or the patient control unit 92, which also include corresponding telemetry circuits. The telemetry unit 190 suitably transmits and receives modulated RF signals, but other types of wireless communication links can be used. The telemetry unit 190 receives information, such as parameters specific to one or more portions of a treatment regimen, from external devices, and transmits information, such as the condition of the power source 110 or light source 142, to external devices. In various embodiments, a wireless communication link request originates from the programmer and diagnostic system 90, the patient control unit 92, or another external device.

The external programmer and diagnostic system 90 includes a telemetry circuit, generally of the same type as the telemetry unit 190 included in the implanted device 100a, to form a wireless communication link with the implanted device 100a. The programmer and diagnostic system 90 uses the wireless communication link to program and interrogate the implanted device 100a, such as by transmitting information, such as parameters corresponding to one or more portions of a therapy regimen, to the implanted device 100a, and by receiving information, such as the condition of the power source 110 or light source 142, from the implanted device 100a. Typically, the programmer and diagnostic system 90 is used by a clinician.

The external patient control unit 92 includes a telemetry circuit, of the same general type included in the implanted device 100a, to form a wireless communication link with the implanted device 100a. The patient control unit 92 uses this wireless communication link to communicate with or control the implanted device 100a in particular manners, such as by transmitting commands to enable or disable a programmed therapy regimen. The patient control unit 92 can also use the wireless communication link to receive information, such as the condition of the power source 110 or light source 142, from the implanted device 100a. In multiple embodiments, however, the patient control unit 92 is an on-off controller for the emission of light signals.

In the embodiment shown in FIG. 1A, the IDU 200a includes, carries, or incorporates an optical fiber bundle 202 and an optional lens 220. One end of the optical fiber bundle 202 is coupled, connected, or attached to the central housing 100a. The optical fiber bundle 202 includes one or more optical fibers, and is constructed and sealed in a manner suitable for implantation. Implanted optical fiber bundles have been described in U.S. Pat. Nos. 6,238,409, 6,091,015, 7,395,118, and 7,190,993, each of which is incorporated herein by reference in its entirety.

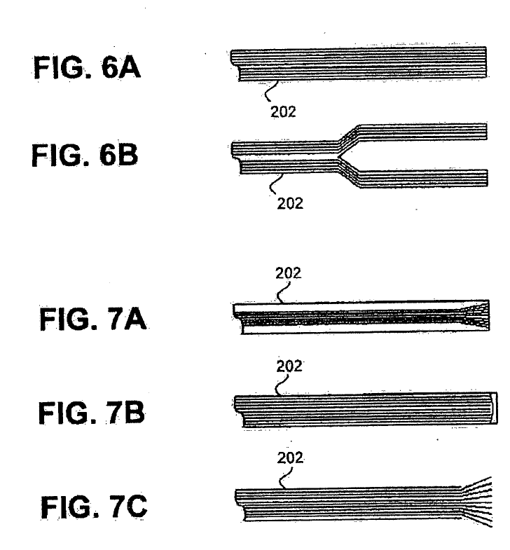

FIG. 6A is an illustration of an embodiment of an optical fiber bundle 202 having a single segment, and FIG. 6B is an illustration of an embodiment of an optical fiber bundle 202 having multiple segments. The optical fiber bundle 202 can have a single segment as illustrated in FIG. 6A, or can be have multiple segments or branches as illustrated in FIG. 6B. When an optical fiber bundle 202 is divided into multiple branches, particular branches can be positioned to irradiate light into different or identical neural targets. For example, distinct branches of the optical fiber bundle 202 can be positioned to irradiate or direct light into different bilateral targets; neuroanatomically corresponding (e.g., homotypic) bilateral targets; different unilateral targets; or an identical unilateral target.

FIGS. 7A through 7C are illustrations of various embodiments of optical fiber bundle tip regions. The optical fiber bundle 202 can emit light from the tip, and/or from one or more locations on a side or surface. Typically, the optical fibers are parallel to each other in the optical fiber bundle 202, but the optical fibers can be flared near the tip to increase an angle over which light is emitted from the tip as illustrated in FIG. 7A. A lens can be coupled, connected, attached, or adhered to the tip of the optical fiber bundle 202 to control an angle over which light is emitted from the tip, as illustrated in FIG. 7B; or the optical fibers can expand near the tip of the optical fiber bundle 202 after an insertion cannula is removed as illustrated in FIG. 7C.

Side-emitting optical fiber bundles 202 can emit light from the entire optical fiber bundle 202 periphery or circumference, or one or more portions the circumference. Additionally, side-emitting optical fiber bundles 202 can emit light over one or more specific sites, sections, locations, or positions along an optical fiber bundle's 202 length. Furthermore, side-emitting optical fiber bundles 202 can be carried by, arranged upon, or at least partially embedded within a substrate (e.g., a flexible or generally flexible Silicone substrate) in accordance with a predetermined layout or pattern.

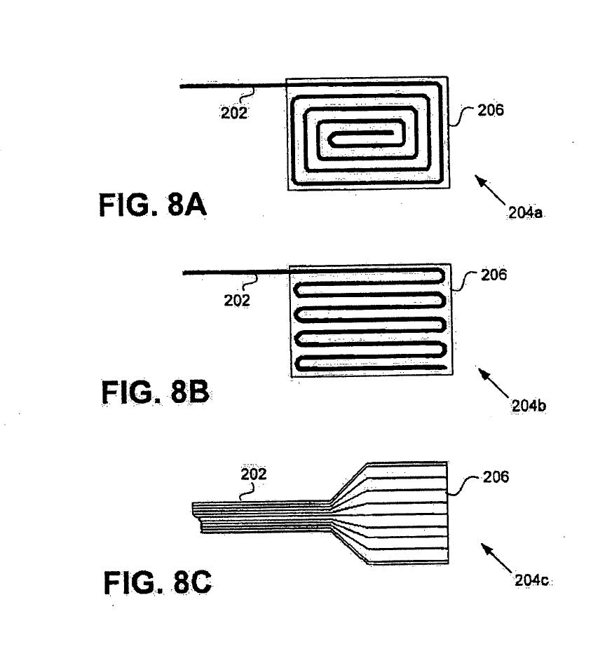

FIGS. 8A, 8B, and 8C are schematic diagrams of a first, a second, and a third illumination paddle 204a, 204b, 204c, respectively, according to various embodiments. Any given illumination paddle 204a, 204b, 204c includes a side-emitting optical fiber bundle 202 arranged or positioned in a given geometric pattern; and a substrate 206 upon and/or within which portions of the side-emitting optical fiber bundle 202 reside. The first illumination paddle 204a includes a side-emitting optical fiber bundle 202 configured in a spiral type pattern, and the second illumination paddle 204b includes a side-emitting optical fiber bundle 202 configured in an S-like pattern. The third illumination paddle 204c includes a side-emitting optical fiber bundle 202 having a fan-like region in or across which subsets of optical fibers are distributed.

The particular type (e.g., single or multi-segment, probe-type or paddle-type), length or size, and light emission or light delivery characteristics (e.g., the number and type(s) of light emission sections, and/or emitted light focality) of the optical fiber bundle(s) 202 can be based upon one or more types of patient conditions under consideration, and particular neural targets corresponding to such patient conditions. For instance, a tip-emitting optical fiber bundle 202 can be used when an optical fiber bundle tip is directed at, but outside of, a target; a tip-emitting and side-emitting optical fiber bundle 202 can be used when the tip is positioned inside a target; a full-circumference side-emitting optical fiber bundle can be used when the optical fiber bundle 202 extends through one or more portions of a target; and a partial-circumference side-emitting optical fiber bundle 202 can be used when a portion of the length of the optical fiber bundle 202 is positioned adjacent or proximate to a target. Any of the first, second, or third illumination paddles 204a, 204b, 204c can be implanted epidurally or subdurally to irradiate light into surface regions of the brain, such as surface accessible cortical targets. Additionally or alternatively, an illumination paddle 204a, 204b, 204c can be used to provide spinal column irradiation (SCI) to irradiate one or more neural targets within the spinal cord (e.g., following spinal column injury).

Referring again to the particular embodiment shown in FIG. 1A, light emitted by the light source 142 travels through the optional lens 144 within the central housing 100a; the optical fiber bundle 202; and the optional lens 220 coupled to a distal end of the optical fiber bundle 202 before emanating into one or more neural targets. In such an embodiment, the distal end of the optical fiber bundle 202, and/or the optional lens 220 that resides at the distal end of the optical fiber bundle 202, control the size and shape of the beam of light that is emitted into particular neural target(s).

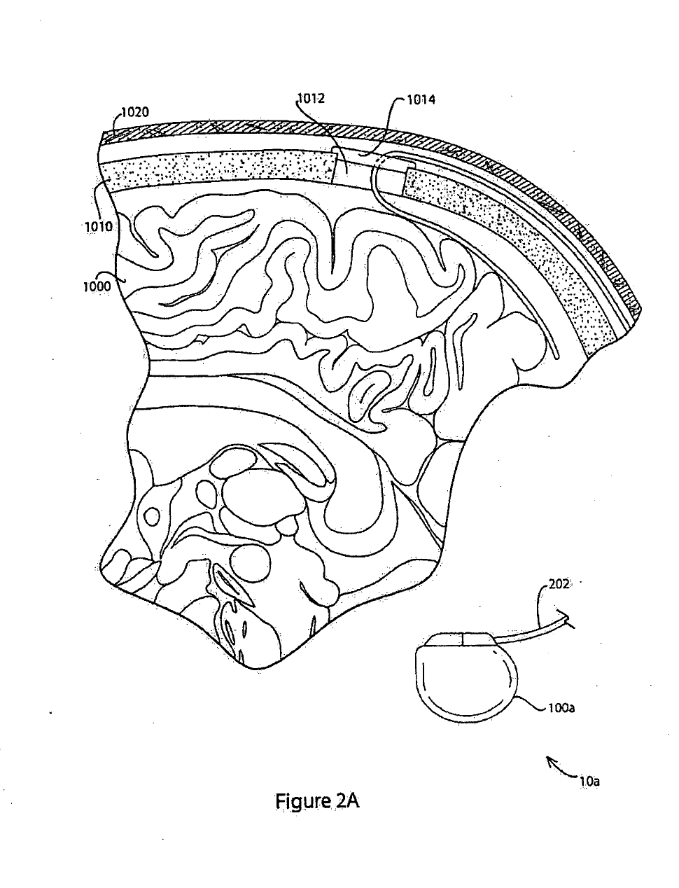

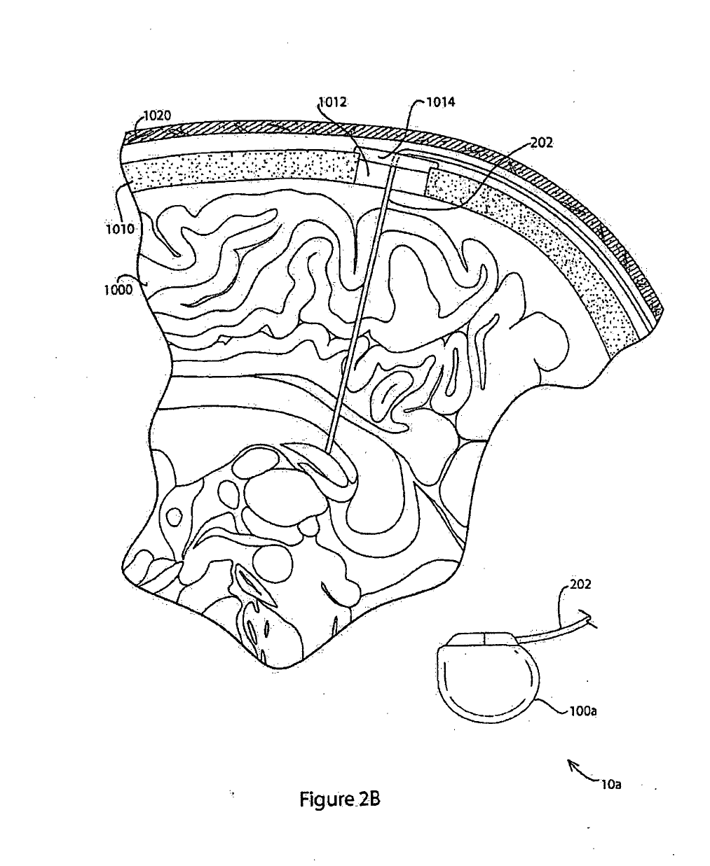

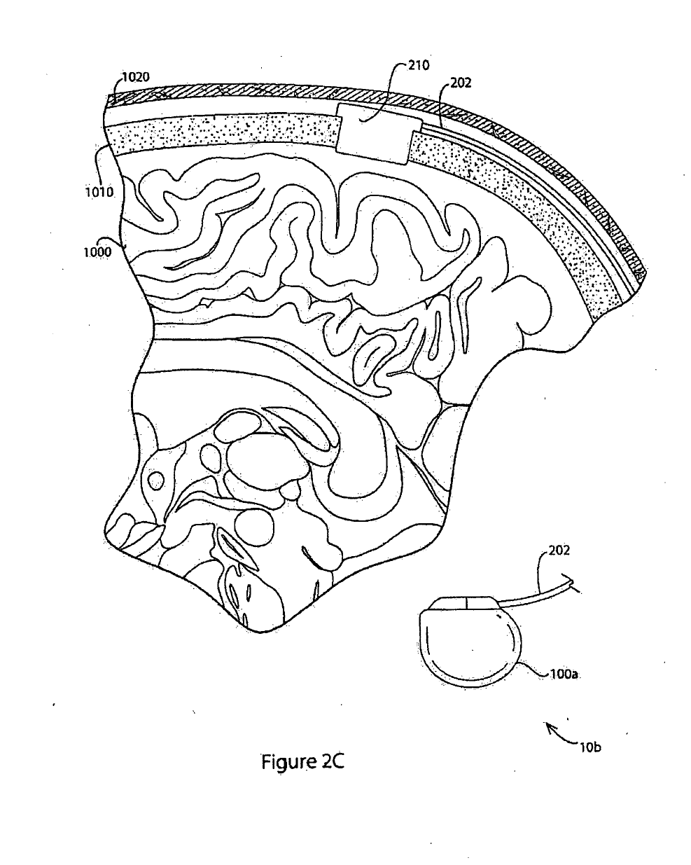

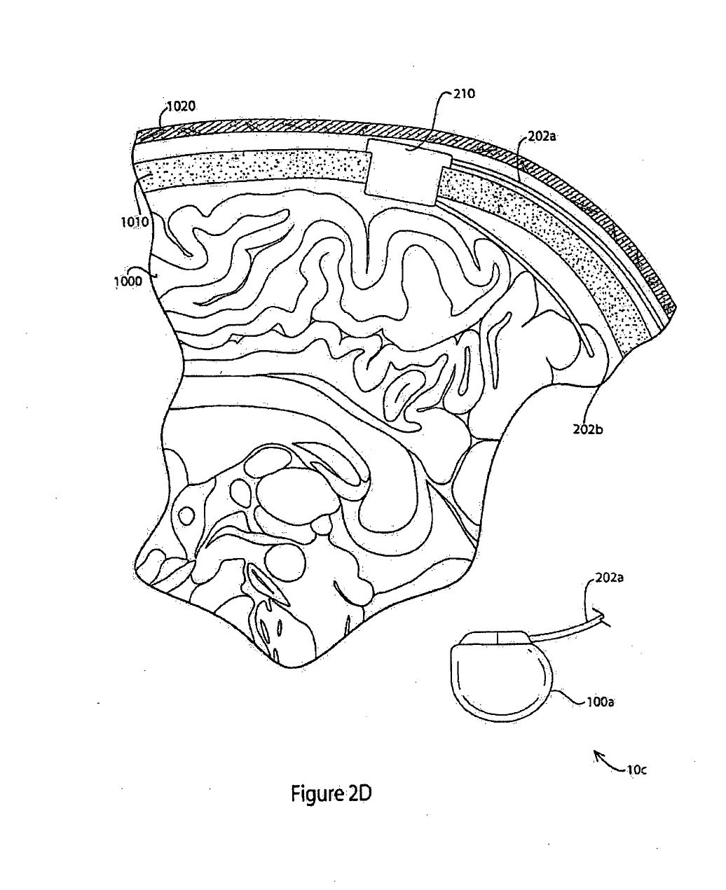

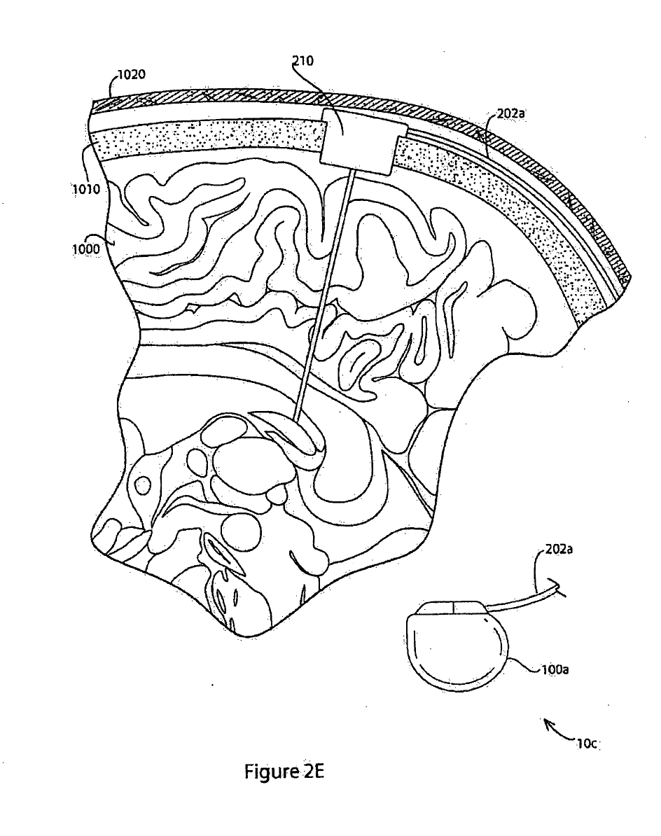

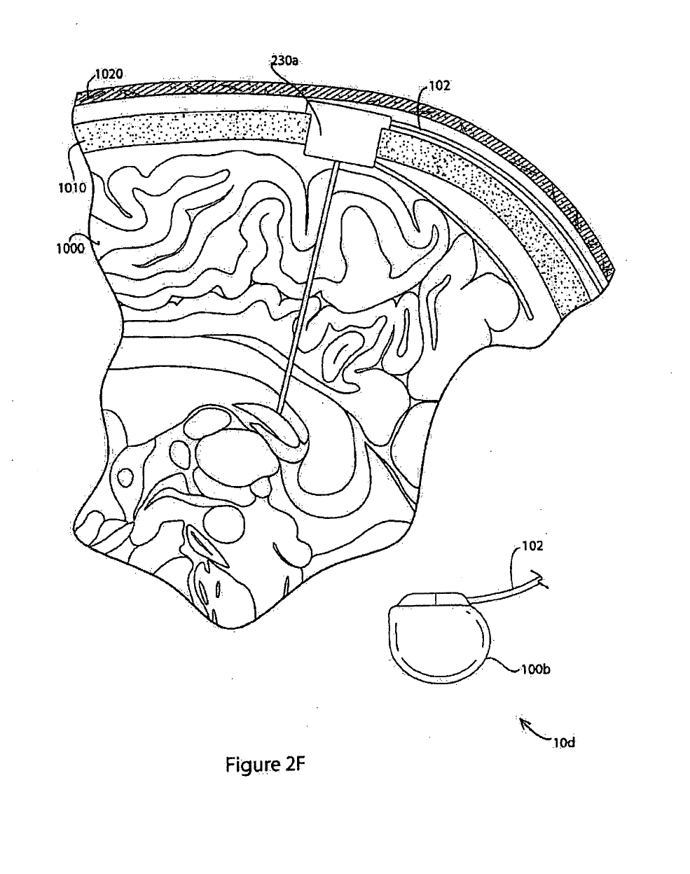

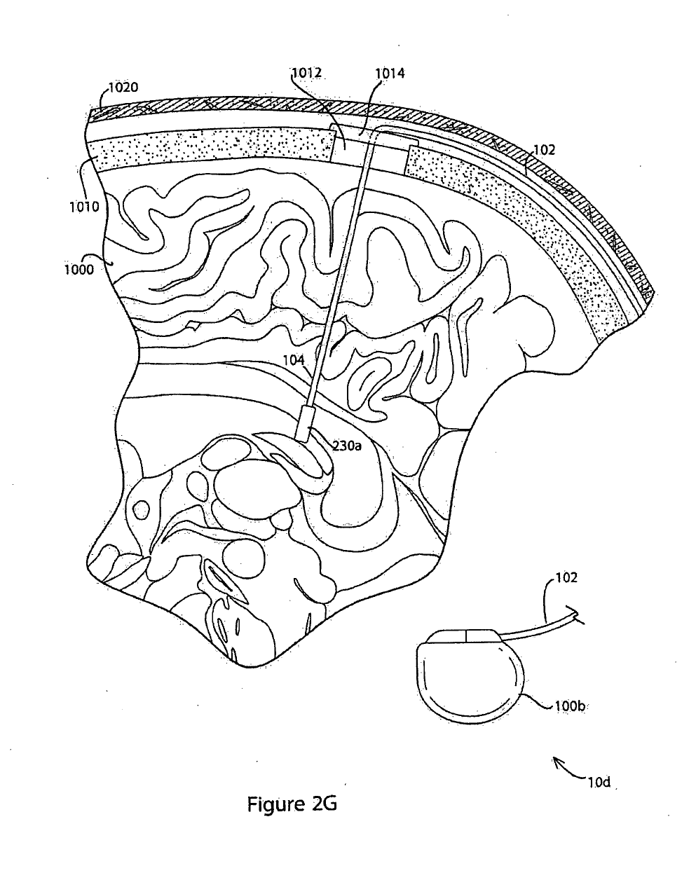

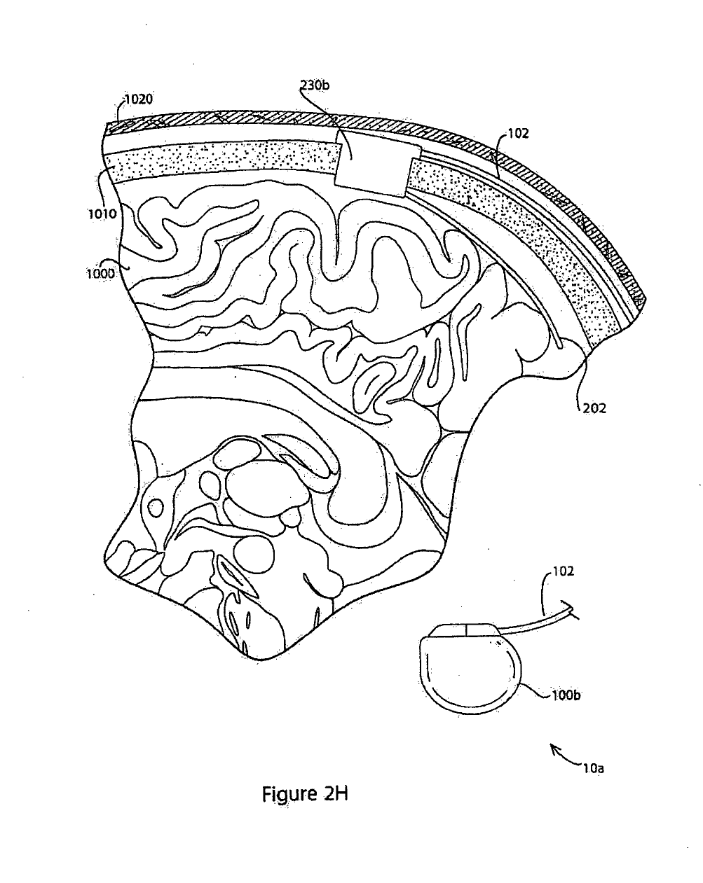

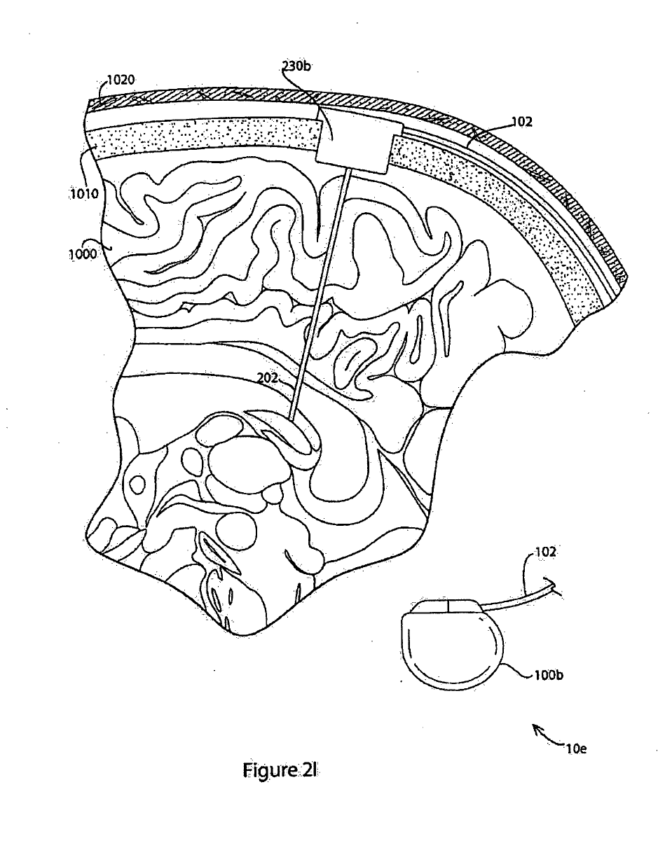

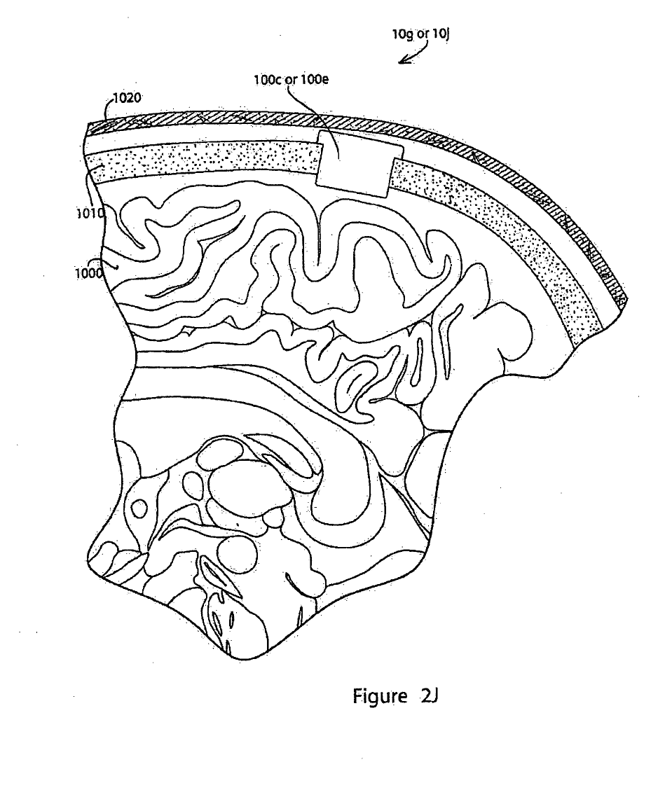

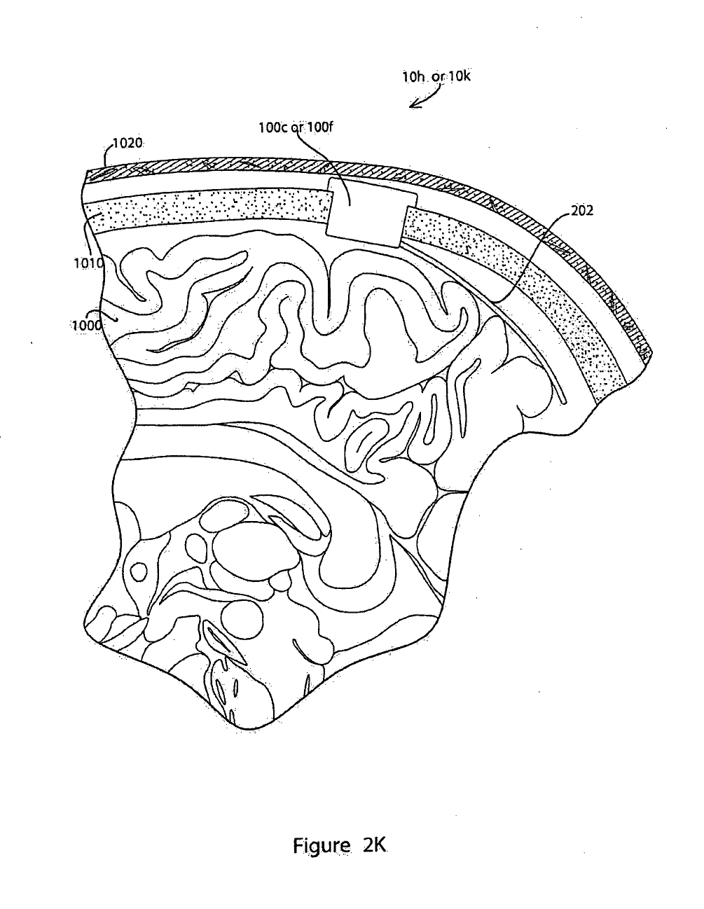

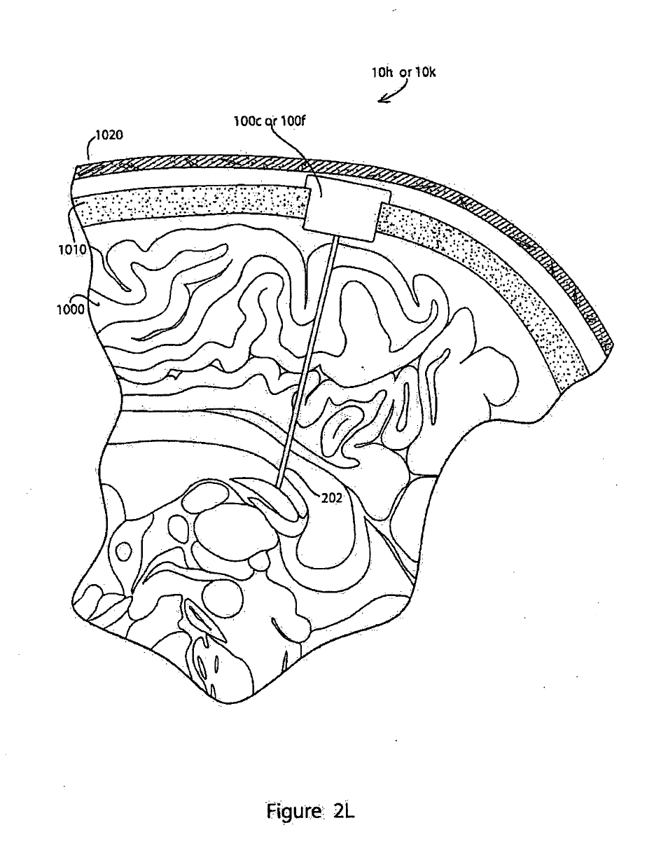

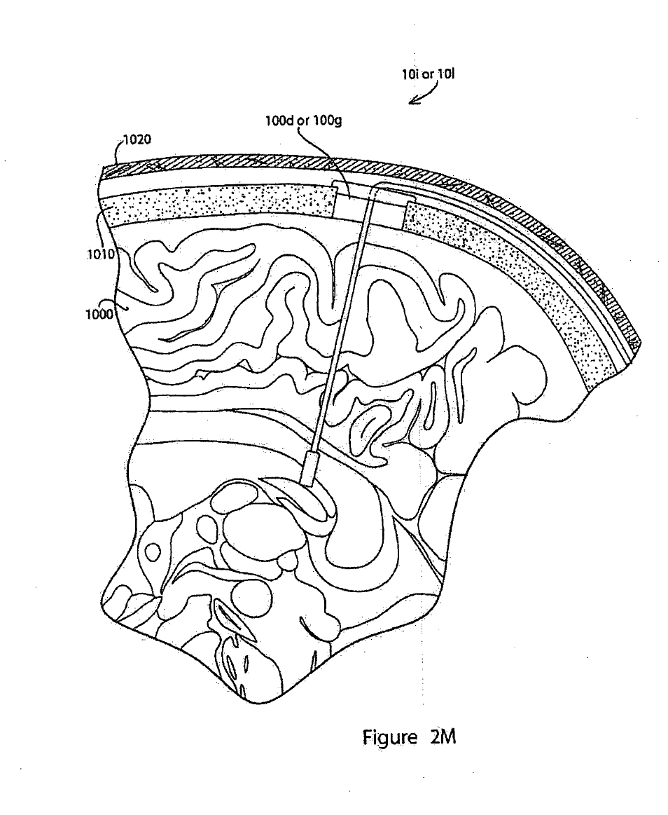

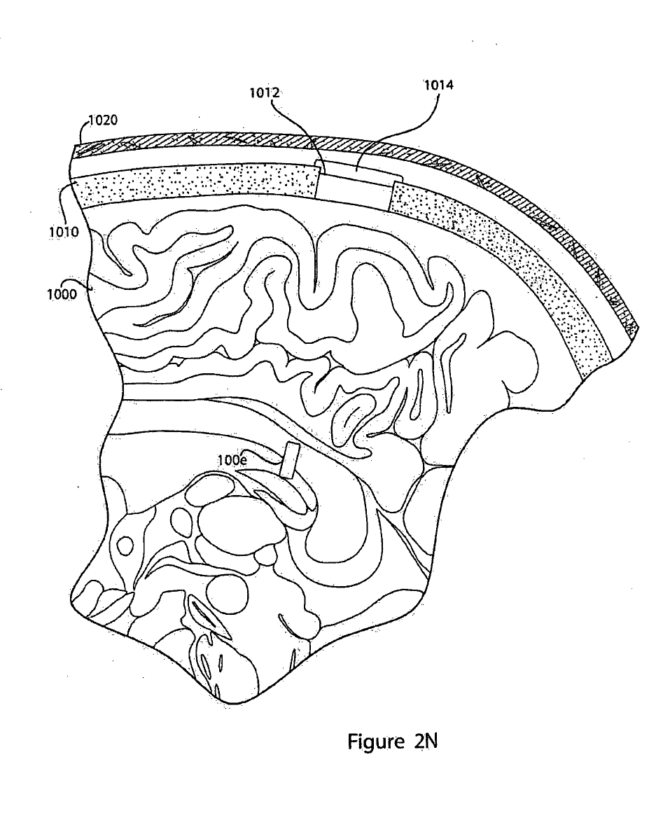

FIGS. 2A and 2B are representative embodiments of the BNS 10a of FIG. 1A, configured for cortical and subcortical (e.g., deep brain) neural target illumination, respectively. Also referring again to FIG. 1A, the central housing 100a is implanted beneath the patient's skin, for example, in a subclavicular, abdominal, or other anatomical region. The optical fiber bundle 202 extends beneath the patient's skin 1020 from the central housing 100a into the brain 1000, passing through an opening or hole 1012 in the skull 1010. A cover 1014 over the hole 1012 restrains the optical fiber bundle 202. The optical fiber bundle 202 is positioned such that the light emitted from the optical fiber bundle 202 is directed to one or more specific neural targets in the brain. An optical fiber bundle 202 configured to apply optical radiation to a subcortical neural target can be considered to be or form a portion of a subcortical or deep brain neuroillumination probe.

FIG. 1B is a simplified block diagram of an embodiment of a BNS 10b of the present disclosure. The BNS 10b includes the implanted central housing 100a, the external programmer and diagnostic system 90, and the optional external patient control unit 92, each of which can be identical or substantially identical to those previously described with reference to FIG. 1A. The BNS 10b further includes an IDU 200b having the optical fiber bundle 202 and a lens housing 210 in which a lens module 212 resides. The optical fiber bundle 202 has a first end that is coupled, connected, or attached to the implanted central housing 100a, and a second end that is coupled, connected or attached to the lens housing 210. The lens module 212 includes a lens and an optical window, where the lens can include one or more lens elements. The optical window can be distinct from or an integral portion of the lens.

Light emitted from the light source 142 passes through any central housing lens 144, and enters the optical fiber bundle 202. Such light further passes through the lens and optical window in the lens module 212, and emanates into the brain. The lens module's 212 lens and optical window control the size and/or shape of the beam of light emitted into the brain.

FIG. 2C is a representative embodiment of the BNS 10b of FIG. 1B, configured for cortical neural target illumination. Also referring again to FIG. 1B, the central housing 100a is implanted beneath the patient's skin (e.g., subclavicularly), and the optical fiber bundle 202 extends beneath the patient's skin 1020 from the central housing 100a to the lens housing 210, which resides or is seated and/or fastened at least partially within the opening in the patient's skull 1010. Light emitted or transmitted from the lens housing's 210 lens and optical window irradiates one or more portions of the brain. An embodiment such as that shown in FIG. 2C is suitable for irradiating cortical targets.

FIG. 1C is a simplified block diagram of an embodiment of a BNS 10c of the present disclosure. The BNS 10c includes the implanted central housing 100a, the external programmer and diagnostic system 90, and the optional external patient control unit 92, each of which can be identical or substantially identical to those previously described with reference to FIG. 1A. The BNS 10c further includes an IDU 200c having a first optical fiber bundle 202a, a lens housing 210, a second optical fiber bundle 202b, and an optional terminal or distal lens 220. The first optical fiber bundle 202a is suitably coupled, connected, or attached between the central housing 100a and an input portion of the lens housing 210; and the second optical fiber bundle 202B is coupled, connected, or attached between an output portion of the lens housing 210 and the terminal lens 220. The lens housing 210 includes a lens module 212, which directs light carried by, traveling through, or emitted by the first optical fiber bundle 202a into the second optical fiber bundle 202b. Any lens 220 at the end of the second optical fiber bundle 202b controls the size and/or shape of the beam of light emitted or directed into a neural target.

FIGS. 2D and 2E are representative embodiments of the BNS 10c of FIG. 1C. The BNS 10c shown in FIG. 2D is configured to provide cortical neuroillumination, while the BNS 10c shown in FIG. 2E is configured to apply subcortical or deep brain neuroillumination. The central housing 100a in FIGS. 2D and 2E is implanted below the patient's skin 1020 at a location that is remote from the opening in the skull 1010 in which the lens housing 210 is seated, fastened, and or affixed.

Relative to FIG. 2E, and other embodiments described herein, an optical fiber bundle that provides or delivers optical signals to a subcortical neural target can be or form a portion of a subcortical or deep brain neuroillumination probe. In some embodiments, an optical fiber bundle has an elongated, cylindrical structure. In certain embodiments, an optical fiber bundle can be carried or surrounded by or encased within another element or material, such as a stainless steel or titanium tube, rod, shaft, or probe. In other embodiments, an optical fiber bundle can have or form elongated cylindrical structure having an opening therein, such that the optical fiber bundle can surround or encase a shaft, probe, or needle-like structure.

Brain Neuromodulation Systems having a Light Source(s) External to a Central Housing

FIG. 1D is a simplified block diagram of an embodiment of a BNS 10d of the present disclosure. The BNS 10d includes a central housing device 100b, at least one IDU 200d, the external programmer and diagnostic system 90, and the optional patient control unit 92. In the embodiment shown in FIG. 1D, one or more light sources 142 reside inside the one or more IDUs 200d. The central housing 100b carries or includes the power source 110, the power supply circuit 116, the system control unit 120, the status monitoring unit 180, and the telemetry unit 190. The central housing 100b additionally carries the light source driver circuit 140, which is coupled to the one or more light sources 142 that are carried by the at least one IDU 200d. The coupling between the central housing 100b and any given IDU 200d can be provided by one or more suitably insulated lead bodies, lead lines, or cables 102 (shown below with reference to FIG. 2F) that enclose, surround, or encapsulate a set of wires that carry or transmit electrical signals output by the light source driver circuit 140.

The central housing 100b, by way of the light source driver circuit 140, provides electrical activation and/or control signals to the IDU 200d to facilitate the generation of optical signals and the delivery of such optical signals to targeted neural tissue. In the embodiment shown, the IDU 200d includes at least one illumination housing 230a that carries the light source 142 and the lens module 212. The light source 142 receives electrical signals output by the light source driver circuit 140, and accordingly generates, emits, or outputs light signals. The light output by the light source 142 travels into the lens module 212, which can include a set of lenses and an optical window (which may be an integral portion of a lens) that focus and/or shape a light beam directed toward illuminating a neural target.

The BNS 10d can include multiple illumination housings 230a, which can have identical or different types of light sources 142. Accordingly, the central housing 100b can include separate or different light source driver circuits 140 to control the operation of the various light sources 142. Similarly, the status monitoring unit 180 can include separate circuits or subcircuits to monitor the condition of various light sources 142.

FIG. 2F illustrates one representative embodiment of the BNS 10d of FIG. 1D, configured for cortical neural target illumination. The central housing 100b is implanted in a manner previously described, and at least one illumination housing 230a is attached, seated, or affixed in an opening in the patient's skull 1010. A lead body or lead line 102 electrically couples the central housing 100b to the light source 142 within the illumination housing 230a. An embodiment such as that shown in FIG. 2F is suitable for optically irradiating cortical targets.

FIG. 2G illustrates another representative embodiment of the BNS 10d of FIG. 1D. Relative to other Figures herein, like reference numbers indicate like or corresponding elements. In the embodiment shown in FIG. 2G, the illumination housing 230a is implanted within the brain (e.g., to provide deep brain illumination). Any given illumination housing 230a is positioned such that emitted light is directed into particular neural targets or structures within the brain. In such an embodiment, conductive wires that facilitate electrical signal communication between the central housing 100b and the illumination housing 230a are carried by or embedded within a lead line 102, and possibly further carried by an elongated or shaft portion of a neuroillumination probe 104. The neuroillumination probe 104 can include or carry the illumination housing 230a, such that the illumination housing 230a forms a portion of the illumination probe 104. The lead line 102 extends between the central housing 100b and a cover 1014 over the opening 1012 in the patient's skull 1010. The elongated member or shaft portion of the neuroillumination probe 104 extends from the cover 1014 into the brain, and terminates at the illumination housing 230a. The lead line 102 and the neuroillumination probe 104 can be electrically coupled or connected in a manner readily understood by one of ordinary skill in the art (e.g., in a manner analogous to a lead extension).

FIG. 1E is a simplified block diagram of an embodiment of a BNS 10e of the present disclosure. Relative to FIG. 1F, like reference numbers indicate like or corresponding elements. The BNS 10e includes the central housing 100b (as described previously for FIG. 1D) that is electrically coupled to one or more IDUs 200e, where at least one IDU 200e includes an illumination housing 230b, the optical fiber bundle 202, and the optional terminal lens 220. The illumination housing 230b can include the light source 142 and the optional lens 144. As with the BNS 10d shown in FIG. 1D, a set of IDUs 200e can include identical or different types of light sources 142, and thus corresponding considerations apply to the light source driver circuit 142 and/or status monitoring circuit 180 in the embodiment shown in FIG. 1E.

Additionally, when the central housing 100b is coupled or connected to more than one IDU 200e, different illumination housings 230b can be coupled or connected to identical or different types of optical fiber bundles 202. For example, one illumination housing 230b can be attached to an end-emitting optical fiber bundle 202, and another illumination housing 230b can be attached to a side-emitting optical fiber bundle 202.

Light generated or emitted by the light source 142 can travel through the optional lens 144 within the illumination housing 230b, after which the light travels through the optical fiber bundle 202 and the optional lens 220 on the terminal or distal end of the optical fiber bundle 202.

FIGS. 2H and 2I respectively illustrate representative cortical illumination and subcortical illumination embodiments of the BNS 10e of FIG. 1E.

In addition to the foregoing, a given BNS 10 can include multiple types of IDUs 200. FIG. 1F is a simplified block diagram of an embodiment of a BNS 10f of the present disclosure. Relative to previous Figures, like reference numbers indicate identical, corresponding, or similar elements. The BNS 10f includes at least one IDU 200d of a type described above with reference to FIG. 1D, and at least one IDU 200e of a type described above with reference to FIG. 1E. Such IDUs 200d, 200e can be structurally and functionally identical or analogous to those in FIGS. 1D and 1E. Representative embodiments of the BNS 10f shown in FIG. 1F can correspond to one or more portions of brain implantation illustrations shown in previous Figures, such as the illustrations shown in FIGS. 2F, 2G, 2H, and/or 2I.

### Rechargeable and RF Powered Brain Neuromodulation Systems

FIGS. 1G, 1H, and 1I are simplified block diagrams in which a BNS of the present disclosure includes a rechargeable or replenishable power source, such as a rechargeable battery and/or a capacitor. Relative to other Figures herein, like or analogous reference numbers indicate like, generally equivalent, corresponding, or analogous elements.

Each of brain neuromodulation systems 10g, 10h, and 10i (of FIGS. 1G, 1H and 1I, respectively) includes a rechargeable power source 112 and a power receiver/battery charging circuit 114 which reside within a central housing 100c (in FIGS. 1G and 1H) or 100d ( in FIG. 1I). Each of the brain neuromodulation systems 10g, 10h, and 10i, further includes an external charging system 94.

The external charging system 94 can wirelessly transfer power signals to the power receiver/charging circuit 114. The power receiver/charging circuit 114 converts electromagnetic signals received from the external charging system 94 into a DC voltage for charging the rechargeable power source 112. The external charging system 94 can also include a telemetry circuit to facilitate the communication of control signals and status information between the external charging system 94 and the central housing 100c or 10d.

In FIG. 1G, the central housing 100c includes, carries, or incorporates the lens module 212 having the lens and the optical window, where the lens can include a single or multiple lens elements, and where the optical window may be an integral portion of one or more lenses. FIG. 2J is an illustration of a representative embodiment of the BNS 10g of FIG. 1G, where the central housing 100c is positioned, secured, or affixed in the opening within the patient's skull 1010 to provide neuroillumination to one or more cortical targets.

FIGS. 1H and 1I depict BNS embodiments 10h and 10i having a central housing 100c or 100d and one or more IDUs 200a or 200c. In a manner analogous to that described above for other embodiments, any given rechargeable BNS can include one or more IDUs, each of which can be identical or different in structure and/or specific function. FIG. 2K is a representative illustration of the BNS 10h configured to provide cortical illumination, and FIG. 2L is a representative illustration of the BNS 10h configured to apply subcortical (e.g., deep brain) illumination, in a manner analogous to that previously described for other BNS embodiments. FIG. 2M is a representative illustration of the BNS 10i configured to apply optical signals to subcortical neural targets.

As an alternative or in addition to the foregoing, a BNS of the present disclosure can be RF powered. FIG. 1J is a simplified block diagram of an embodiment of a RF powered BNS 10j of the present disclosure. Relative to other Figures described herein, like or analogous reference numbers indicate like, generally equivalent, corresponding, or analogous elements. The BNS 10j includes an implantable or implanted central housing 100e and an external RF power transfer and diagnostic system 96. The central housing 100e can include an RF power transfer receiver circuit 118, a power management unit 117, the light source driver circuit 140, the light source 142, and the lens module 212.

The circuitry within the central housing 100e relies upon an externally generated RF power signal to induce a voltage and/or current within the RF power transfer receiver circuit 118, which accordingly generates or outputs a DC voltage that can power the elements within the central housing 100e. The power management unit 117 and/or the RF power transfer receiver circuit 118 can include one or more capacitors for storing limited quantities of electrical charge, thereby facilitating implanted device operation for a limited time in the absence of the external RF power signal. The central housing 100e of the RF powered BNS 10j can be smaller than that of the battery powered brain neuromodulation systems disclosed above.

In certain embodiments, the power management unit 117 can include a state machine and a memory (e.g., a set of registers). Depending upon embodiment details, the external RF power signal can be modulated to carry information, such as parameters corresponding to a treatment regiment. In such embodiments, the RF power transfer receiver circuit 118 demodulates or decodes the external RF power signal, and transfers such information to the power supply circuit 117.

The light source driver circuit 140 and the light source 142 operate in a manner analogous to that previously described to provide an appropriate voltage and/or current level to energize the light source 142 when the external RF power signal is present (or when sufficient stored capacitive energy exists), such that the light source 142 can be driven or cycled in accordance with a desired set of ON and OFF states. The light source 142 can include one or more light emitting elements. In various embodiments, the light source 142 includes at least one light emitting diode (LED), but the light source 142 can be essentially any type of device that emits light having an appropriate wavelength and intensity, and which is of small size, low power consumption, and low head generation. The wavelength of light output by the light source 142 can be identical to that described above.

The RF power transfer and diagnostic system 96 is typically used by the patient. In both battery-powered and RF-powered devices, it is suitable for an external device to be able to interrogate the implanted device to determine if the light source is functioning properly. Thus, in certain embodiments (e.g., depending upon embodiment aspects such as overall size and/or power consumption), the central housing can include a circuit to monitor the condition of the light source, and a telemetry circuit in a manner analogous to that previously described. In RF-powered implanted devices that omit a circuit to monitor the condition of the light source and a telemetry circuit, information regarding the condition of the light source can be ascertained by including a circuit in the external RF power transfer and diagnostic system to monitor a level of reflected power. For example, if the implanted light source stops functioning, the amount of power consumed by the implanted device will change, causing a change in reflected power that is indicative of a non-functioning light source.

FIG. 2J illustrates a representative cortical illumination configuration for the BNS 10g of FIG. 1G and of the BNS 10j of FIG. 1J, in which the central housing 100c/100e is implanted in the opening in the patient's skull 1010. In some embodiments, the central housing 100c/100e can be mounted, secured, or fastened to the skull 1010 (e.g., mounted in a burr hole), while in other embodiments, the central housing 100c/100e can be implanted or positioned just beneath the skull 1010 or underneath a skull plug (not shown) that at least partially fills a surgical opening in the skull 1010. In FIG. 2J, the central housing 100c/100e is positioned relative to one or more cortical neural targets, such that light emitted from the lens module 212 is directed into such cortical targets.

FIG. 2N illustrates a representative subcortical illumination configuration of the BNS 10j of FIG. 1J, in which the central housing 100e is implanted at a subcortical location in the brain to apply optical illumination to particular subcortical and/or deep brain neural targets or structures.

FIGS. 1K and IL are simplified block diagrams of additional RF powered embodiments of a BNS 10k and 10l, respectively of the present disclosure. Relative to other Figures described herein, like or analogous reference numbers indicate like, generally equivalent, corresponding, or analogous elements. As shown in FIG. 1K, in some embodiments an RF powered BNS 10k can include one or more types of IDUs 200a, one or more of which can be structurally and/or functionally analogous to other embodiments described above. In the embodiment shown, a central housing 100f includes the light source 142, and optionally includes the lens 144. The IDU 200a includes the optical fiber bundle 202 and possibly the terminal or distal lens 220, in a manner analogous to that described above with reference to FIG. 1A. FIGS. 2K and 2L respectively illustrate representative cortical illumination and subcortical illumination embodiments of the BNS 10k of FIG. 1K.

As shown in FIG. 1L, the RF powered BNS 101 can include one or more light sources 142 that are external to a central housing 100g. In such embodiments, the BNS 101 can include a set of identical or different IDUs 200d, 200e having particular types of illumination housings 230a, 230b, for instance, in a manner depicted in FIG. 1L. FIG. 2M illustrates a representative subcortical illumination embodiment of the BNS 101 of FIG. 1L.

### Cortical and Subcortical Illumination Brain Neuromodulation Systems

Various non-rechargeable, rechargeable, and/or RF powered devices of the present disclosure can provide both cortical and subcortical illumination to one or more neural targets in a simultaneous or sequenced manner. FIG. 2O illustrates a representative BNS 10o configured to deliver both cortical and subcortical neuroillumination. The BNS 10o includes at least one cortical neuroillumination delivery device or neuroillumination probe 280, and at least one subcortical neuroillumination delivery device or neuroillumination probe 290.

In a particular context, a given categorical type of neuroillumination can be defined based upon an anatomical or neural location at, to, or from which illumination is applied. In such a context, “cortical neuroillumination” can refer to illumination that is applied from an intracranial location (i.e., a location beneath the cranium or skull 1010) that is external to the brain, where the intracranial location is proximate or generally proximate to the skull 1010 and upon, adjacent to, over, above, or near a cortical tissue boundary defined by an exterior or outer surface or region of the brain. Cortical neuroillumination can be epidural or subdural.

The cerebral cortex or neocortex occupies an outermost portion or region of the brain, and generally has a thickness of approximately 1.0-4.5 mm (depending upon factors such as brain location and patient condition or age). As such, “intracortical neuroillumination” can refer to illumination that is applied by a device that resides within the brain's outer layer of cortical tissue (i.e., within or approximately within the aforementioned 1-3 mm thickness). Furthermore, “subcortical neuroillumination” can refer to illumination that is applied from a location within the brain that is below or beneath the brain's outer layer of cortical tissue that resides proximate to the skull 1010.

From a neuroanatomical perspective, particular cortical tissue can reside at a location that is substantially interior to the cranium or skull 1010, such as a mid-brain or deep brain location. Such cortical tissue can include, for example, the cingulate cortex. Thus, “mid-brain neuroillumination” and “deep brain neuroillumination” can refer to particular categorical types of neuroillumination, where neural tissue or neural target overlap can exist between certain categorical types of neuroillumination.

A neuroillumination delivery device 280, 290 includes at least one site, location, or position at or from which light emanates or is emitted. A neuroillumination delivery device 280, 290 can include a distal light emission site 208a (e.g., corresponding to a distal tip of a tip emitting optical fiber bundle 202) and/or one or more side emission sites 208b (e.g., corresponding to a particular portion, segment, or section of a side-emitting optical fiber bundle 202), such that a neuroillumination delivery device 280, 290 can be configured to illuminate a set of neural targets in accordance with one or more separate neural target locations and/or categorical types of neuroillumination. In an embodiment, an as-manufactured distance or separation between particular light emission sites 208a, 208b can be defined in accordance with an approximate expected separation or distance between a first neural target or neural target type and a second neural target or neural target type. In a representative embodiment, an as-manufactured distance between a first light emission site 208a, 208b and a second light emission site 208a, 208b can be based upon an expected separation distance between a portion of the motor cortex, such as the “hand knob” within the precentral gyrus, and a portion of the thalamus, where the expected separation distance can be a calculated average distance corresponding to neural imaging data for a patient population. The definition of such a separation distance can further involve a consideration of an expected or typical device implantation trajectory. In another representative embodiment, an as-manufactured separation distance or relative positioning between a first light emission site 208a, 208b and a second light emission site 208a, 208b can be based upon an expected distance between a prefrontal or orbitofrontal cortex neural target location and a cingulate cortex neural target location, considering an expected device implantation trajectory. Separate light emission sites 208a, 208b can be dimensioned to have a length or span that can accommodate certain patient-to-patient and implantation trajectory variations. In an embodiment, a single neuroillumination delivery device 290 can be configured to apply two or more distinct or distinguishable forms of intracortical, subcortical, mid-brain, and/or deep brain neuroillumination to a plurality of neural targets in a variety of manners.

A given light emission site 208a, 208b can include or correspond to a particular light emitting element, such as a set of optical fibers or an LED. Accordingly, a given light emission site 208a, 208b can be categorized as an active light emission site if light is generated at, within, or adjacent to the light emission site; or a passive light emission site if light propagates or is transferred to and through or away from the light emission site.

In an embodiment, a BNS 10o can include one or more neuroillumination delivery devices 280, 290 configured to provide or apply cortical, intracortical, or subcortical, mid-brain, and/or deep brain illumination at one or more times. Such a BNS 10o can illuminate multiple neural targets simultaneously or sequentially, possibly in a programmable or selectable manner (e.g., in accordance with or as enabled by a set of program instructions stored within a computer readable medium such as a memory). A particular BNS configuration can depend upon patient symptoms or state, a type of neurologic function or dysfunction under consideration, and/or device embodiment details.

A given neuroillumination delivery device 280, 290 can include optical elements (e.g., one or more optical fiber bundles 202, a lens 220, and/or a lens module 212) only, or both optical and electrical elements (e.g., one or more light sources 142) in a manner analogous to that described above, depending upon whether a primary or central housing includes or excludes light emitting devices.

Brain Neuromodulation Systems with Electrical Stimulation and/or Sensing

In some embodiments, a BNS can also include one or more structures, devices, or elements directed toward the application of electrical stimulation signals to particular neural targets. Such neural targets can include cortical, subcortical, spinal column, or other neural populations. Neural targets to which electrical stimulation is applied or directed can be identical to, or distinct from, neural targets to which optical illumination is applied or directed. Furthermore, in some embodiments, the BNS can include one or more sensing elements, which can be configured to capture, generate, and/or transfer signals corresponding to neuroelectric activity, cerebral bloodflow, a blood oxygenation level, temperature (e.g., a temperature at, in, or proximate to an anatomical location, for instance, a neural target or a particular brain location), or the presence, absence, or a relative level of an ionic species (e.g., Hydrogen ion concentration, or pH) or chemical substance.

In general, a particular type of sensing device or element can correspond to a particular type of physiologic parameter, characteristic, state, or condition. A set of sensed signals or data can be defined as a sensed parameter that directly or indirectly represents, indicates, or defines a value for a physiologic parameter, characteristic, state, or condition at a specific time or with respect to a given time interval (e.g., a maximum, minimum, and/or average bodily temperature during a 1 hour period). Sensing elements can be implanted or positioned at or relative to particular cortical, subcortical, spinal column, or other neural populations. Neural targets to which sensing operations are directed can be identical to, or distinct from, neural targets to which optical illumination is applied or directed.

FIGS. 1M and 1N are simplified block diagrams of two embodiments of brain neuromodulation systems 10m and 10n, of the present disclosure. Relative to other Figures described herein, like reference numbers indicate like, generally equivalent, corresponding, or analogous elements. In particular embodiments, the BNS 10m and 10n includes a central housing 100h and 100i, having an electrical stimulation unit 160 and/or a sensing unit 170.

The electrical stimulation unit 160 can be coupled to one or more electrode devices 260, where any given electrode device 260 can include one or more deep brain, subcortical, penetrating or needle type, or surface electrode device. In an embodiment, the electrical stimulation unit 160 generates and outputs electrical pulses or waveforms (e.g., a charge balanced biphasic waveform) under the direction of program instructions stored in the memory 126. In such an embodiment, various electrical stimulation parameters can be specified, including current or voltage level, pulse repetition frequency, a pulse phase temporal width, electrical signal polarity (e.g., bipolar, anodal unipolar, or cathodal unipolar), and possibly a pulse or waveform modulation function.

The sensing unit 170 can be coupled to one or more sensing devices 270, and can initiate, perform, direct, or manage sensing operations such as the detection and/or analysis of signals received from particular sensing devices 270. Various sensing operations can occur in an automated or semi-automated manner in accordance with, under the direction of, or in response to a set of program instructions that reside within a memory.

In an embodiment, the sensing unit 170 includes memory (e.g., a register set) for storing signals received from sensing devices 270, and a state machine or other type of processing unit or processor. Depending upon embodiment details, the sensing device 270 can include one or more electrode devices 260, for instance, a set of electrodes configured to detect or measure neuroelectric (e.g., ECOG) activity. The sensing device 270 can additionally or alternatively include an electrical substance sensor, such as a ChemFET, that is configured to sense pH or the presence or level of a particular chemical substance; a temperature sensor, such as a thermocouple or a solid state (e.g., Schottky barrier) based device; and/or other types of devices, as further described below.

The sensing unit 170 can activate or communicate with particular sensing devices 270 (e.g., a temperature sensor) at periodic intervals (e.g., once per hour, or at particular times of day, or twice or three times daily; or before, during, and/or after electrical stimulation operations), or in response to signals detected or generated by other sensing elements or devices 270 such as an electrode configured to detect neuroelectric activity, and/or a pH sensor. Thus, in an embodiment, the sensing unit 170 can activate, communicate with, or query different sets or subsets of sensing devices 270 at different times to acquire or generate corresponding or associated sets of sensed parameters.

In an embodiment, the sensing unit 170 can query or poll a sensing device 270, or access a set of memory locations at which signals or data acquired or transferred by sensing devices 270 is stored. The sensing unit 170 can determine whether a particular sensed parameter falls within a predetermined (e.g., acceptable or unacceptable, or safe or unsafe) range, or is above a predetermined upper threshold or below a predetermined lower threshold. A predetermined sensed parameter range, upper threshold, or lower threshold can be programmably specified, and an evaluation of a sensed parameter relative to the aforementioned range or thresholds can occur in accordance with a set of program instructions. A sensed parameter that falls outside of a predetermined range (e.g., above an upper threshold value or below a lower threshold value) can indicate the existence or a likelihood of an oxidative stress condition, a neurodegeneration condition, a neuronal damage condition, and/or a dysfunctional metabolic condition (e.g., reduced or low mitochondrial metabolism) within or proximate to a neural target, or an expectation that one or more such conditions has occurred, is occurring, or will occur. Thus, a sensed parameter that falls outside of a predetermined range can correspond to an oxidative stress, neurodegeneration, neuronal damage, or abnormal metabolic (e.g., hypometabolic or hypermetabolic) condition within a neural target under consideration.

In response to an evaluation or analysis of one or more sensed parameters, the sensing unit 170 or the system control unit can initiate a neuroillumination intervention process directed toward reducing an effect, or preventing or reducing a likelihood, of oxidative stress, neurodegeneration, neuronal damage, or metabolic dysfunction within one or more neural targets. The neuroillumination intervention process can involve the application of light to a set of neural targets (e.g., as described herein in relation to various embodiments of the disclosure) during an intervention period. For instance, in response to one or more sensed temperature values that exceed an upper temperature threshold or limit, or which fall below a lower temperature threshold, neuroillumination can be provided to certain neural targets at or near one or more brain locations that correspond to the sensed temperature value(s).

In an embodiment, the sensing device 270 includes a set of photodetectors (e.g., a photodiode), such that the sensing device 270 can detect optical signals corresponding to particular wavelengths of light. In an embodiment, the sensing unit 170 uses sensed or detected optical signals to determine whether an amount or intensity of illumination proximate to, near, or traveling through a given neural target is at a desired level (e.g., approximately 8-12, or approximately 10 mW/cm²). If not, the sensing unit 170 can generate a signal that the light source driver circuit 140 can utilize to adjust a light intensity output by the light source 142.

In an embodiment, the sensing device 270 can include or be coupled (e.g., by the optical fiber bundle 202) to one or more optical emission devices or light sources (e.g., LEDs) that emit light at particular wavelengths to facilitate near infrared spectroscopy (NIRS) measurements. Alternatively, one or more optical sources or LEDs for NIRS measurements can be included as part of the light source 142 within the central housing 100h or the IDU 200e.

In an embodiment, the BNS 10m or 10n, applies and senses or detects changes in near infrared light absorption at particular times to monitor or measure at least one of an oxyhemoglobin, a deoxyhemoglobin, and/or a cytochrome oxidase condition (e.g., a redox state), in a manner identical or analogous to that described by Hoshi et al. in “Redox behavior of cytochrome oxidase in the rat brain measured by near-infrared spectroscopy,” Journal of Applied Physiology 83: 1842-1848, 1997, which is incorporated herein by reference in its entirety. A specific or a relative oxyhemoglobin, deoxyhemoglobin, and/or cytochrome oxidation condition can indicate a metabolic state associated with a neural target, and/or a level of oxidative stress that the neural target has experienced or is experiencing.

In an embodiment, the BNS 10m or 10n is configured to perform NIRS measurements that can include multiple near infrared optical sources, such as LEDs that output light at approximately 700, 730, 750, and 805 nm wavelengths to facilitate the detection or determination of wavelength dependent changes (e.g., near infrared absorption changes) corresponding to an oxyhemoglobin, deoxyhemoglobin, and/or cytochrome oxidase state associated with target neural tissue. One or more optical sources used for therapeutic or neuroprotective illumination of neural tissue, such as an LED that emits light primarily at a center wavelength of 670 nm, and/or an LED that emits light primarily at a center wavelength of 820 nm, can also be used to facilitate NIRS measurements.

The sensing unit 170 and/or the sensor(s) 270 can include a set of devices configured to perform (NIRS) operations or measurements. In an embodiment, a set of sensors 270 and particular sensing unit circuitry that together are configured to perform NIRS operations or measurements can be defined as a NIRS unit. NIRS-related sensors 270 can be activated to perform sensing operations or measurements that can indicate a cytochrome oxidase condition within a neural target at or adjacent or proximate to which a sensing device 270 resides. For instance, the sensing unit 170 can detect or determine an approximate cytochrome oxidase redox state, or a change over time in an optical parameter corresponding to a redox state. A cytochrome oxidase redox state, or a change in such a state, can indicate or correspond to a level or extent of oxidative stress or damage that neural tissue has undergone and/or is experiencing.

Based upon one or more sensing operations directed toward measuring or estimating a cytochrome oxidase redox state or other condition (e.g., a hemoglobin oxygenation state), program instructions executed by the programmable processor 122 can issue commands to activate, deactivate, or adjust the light emitted by the one or more light sources 142. For example, in the event that a NIRS measurement indicates that a cytochrome oxidase redox and/or other biological state at or proximate to a neural target corresponds to an increased or potentially detrimental level of oxidative stress, the BNS 10m or 10n can activate the one or more light sources 142 to provide optical signals to the neural target under consideration, and possibly additional neural targets. In an embodiment, such optical signals can be delivered at a power level of approximately 8-12 mW/cm2 for about 3-12 minutes in order to enhance a likelihood that the applied light signals provide or give rise to neuroprotective effects in the neural target(s). Furthermore, additional optical signals can be automatically delivered to one or more of such neural targets after a predetermined or selectable time interval, for example, approximately 1 hour, 4 hours, 8 hours, 12 hours, or 24 or more hours.

FIG. 2P illustrates a representative embodiment of the BNS 10m or 10n configured for both neuroillumination and electrical stimulation operations. In the embodiment shown, the BNS 10m or 10n includes at least one optical and electrical signal delivery probe, paddle, or device 250, which carries or includes the optical fiber bundle 202 and at least one set of electrodes or electrical contacts 252.

In an embodiment, the optical and electrical signal delivery probe 250 can include an elongated insulating shaft of member that is hollow, in which the optical fiber bundle 202 is positioned. The elongated shaft can be open at a distal end such that a tip of the optical fiber bundle 202 can illuminate a neural target at a distal end of the probe 250. Additionally or alternatively, the elongated shaft can have one or more openings or windows along its length, to facilitate the delivery of light to neural targets adjacent or proximate to particular positions along the length of the probe 250 by a side emitting optical fiber bundle 202.

In an embodiment, the optical fiber bundle 202 can form a hollow cylindrical structure, in and through which an electrically insulated lead line is positioned. The lead line encloses or surrounds one or more electrically conductive lead wires. The set of electrodes or electrical contacts 252 residing upon an outer surface of the optical fiber bundle 202 (e.g., near a distal tip, and/or at one or more positions along the optical fiber bundle's length) can be coupled to particular lead wires to facilitate the delivery of electrical signals to one or more neural targets.

In an embodiment, an illumination paddle 204a, 204b, 204c, such as shown in FIGS. 8A through 8C, can include a set of planar electrical contacts and lead wires, in a manner analogous to that for chronically implanted cortical and/or spinal column stimulation (SCS) electrodes.

FIG. 2Q illustrates a representative embodiment of the BNS 10m or 10n configured for neuroillumination and sensing operations. In certain embodiments, the BNS 10m and 10n include at least one neuroillumination and sensing probe, paddle, or device 256 that carries or includes the optical fiber bundle 202 and the at least one set of sensors or sensing devices 270. Depending upon embodiment details, a neuroillumination and sensing probe 256 can have a structure that is generally analogous to that described above for an optical and electrical signal delivery probe 250, particularly in the event that one or more sensing devices 270 are electrode-based.

In addition to the foregoing, in some embodiments a BNS can be configured for optical signal as well as electrical signal delivery, and one, some, or each of optical signal sensing, electrical signal sensing, and chemical species or substance sensing. Such a BNS can include a multi-modal neurotherapy probe, paddle, or device that accordingly carries or includes optical illumination, electrical stimulation, optical sensing, electrical sensing, and/or chemical or substance sensing devices.

### Intraventricular Illumination Brain Neuromodulation Systems

The irradiation or illumination of one or more neural targets can additionally or alternatively be accomplished using systems, apparatus, and/or devices that include elements implanted into one or more cerebral ventricles. The implantation of portions of a BNS into a cerebral ventricle can facilitate less invasive neurosurgical implantation procedures in view of positioning one or more neuroillumination devices relative to certain subcortical or deep brain targets. In various embodiments, a BNS of the present disclosure can be implanted intraventricularly, and can be powered by a primary battery, a rechargeable battery, or RF energy.

An intraventricular and/or transventricular BNS disclosed herein can include an elongated neuroillumination probe having one or more straight, generally straight, curved, and/or contoured portions or segments. The neuroillumination probe can include a set of light emission sites, locations, sections, regions, or structures that are positioned relative to such neuroillumination probe portions or segments. The light emission site can include one or more portions of an optical fiber bundle, such as a distal tip or a side emitting portion of the optical fiber bundle. Additionally or alternatively, a neuroillumination probe and/or a light emission site can include one or more lens elements and/or optical windows, which can control optical beam size, shape, and/or spread characteristics, which can facilitate the preferential emanation of light in a specific direction, along an intended or particular optical path, or toward or through particular neural targets relative to other neural targets. A light emission site can further include or correspond to a light source such as an LED. Light emitted at a light emission site is preferentially directed into a particular (e.g., a predetermined) neural target according to the construction of the neuroillumination probe and the specific ventricular location(s) at or in which the probe is implanted, positioned, or maintained. Such illumination can emanate from an intraventricular location, and transventricularly propagate across or through a ventricular boundary to an intended neural target.

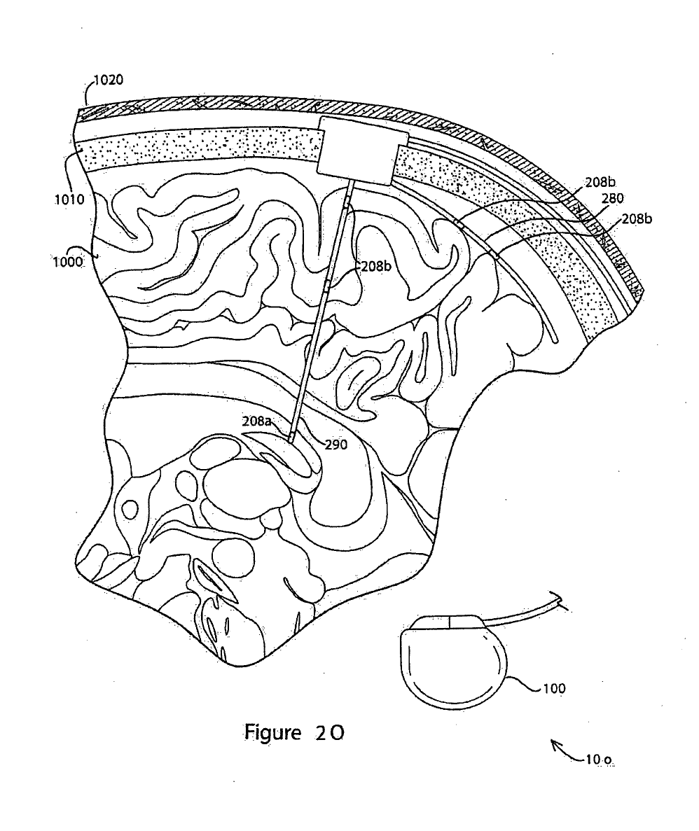

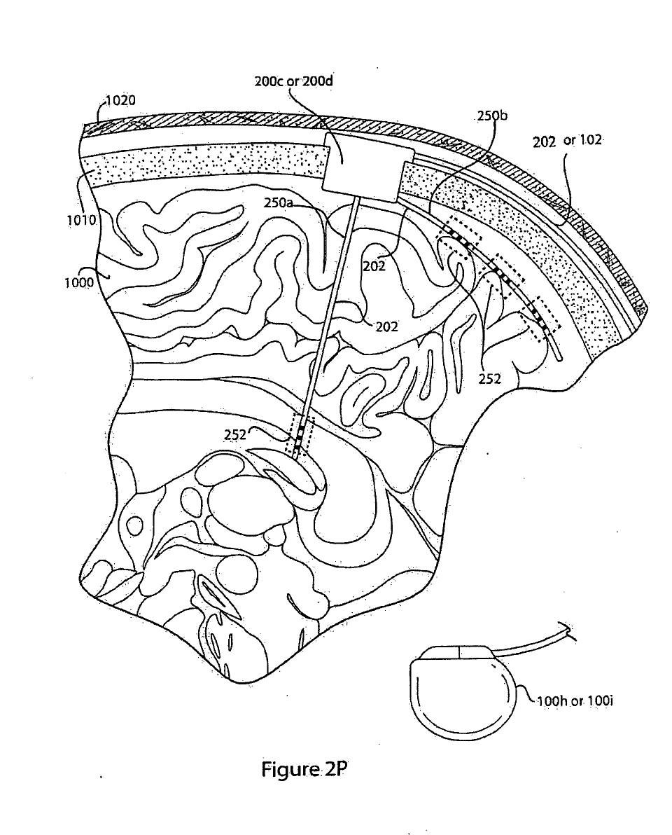

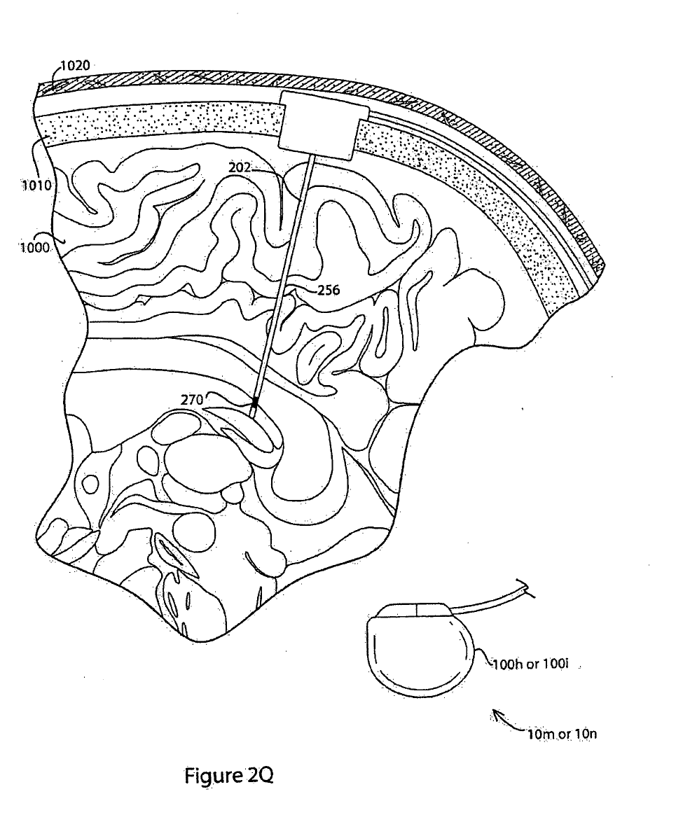

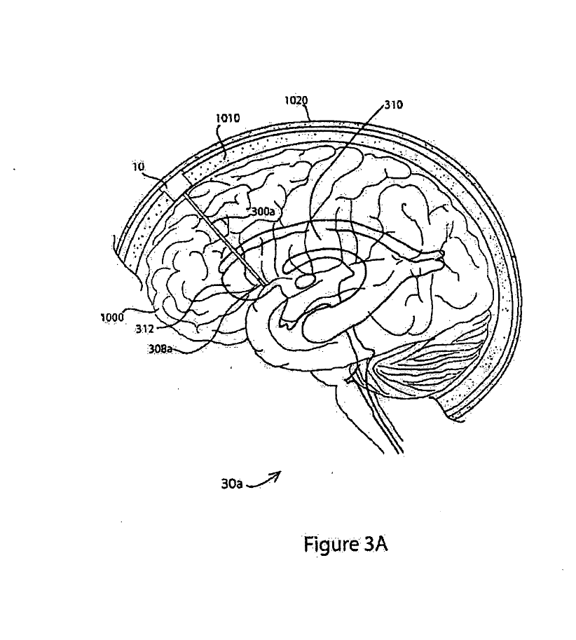

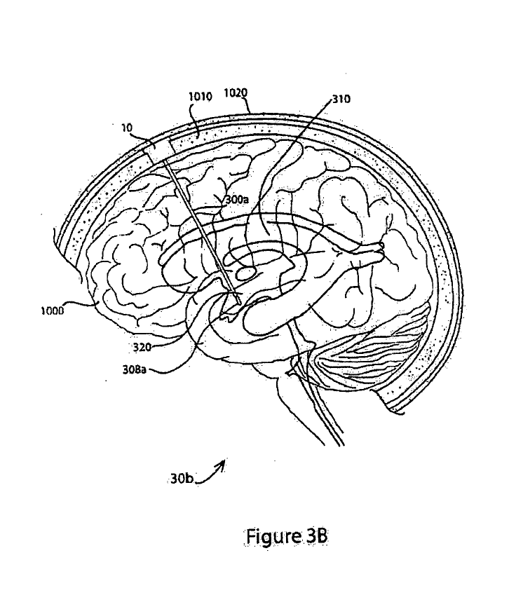

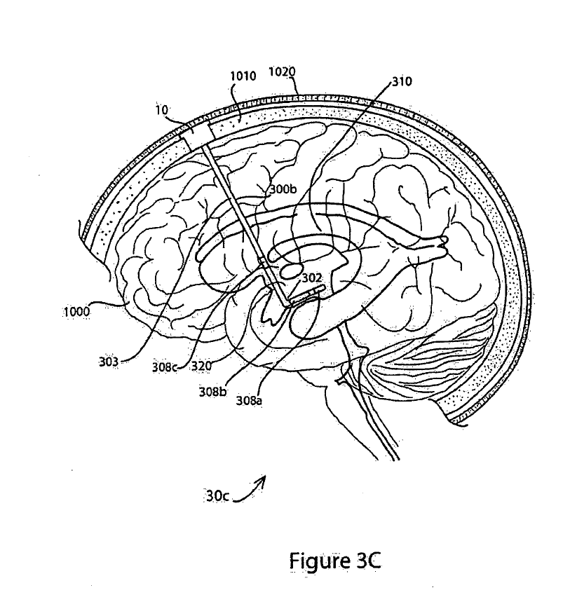

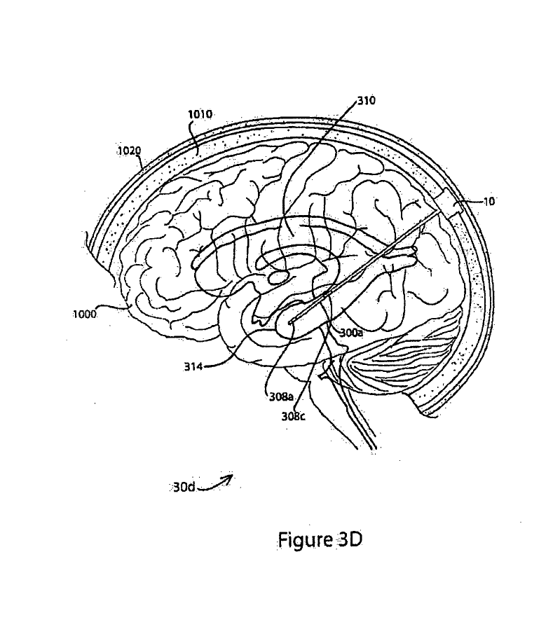

FIG. 3A shows a cross sectional anatomical illustration of an embodiment of an intraventricular BNS 30a according to the present disclosure. The intraventricular BNS 30a includes BNS 10 having a neuroillumination probe 300a implanted into, proximate to, or generally near an anterior horn 312 of a lateral ventricle 310. In general, the neuroillumination probe 300a can be essentially any type of device, structure, member, or probe having one or more optical signal delivery elements, portions, segments, or regions. For instance, the neuroillumination probe 300a can include optical fiber bundle that emits optical signals at a distal tip, in accordance with various BNS embodiments described herein. In such an embodiment, the distal tip can form a light emission site 308a. An intraventricular system, apparatus, or device in accordance with particular embodiments of the disclosure can additionally or alternatively include a side emitting optical fiber bundle, and one or more additional light emission sites, as detailed in relation to other representative BNS embodiments described below.

The implantation of a neuroillumination probe 300a into the anterior horn 312 of the lateral ventricle 310 facilitates the optical irradiation of particular neural targets, such as the nucleus accumbens, one or more of portions of the frontal lobe such as the orbitofrontal cortex; portions of the cingulate gyrus; and one or more neural targets corresponding or expected to correspond to particular Brodmann areas, such as Brodmann area 9, Brodmann area 10, Brodmann area 14, Brodmann area 25, and Brodmann area 46. Based upon a patient condition or disorder under consideration and a set of corresponding or associated neural targets, a neurosurgeon can choose a particular set of anatomical reference points and/or neuroanatomical coordinates as part of an intraventricular implantation procedure to ensure that the neuroillumination probe 300a is appropriately positioned within a ventricle relative to one or more neural targets to be illuminated.

FIG. 3B shows a cross sectional anatomical illustration of an embodiment of an intraventricular BNS 30b of the present disclosure. The intraventricular BNS 30b includes BNS 10 having the straight or generally straight neuroillumination probe 300a implanted into a third ventricle 320. The straight neuroillumination probe 300a can include a light emission site 308a at a distal tip.

FIG. 3C shows a cross sectional anatomical illustration of an embodiment of an intraventricular BNS 30c of the present disclosure, which includes BNS 10 having a shaped or contoured neuroillumination probe 300b implanted into the third ventricle 320. The shaped neuroillumination probe 300b includes a distal segment 302 and a generally straight proximal or elongated segment 303. In an embodiment, the distal segment 302 can have a different length, diameter, curvature, or geometric shape or design than the proximal segment 303, possibly in a manner that generally follows or conforms to an anatomical boundary such as a portion of the third ventricle 320. The distal segment 302 can include or carry a side emitting optical fiber bundle, such that one or more light emission sites 308b reside or extend across a portion of the distal segment 302. Additionally or alternatively, the distal segment 302 can terminate at a tip that provides or forms a light emission site 308a. Also, the elongated segment 303 can include or carry one or more light emission sites 308 at particular positions along its length.

The intraventricular brain neuromodulation systems shown in FIGS. 3B and 3C can facilitate the illumination of neural targets such as the hypothalamus; the pineal region; the midbrain, including portions of the basal ganglia, the substantia nigra and/or the thalamus; the subthalamus; the nucleus of Meynert; portions of the optic tract and/or optic nerve; and/or other targets.

FIG. 3D shows a cross sectional anatomical illustration of an embodiment of an intraventricular BNS 30d of the present disclosure, which includes BNS 10 having the neuroillumination probe 300a implanted into an inferior or temporal horn 314 of the lateral ventricle 310. The BNS 10 can include a straight or generally straight elongated neuroillumination probe 300a. The neuroillumination probe 300a can have a light emission site 308a at a distal tip, and possibly one or more light emission sites 308c along particular portions of the probe's length. The neuroillumination probe 300a implanted into the inferior horn 314 of the lateral ventricle 310 can deliver optical signals to neural targets such as the hippocampus, the amygdala, and the amygdaloid body.

Depending upon a neuroillumination probe's ventricular implantation trajectory, a neuroillumination probe that includes multiple light emission sites 308a, 308c can be implanted or positioned such that one or more light emission sites 308a, 308c reside within a ventricle, and one or more light emission sites 308c reside external to the ventricle. Thus, depending upon neuroillumination probe embodiment details and an implantation trajectory, an intraventricularly implanted neuroillumination probe may provide extraventricular illumination in addition to transventricular illumination to a set of neural targets.

FIG. 3E shows a cross sectional anatomical illustration of an embodiment of an intraventricular BNS 30e of the present disclosure. The intraventricular BNS 30e includes BNS 10 having a shaped or contoured neuroillumination probe 300c implanted along an anterioposterior span of the lateral ventricle 310. In an embodiment, such a neuroillumination probe 300c extends throughout or along a substantial anterior-posterior extent of the lateral ventricle 310. The neuroillumination probe 300c can have a curved, contoured, or shaped geometry, portion, or section 304 (e.g., a distal portion) that generally follows, matches, or conforms to an expected or imaged (e.g., using a CT, MRI, or MR angiography (MRA) system) ventricular boundary or curvature within the ventricular region in which the neuroillumination probe 300c is implanted. A curved, contoured, or shaped portion 304 of the neuroillumination probe 300c can have a different length, diameter, curvature, or geometric shape or design than another portion (e.g., a proximal segment) of the probe 300c.

The neuroillumination probe 300c can carry or include one or more side and/or tip emitting optical fiber bundles, such that the neuroillumination probe 300c has one or more light emission sites 308c along its length. Particular light emission sites 308c can be positioned along or reside upon the probe's contoured section 304. The neuroillumination probe 300c can also include a light emission site 308a at a distal tip. In certain embodiments, particular subsets of light emission sites 308a, 308c can emit light at one or more times, possibly in accordance with a set of program instructions that manage the illumination of certain neural targets at particular times, and/or based upon or in response to the sensing of signals and/or substances by one or more sensing devices 270 coupled to the sensing unit 170. In such embodiments, light sources 142 that are optically coupled to particular optical fiber bundles 202 or particular fiber subsets within an optical fiber bundle 202 can be activated in one or more manners (e.g., in a predetermined or as-needed activation sequence) at particular times, and/or based upon or in response to particular detected patient states, conditions, or events.

The neuroillumination probe 300c shown in FIG. 3E can deliver optical signals to multiple neural targets at neuroanatomical locations that are adjacent or proximate to or near a given posterior—anterior extent of the lateral ventricle 310. Such neural targets include particular portions of the caudate nucleus, the cingulate gyrus, the frontal lobe (e.g., areas of the orbitofrontal cortex), the parietal lobe, and/or other neural targets.

In other embodiments of intraventricular or transventricular neuroillumination systems of the present disclosure, the neuroillumination probe can carry or include one or more LEDs or optical light sources in addition or as an alternative to carrying one or more optical fiber bundles. A light emission site in such an embodiment can correspond to a neuroillumination probe position or location at which the LED, the optical fiber bundle, the lens, and/or the optical window reside. LEDs carried within a neuroillumination probe can be electrically coupled to circuitry such as the light source driver that resides within the central housing.

### Representative Catheter or Shunt Illumination Systems and Devices

In addition to the embodiments described above for brain neuromodulation systems, the teachings of the present disclosure can be applied to various embodiments of other types of devices that are implanted in the brain and/or other anatomical locations. For instance, one or more light emitting and/or light transmitting devices or elements can be incorporated into a shunt catheter structure to facilitate the optical irradiation or illumination of target tissue at, near, proximate to, or surrounding portions of the catheter structure. Such a shunt catheter structure can also include one or more sensing devices for detecting particular types of signals, chemical compositions, species, or substances.

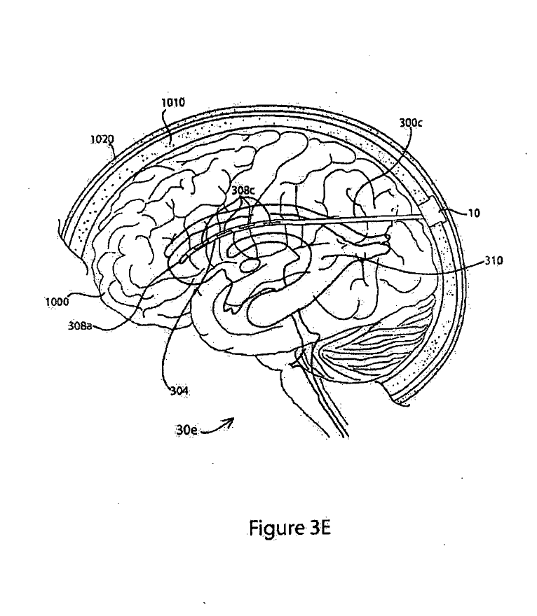

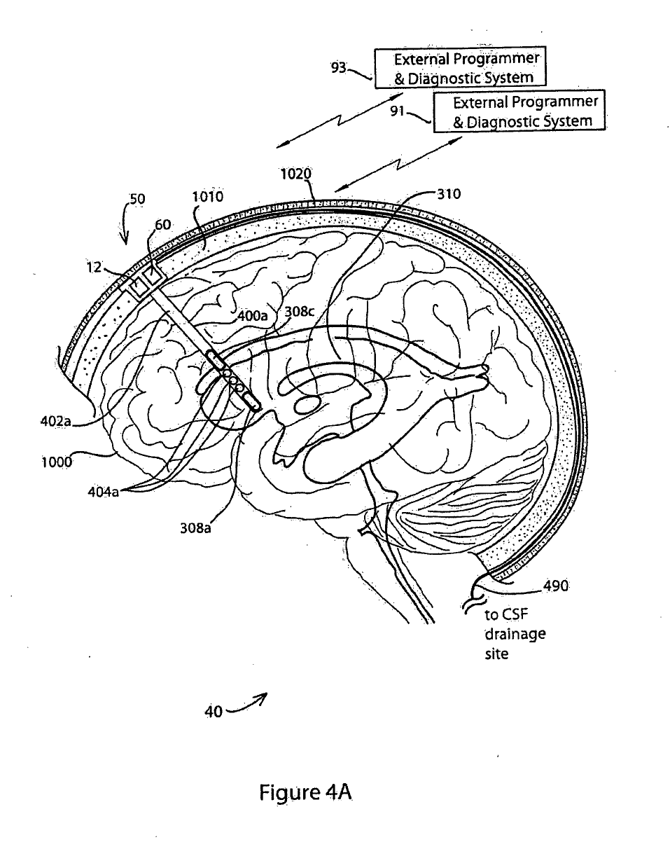

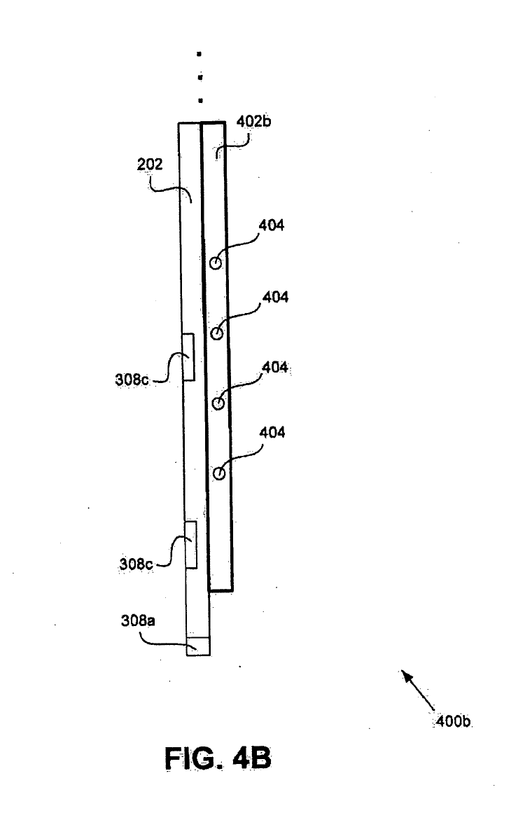

FIG. 4A is a cross sectional anatomical illustration of a ventricular illumination and drainage system (VIDS) 40 implanted into the lateral ventricle 310 according to an embodiment of the present disclosure. In certain embodiments, the VIDS 40 includes an implanted illumination and drainage controller 50, an implanted illumination shunt 400a, an implanted distal drainage catheter 490, and an external programmer and diagnostic system 91. The VIDS 40 can optionally include a patient control unit 93. In an embodiment, the illumination and drainage controller 50 includes a housing 52 (e.g., a hermetically sealed, biocompatible Titanium housing) in which a BNS control module 12 and a shunt control module 60 reside.

The BNS control module 12 manages or directs the generation and distribution of optical signals to neural targets. The BNS control module 12 can include various elements for controlling, generating, and distributing or applying optical signals to neural targets, such as elements previously described above with reference to particular embodiments of BNS central housings 100 and IDUs 200. The shunt control module 60 can include a one way valve, a fluid reservoir, and a valve controller that establishes or regulates a fluid pressure or drainage rate, possibly in accordance with one or more shunt control parameters corresponding to a fluid pressure threshold or a maximum fluid flow rate. In an alternate embodiment, a one way valve can be carried by the illumination shunt 400a, the fluid reservoir, or the distal drainage catheter 490. The illumination shunt 400a, the one way valve, the fluid reservoir, and the distal drainage catheter 490 are coupled or connected in a manner that facilitates the transfer, flow, or extraction of cerebrospinal fluid from a ventricle to another anatomical location. Devices or elements that are coupled in such a manner are defined herein to be in fluid communication. The BNS control module 12 can include a power source, which in certain embodiments can also supply power to the shunt control module 60.

The illumination shunt 400a includes a tube and/or other structure that facilitates fluid transfer or drainage; and one or more devices, elements, and/or structures that generate, transfer, and distribute, propagate, radiate, or emanate optical signals toward or into target tissue. The illumination shunt 400a is coupled, connected, or attached to the illumination and drainage controller housing 52 such that fluid is routed into the shunt control module's 60 fluid reservoir, under the control of a fluid pressure threshold established or maintained by the valve controller lo and the one way valve. The illumination shunt 400a is further coupled or connected to the illumination and drainage controller housing 52 such that optical and/or electrical signal transfer elements are coupled, routed, or connected to appropriate types of optical and/or electrical signal generation and transfer elements, such as those previously described herein that form portions of the neuroillumination systems of the present disclosure.

The external programmer and diagnostic system 91 and the patient control unit 93 are configured for wireless communication with the BNS control module 12 to establish and/or manage optical illumination operations. In certain embodiments, the external programmer and diagnostic system 91 is further configured for wireless communication with the shunt control module 60 to establish one or more shunt control parameters (e.g., a fluid pressure threshold) for fluid drainage. In an embodiment, the implanted illumination and drainage controller 50 includes a telemetry unit that is shared by the BNS control module 12 and the shunt control module 60.

In an embodiment, the illumination shunt 400a includes a hollow elongated tube 402a having a set of openings or holes 404 therein that allow or facilitate fluid transfer. The illumination shunt 400a can further carry or include the optical fiber bundle 202. In particular embodiments, the optical fiber bundle 202 securely resides within and extends along at least a portion of the length of the hollow elongated tube 402a. The optical fiber bundle 202 include a set of tip emitting and/or a set of side emitting optical fibers, such that a distal tip and/or one or more side portions of the optical fiber bundle 202 can form light emission sites 308a, 308c. The hollow elongated tube 402a can include openings or transparent optical windows at particular positions along its length, corresponding to or aligned with optical fiber bundle light emission sites, to facilitate the emission of light into neural targets. In certain embodiments, a single set of openings 404 within the hollow elongated tube 402a provide for both fluid transfer and the passage or propagation of light away from the optical fiber bundle light emission sites 308a, 308c.

FIG. 4B is a schematic illustration of a portion of an illumination shunt 400b according to an embodiment of the present disclosure. The illumination shunt 400b includes optical fiber bundle 202 that is attached or affixed to at least a portion of the exterior of a hollow fluid drainage tube 402b. The fluid drainage tube 402b can include one or more openings 404 that facilitate fluid transfer. Depending upon embodiment details, the fluid drainage tube 402b can be approximately the same overall length as the optical fiber bundle 202, or a different length (e.g., shorter) than the optical fiber bundle 202. The optical fiber bundle 202 can include tip and/or side emission portions that form corresponding light emission sites 308a, 308c. In certain embodiments, the hollow fluid drainage tube 402b can be indented or shaped, such that a portion of the optical fiber bundle 202a is seated in and/or along the indented portion of the hollow fluid drainage tube 402b.

Depending upon embodiment details, the illumination shunt 400 can be implanted into various cerebral ventricles or other anatomical structures. In some embodiments, the illumination shunt 400 can include one or more contoured or shaped portions that generally follow or conform to an expected or actual (e.g., imaged) shape or boundary of an anatomical structure such as a given region of a cerebral ventricle.

In an embodiment, the BNS control module 12 can further include the electrical stimulation unit 160 and/or the sensing unit 170, and the illumination shunt 400 can accordingly include the set of electrode devices 260 and/or the set of sensing devices 270 that are coupled to the electrical stimulation unit 160 and/or the sensing unit 270. In an embodiment, the sensing device 270 can be a pressure detector that can detect or measure a bodily fluid pressure parameter or a set of bodily fluid pressure values. Neuroillumination can be initiated (e.g., at a wavelength of 670 nm, for a period of up to approximately 20-30 minutes, or a period of approximately 5-15 minutes, or approximately 3-12 minutes, at a power level of approximately 5-15 mW/cm²) when one or more detected pressures exceed a given (e.g., predetermined) reference or threshold pressure level or value, or change by more than a predetermined amount or extent relative to a reference pressure value or a previously detected pressure. The sensing device 270 can additionally or alternatively be a flow detector, such that neuroillumination can be initiated based upon a fluid flow condition or state such as an absence or near absence of detectable fluid flow over a predetermined period of time.

The procedures used to implant components to irradiate cortical and/or subcortical targets are similar or analogous to the procedures used to implant chronic electrical cortical stimulation (CS) and Deep Brain Stimulation (DBS) electrodes. These procedures are known in the art and are fairly standardized, but multiple nuances exist, depending on the preferences of the particular surgeon and the particular equipment used. Electrical stimulation-based neuromodulation systems are a known surgical intervention for symptoms of refractory movement disorders such as Parkinson's disease, essential tremor, and dystonia.

In an embodiment, a process for implanting components to irradiate subcortical targets involves the imaging of the intended subcortical structure and the construction of a three-dimensional stereotactic targeting dataset. To accomplish this, a standard three-dimensional coordinate system must be related or registered to the specific three-dimensional anatomy of the patient's brain. This registration is currently achieved using one of two methods. One method involves the use of a stereotactic headframe, a rigid box-like device that is affixed to the patient's skull. The headframe defines a known volume of space that can be described by a specific coordinate system. The other method involves the use of rigid bone fiducials temporarily implanted in the patient's skull. These bone fiducials create landmarks that are used in the same way that the rigid portions of the headframe are used to define a known volume in space. Once the headframe or bone fiducials are in place, a series of stereotactic radiographic examinations are obtained, which typically include CT and/or MRI. The images obtained have the patient's specific brain anatomy as well as the landmarks (headframe or bone fiducials) defining the known volume and coordinate system. These images are fed into a commercially available stereotactic computer system to form a targeting database. The intended target is manually identified on the computer images by a clinician, and then the computer uses this information to determine and display the spatial coordinates of the selected target. In headframe-based systems, a coordinate and trajectory, which can be mechanically set on the headframe, are provided by the computer so that the target lies at a particular depth on the path of the insertion arm. In the bone-fiducials based systems, an electromagnetic or optical tracking system guides the targeting based on real-time feedback of the positions of the implanted bone markers and the position and orientation of the insertion arm.

After the construction of the three-dimensional stereotactic targeting dataset, the implantation of the various components of a neuromodulation system of the present disclosure is accomplished, which is similar to standard stereotactic surgical procedures used to implant DBS electrodes. In an embodiment, a burrhole is made in the patient's calvarium or dome of the skull. Next, using the stereotactic guidance system describe above and the trajectory determined by pre-operative imaging, the target is mapped by inserting microelectrodes and/or semi-microelectrodes in a serial and/or parallel manner. These electrodes are capable of recording electrical signals specific to the intended target, as well as other brain structures along the trajectory. In an embodiment, for use in Parkinson's disease, the target may include the substantia nigra (SN), which has a well-defined electro-neurophysiological signal. Once the location of the target is confirmed by this mapping process, the light delivery components of the system are implanted. As appropriate for the particular system being implanted, one or more components may be affixed to the skull by any several methods similar to those used in DBS surgery, and connections may be made to various central housings and light housings.

Alternatively, this implantation procedure could be done without physiological mapping in an interventional MRI suite. In this manner, the system components would be implanted in the same fashion, but the target would be confirmed by direct visualization using MRI sequences designed specifically to image the intended target.

Particular procedures used to implant components to irradiate cortical targets are similar to the procedures currently used to implant chronic surface electrodes, which can be implanted epidurally or subdurally. These procedures are fairly standardized, but multiple nuances exist, depending on the preferences of the particular surgeon and the particular equipment used. Such electrical stimulation-based neuromodulation systems are currently used as treatment for refractory pain, stroke recovery, and tinnitus.

First, imaging similar to that described above for DBS implantation is obtained. In addition to these images, certain functional imaging sequences such as PET, MRI, and MEG may further characterize the intended target and obviate the need for physiological confirmation with mapping.

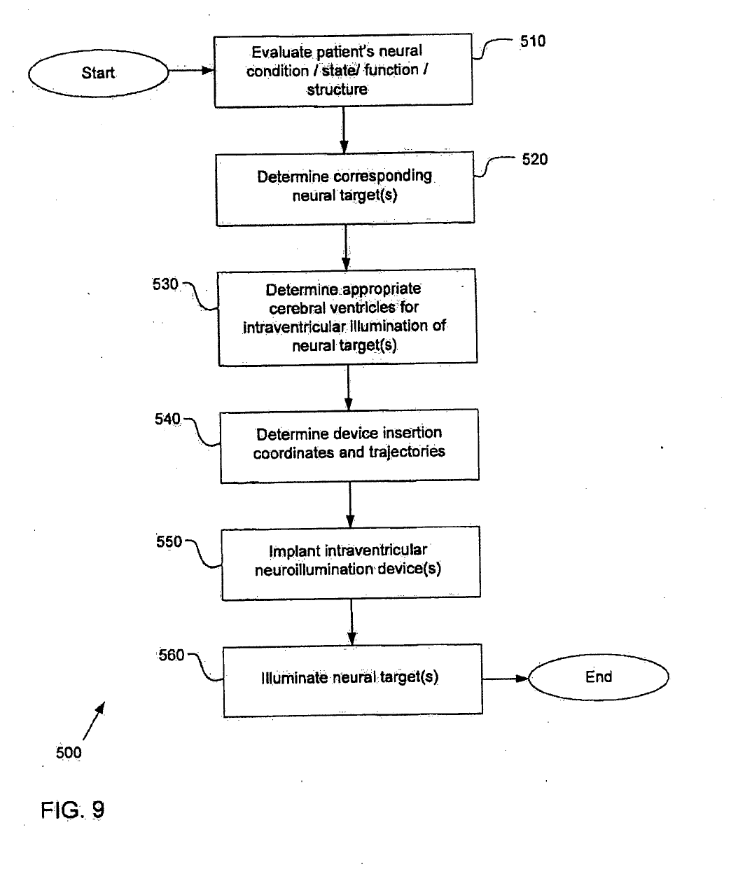

When the components to be implanted are relatively large in surface area compared to the size of the burrhole, such as the illumination paddles 204a, 204b, and 204c respectively shown in FIGS. 9A, 9B, and 9C, a craniotomy may be performed using optical and/or EM stereotactic guidance. The location of cortical targets may be defined by physiological mapping methods such as electrocorticography (ECoG). The components to irradiate a cortical target will be placed over the surface of the target. In the case of subdural implantation, the dura will be opened enough to allow the components to be placed safely. Components implanted epidurally or subdurally will be affixed to the dura using non-absorbable sutures. As appropriate for the particular system being implanted, one or more components may be affixed to the skull by any several methods similar to those used in DBS surgery, and connections may be made to various central housings and light housings.

These methods apply to the implantation of components to irradiate cortical targets, which can include various types of IDUs and/or optical fiber bundles as described and/or shown herein.

In some embodiments, the central housing device is suitably implanted in the chest using methods similar to those currently employed to implant a pulse generator housing in DBS systems. Depending upon embodiment details in accordance with the present disclosure, wires and/or optical fiber bundles can be tunneled subdermally from the patient's head to the central housing, and then appropriate couplings or connections can be made based upon the particular embodiment under consideration.

It is contemplated that various implantation and illumination techniques, procedures, or methods based upon an appropriate embodiment of a BNS in accordance with the present disclosure can be used to treat many and varied neurological disorders or diseases.

Neural Targets Treatment Protocols, and/or Procedures

The systems and methods of the present disclosure can be used to treat many and varied neurological disorders or diseases. Such use is exemplified for the following diseases.

In an embodiment, a brain neuromodulation system of the present disclosure can be used for the treatment of Parkinson's Disease (PD). Targets are suitably the substantia nigra pars compacta and reticularis, and posterior putamen. The pathognomonic event in PD is cell death in the cell population of the substantia nigra pars compacta. The loss of dopamine produced by these cells results in nearly all of the manifestations of PD. The common pathophysiological mechanism that ultimately results in cell death in PD is due to oxidative stress especially at the level of the mitochondria. Reduced metabolic activity and mechanisms of apoptotic cell death have also been implicated. The brain neuromodulation systems of the present disclosure are capable of reducing oxidative stress/apoptotic proteins, as well as, increasing/supporting anti-apoptotic proteins and mitochondrial metabolism. This will result in a reduction and/or halting of the cell death in the substantia nigra pars compacta (and its projections to the motor/posterior portions of the putamen) in PD. This in turn will maintain a normal dopaminergic concentration in the brain and result in improved motor and non-motor function.

In an embodiment, a brain neuromodulation system of the present disclosure can be used for the treatment of Alzheimer's Disease (AD). Targets are suitably hippocampus and septal region/nucleus basalis of Meynert. The pathognomonic event in AD is the development of beta-amyloid protein deposits with subsequent cell-death. The primary pathophysiological mechanisms that ultimately results in these phenomena in AD is oxidative stress especially at the level of the mitochondria. Mechanisms of apoptotic cell death have also been implicated. Current treatment strategies in AD treatment involve drugs and dietary interventions designed to address these interactions. Two areas of the brain that undergo these changes are the hippocampus and the septal regions of the medial forebrain. The pathological mechanisms that occur in these regions account for much of the disability associated with AD. The brain neuromodulation systems of the presently disclosed embodiments can reduce oxidative stress/apoptotic proteins, as well as, increase/support anti-apoptotic proteins and mitochondrial metabolism. This will result in the reduction and/or halting of the cell death in the hippocampus/septal regions associated with AD. This in turn will maintain a normal neuronal population in the brain and result in improved cognitive and memory function.

In an embodiment, a brain neuromodulation system of the present disclosure can be used for the treatment of brain injury and stroke. Targets are various cortical and subcortical regions depending on the location of the event. Stroke and traumatic brain injury results in altered oxygen metabolism in the particular brain region affected. These changes result in increased levels of oxidative stress with subsequent cell death in regions that are not killed outright by the initial insulting event. The brain neuromodulation systems of the presently disclosed embodiments can deliver light into the region of the brain that has recently experienced the traumatic injury/stroke, and can preserve “at-risk” neuronal pools and maximize recovery. These “at-risk” neuronal pools will be helped by the light's ability to augment cytochrome activity, reduce the effects of oxidative stress, and increase the local nitric oxide concentration with subsequent augmentation of blood flow to penumbra regions.

In an embodiment, a brain neuromodulation system of the present disclosure can be used for the treatment of Huntington's Disease (HD). Targets are suitably the putamen and the caudate nucleus of the striatum. The pathognomonic event in HD is the development of a specific genetic mutation with subsequent selective cell-death. The common pathophysiological mechanism that ultimately results in these phenomena in HD is due to oxidative stress especially at the level of the mitochondria. Mechanisms of apoptotic cell death have also been implicated. Current strategies in HD treatment involve drugs and dietary interventions designed to address these interactions. Two areas of the brain that undergo the majority of these changes are the putamen and the caudate nucleus of the striatum. The pathological mechanisms that occur in these regions (with their projections to motor and non-motor areas of the cerebral cortex) account for much of the disability associated with HD. The brain neuromodulation systems of the present disclosure can both reduce oxidative stress/apoptotic proteins and increase/support anti-apoptotic proteins and mitochondrial metabolism. This will result in the reduction and/or halting of the cell death in the striatal regions in HD. This in turn will maintain a normal neuronal population in the brain and result in improved cognitive and motor function.

### Intraventricular Implantation and Transventricular Neuroillumination Processes

FIG. 9 is a flow diagram of a representative embodiment of an intraventricular implantation process 500 according to the present disclosure. The process 500 includes evaluating a patient's neurological condition, symptomatic state, neural structure (e.g., using MRI), and/or neural function or dysfunction (e.g., using fMRI or EEG) in a first process portion 510. A second process portion 520 can include identifying or determining a set of neural targets expected to be responsible for controlling, contributing to, or affecting particular aspects of the patient's condition, symptomatic state, or neurologic dysfunction. A third process portion 530 can include determining one or more appropriate cerebral ventricles into which a set of intraventricular/transventricular illumination devices can be implanted to apply, deliver, emanate, or propagate optical signals to, into, or through at least some of the neural targets identified in process portion 520. An appropriate set of intraventricular device insertion locations and corresponding neuroanatomical or stereotactic coordinates and device insertion trajectories is determined in a fourth process portion 540, and one or more intraventricular or transventricular neuroillumination devices. A fifth process portion 550 can include implanting one or more intraventricular neuroillumination systems or devices, such as those previously described herein. Finally, a sixth process portion 560 can include applying optical signals to neural targets at one or more times (e.g., in accordance with a set of program instructions, and/or in response to sensed information) using the implanted intraventricular neuroillumination devices. Light emitted, emanated, or generated by an intraventricular neuroillumination device can propagate transventricularly (through a neural tissue/ventricle boundary) to one or more particular neural targets.

### Neuroillumination Plus Electrical Stimulation Processes

Electrical stimulation can induce neuronal damage within a neural target when the electrical stimulation is applied or delivered in a manner that exceeds known electrical charge injection or charge density limits. Foundational research relating to manners and/or situations in which electrical stimulation can induce neuronal injury or damage has been performed by Douglas McCreery, and is described in “Charge Density and Charge per Phase as Cofactors in Neuronal Injury Induced by Electrical Stimulation,” IEEE Transactions on Biomedical Engineering, Bol. 37, No. 10, October 1990, pp. 996-1001; and “The Effects of Prolonged Intracortical Microstimulation on the Excitability of Pyramidal Tract Neurons in the Cat,” Annals of Biomedical Engineering, Vol. 30, pp. 107-119, each of which is incorporated herein by reference in its entirety. An established or generally recognized safe upper charge per phase or charge density limit for electrical stimulation is known as a McCreery limit.

A given electrical stimulation pulse can include one or more pulse phases that deliver electrical charge to neural tissue. Charge density is defined as charge per pulse phase divided by the surface area of the electrodes that are actively involved in charge delivery. In general, conservative or expected safe charge density limits can depend upon electrode composition (e.g., Platinum, Platinum-Iridium, or Iridium Oxide) and a manner in or location to which electrical stimulation is applied (e.g., at a subcortical deep brain target, at a subdural cortical (e.g., extracerebral) location, or at an epidural cortical location). For Platinum electrodes, in certain electrical stimulation situations, a charge density limit can be approximately 20, 30, 50, or possibly 100 Microcoulombs/cm². A conservative or generally conservative charge density limit is 30 Microcoulombs/cm² for a standard DBS electrode exhibiting an impedance of approximately 500 Ohms. A reference upper electrical stimulation limit can correspond to a given charge per phase or a charge density limit.

In an embodiment of the present disclosure where both neuroillumination and electrical stimulation are applied to a neural target, neuroillumination can be delivered to the neural target before, during, and/or after the neural target is electrically stimulated. Such neuroillumination can induce or result in a neuroprotective effect within targeted neurons that are exposed to prolonged, excessive, or overly intense electrical stimulation. Moreover, in certain embodiments, a neuroprotective effect provided by or resulting from such neuroillumination can facilitate the electrical stimulation of a neural target at a charge per phase or a charge density that exceeds or is higher than a conventional or reference upper electrical stimulation limit in the absence of neuroillumination. In an embodiment, neuroillumination can be provided in a manner that is responsive to the application of electrical signals to a neural target relative to one or more predetermined or programmable upper electrical stimulation limit.

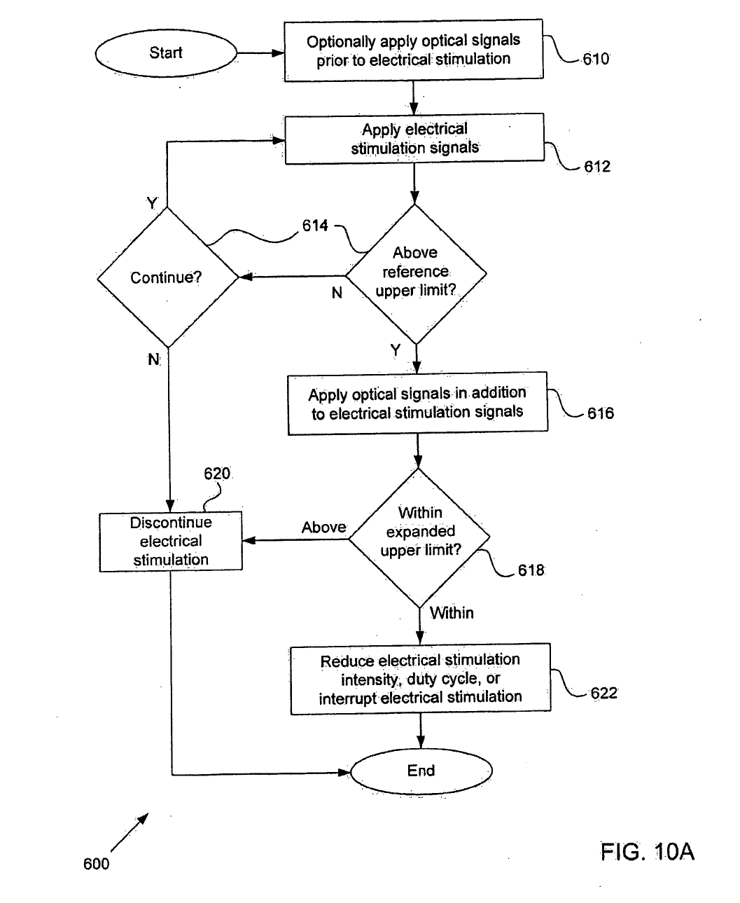

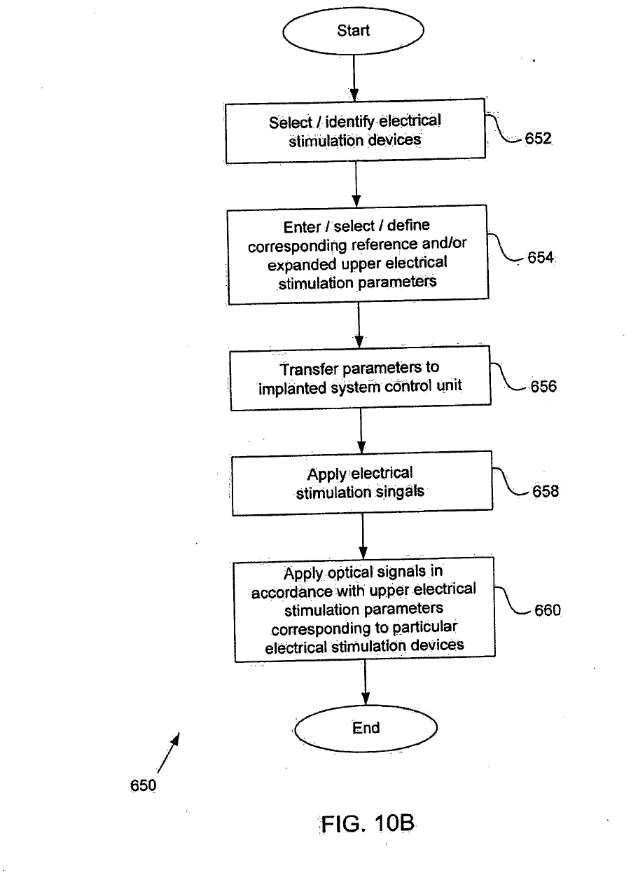

FIG. 10A is a flow diagram of a representative optical illumination and electrical stimulation process 600 according to an embodiment of the present disclosure. In a first process portion 610, neuroillumination is optionally applied (e.g., in one or more previously described manners) to a neural target prior to the application of electrical stimulation signals to the neural target. In a second process portion 612, electrical stimulation is applied to a neural target, typically in accordance with parameters specified in accordance with a set of stored program instructions. A third process portion 614 can include determining whether the electrical stimulation is applied near, at, or above a reference upper electrical stimulation limit, level, or intensity (e.g., an expected safe charge per phase or charge density level). Depending upon embodiment details, a reference upper electrical stimulation limit can be or correspond to a predetermined charge per pulse phase or a given charge density. If the electrical stimulation is applied below a reference upper electrical stimulation limit, the third process portion 614 can include determining whether to continue or terminate electrical stimulation.

If the electrical stimulation is applied at, near, or above a reference upper electrical stimulation limit, neuroillumination can be applied to the neural target concurrent with the electrical stimulation of the neural target in a fourth process portion 616. Such neuroillumination can be applied or delivered in one or more manners, for example, at a wavelength of approximately 670 and/or 820 nm, for approximately 2-20 minutes (e.g., approximately 6-12 minutes), and a power level of approximately 6-14 mW/cm² or approximately 10 mW/cm².

A fifth process portion 618 can include determining whether the electrical stimulation delivered to the neural target is applied below, near, approximately at, or beyond an expanded upper electrical stimulation limit that can correspond to an expected level or extent of neuroprotective benefit that the neural target can experience as a result of the optical signals applied in association with the fourth process portion 616. Depending upon embodiment details, an expanded upper electrical stimulation limit can be defined as a charge per phase, charge density, or stimulation parameter level or intensity (e.g., current density or current amplitude, and/or cumulative or continuous electrical stimulation time) that exceeds the reference upper electrical stimulation limit by a minimum or given percentage (e.g., approximately 5%-20%, or approximately 10%-15%).

If the electrical stimulation is applied beyond or above an expanded upper electrical stimulation limit, a sixth process portion 620 can include discontinuing the electrical stimulation. If the applied electrical stimulation falls within a range that is above a reference upper electrical stimulation limit and below an expanded upper electrical stimulation limit, a seventh process portion 622 can include reducing the intensity (e.g., by reducing a peak current or voltage amplitude and/or a pulse repetition frequency by a given amount or percentage, such as 5%-20%, or 10%-15%) of the electrical stimulation; or duty cycling the electrical stimulation; or interrupting or discontinuing the electrical stimulation either immediately, or following an acceptable or allowable above limit time period or interval. A representative above limit time interval can be, for example, approximately 1-30 minutes (e.g., 10-20 minutes, or 15 minutes).

In certain embodiments, duty cycled electrical stimulation can involve the repeated or cyclical application of electrical stimulation during an ON interval, followed by a cessation of electrical stimulation during an OFF interval. Representative ON and OFF intervals can be, for example, a given number of seconds (e.g., 5-60 seconds) or minutes (e.g., 1-5 minutes).

An interruption of electrical stimulation can occur for a predetermined interruption period, for example, between 15 minutes and 1 or more hours (e.g., 2 hours), possibly depending upon an effect the interruption period is expected to have upon one or more patient symptoms. Following an interruption period, electrical stimulation can be resumed, typically at a level below the reference upper electrical stimulation limit.

A given BNS of the present disclosure can include one or more electrical stimulation devices, which can be identical or different in structure and/or function (e.g., a set of electrical stimulation devices can include a DBS electrode and one or more cortical stimulation electrode). Reference upper electrical stimulation limits or parameters, and/or expanded upper electrical stimulation limits or parameters, can differ from one type of electrical stimulation device to another.

FIG. 10B is a flow diagram of a representative process 650 for specifying or selecting a set of reference upper electrical stimulation parameters and/or expanded upper electrical stimulation parameters corresponding to at least one electrical stimulation device that will apply electrical stimulation in association with a treatment program under consideration. A first process portion 652 can involve physician or clinician selection or identification of one or more electrical stimulation devices included in a BNS of the present disclosure through the use of a Graphical User Interface (GUI) that executes on the external programming and diagnostic system 90. A second process portion 654 can include the entry, definition, selection, or storage of reference and/or expanded upper electrical stimulation parameters corresponding to such electrical stimulation devices.

A third process portion 656 can include the transfer of reference and/or expanded upper electrical stimulation parameters corresponding to particular electrical stimulation devices from the external programming and diagnostic system 90 to the memory 126 within the system control unit 120 of the BNS. A fourth process portion 658 can include applying electrical stimulation signals to particular neural targets using the electrical stimulation devices associated with the treatment program under consideration; and a fifth process portion 660 can include applying optical illumination signals a given neural target receiving electrical stimulation based upon a reference and/or an expanded upper electrical stimulation limit corresponding to the particular electrical stimulation device that is applying electrical stimulation signals to this neural target.

Various portions of processes relating to optical illumination and electrical stimulation, such as processes 600, 650 described above, can be performed automatically or semi-automatically in accordance with a set of program instructions. Such program instructions can be stored in a computer readable medium such as a memory, and executed by a processor that resides internal or external to a patient as appropriate.

### Representative Sensing Based and NIRS-Related Processes

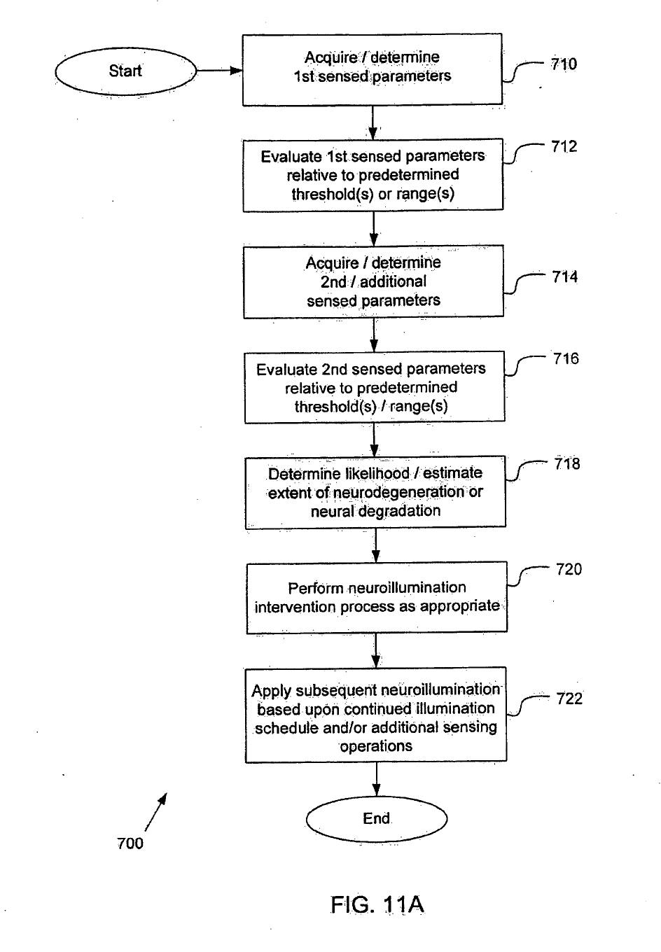

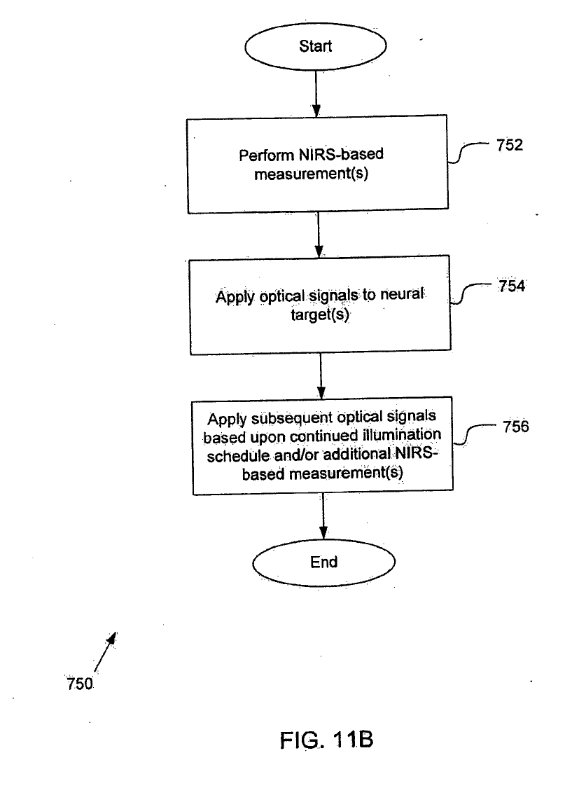

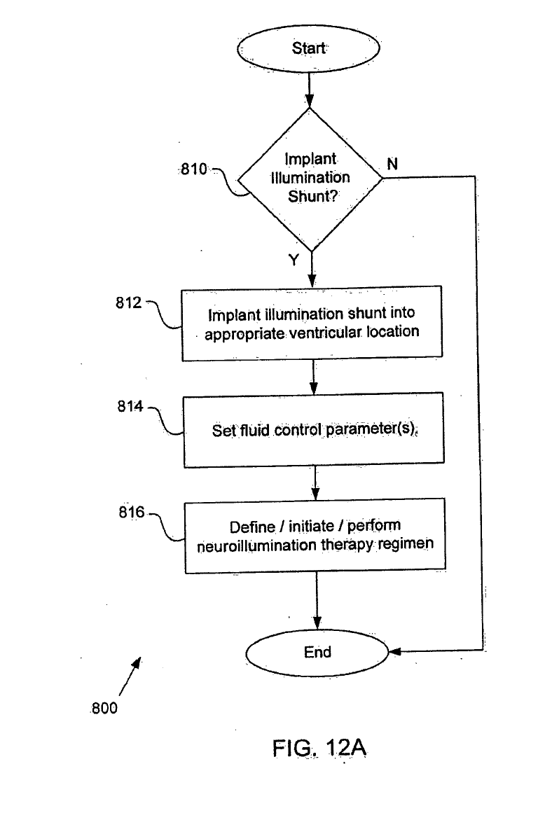

FIG. 11A is a flow diagram of a representative sensing based neuroillumination process 700 according to an embodiment of the disclosure. The process 700 can include a first process portion 710 that can include acquiring, calculating, generating, determining, or retrieving a first set of sensed parameters. A second process portion 712 can include analyzing, characterizing, or evaluating the first set of sensed parameters relative to a predetermined sensed parameter range, threshold, or limit. An optional third process portion 714 can include acquiring a second or additional set of sensed parameters, possibly based upon an analysis of the first set of sensed parameters in association with the second process portion 712. Depending upon embodiment details, the particular physiologic characteristic(s) to which the first set of sensed parameters corresponds can be identical to or different than the physiologic characteristic(s) to which the second or additional set of sensed parameters corresponds. For example, the first set of sensed parameters can include or correspond to one or more NIRS measurements, and the second set of sensed parameters can exclude NIRS measurements but include or correspond to one or more temperature or pH measurements. If second or additional sensed parameters are acquired, such sensed parameters can be acquiring or determining a second set of sensed parameters. In the event that a second or additional set of sensed parameters is acquired, a fourth process portion 716 can include analyzing such sensed parameters relative to a sensed parameter range, threshold, or limit.

A fifth process portion 718 can include determining whether neurodegeneration or neural degradation has occurred, is occurring, or is likely or expected to occur; and/or estimating a level or extent of neurodegeneration. If neurodegeneration is possible, likely, or expected, a sixth process portion 720 can include initiating or performing a neuroillumination intervention process that involves the application of light to one or more neural targets, such as neural targets adjacent or proximate to particular sensing devices that acquired or transferred sensed parameters in association with the first and/or third process portion 710, 714. Finally, a sixth process portion 720 can include applying additional or subsequent neuroillumination to particular neural targets, possibly based upon additional sensing operations or in accordance with a continued neuroillumination schedule (e.g., twice per day, for a period of three months or on a chronic basis).

FIG. 11B is a flow diagram of a representative NIRS-related optical illumination process 750 according to an embodiment of the present disclosure. The process 750 can include a first process portion 752 that includes performing at least one NIRS-based measurement to determine or estimate one or more of a cytochrome oxidase condition or redox state, an oxyhemoglobin state, or a deoxyhemoglobin state. In certain embodiments, a NIRS-based measurement can involve energizing a set of near infrared light sources that apply optical signals to a neural target; detecting backscattered or reflected light with one or more photodetectors to determine current near infrared absorption characteristics corresponding to the neural target; and calculating or correlating such absorption characteristics with a cytochrome oxidase redox state, an oxyhemoglobin state, and/or a deoxyhemoglobin state.

A second process portion 754 can include the illumination or optical irradiation of the neural target in one or more manners previously described if the NIRS-based measurement(s) indicate an undesirable, unacceptable, high, or excessive level of oxidative stress or metabolic dysfunction exists within the neural target. A third process portion 756 can include the subsequent illumination of the neural target at particular times, such as in accordance with a predetermined neuroillumination schedule (e.g., one, two, or three times per day). The neuroillumination schedule can remain in effect on a continued or repeated basis as long as a NIRS-based measurement indicates the presence or occurrence of an undesirable level of oxidative stress or metabolic dysfunction, for example, across a predetermined number of NIRS-based measurements (e.g., five or more NIRS-based measurements out of eight measurements total) relative to a given time period (e.g., two days).

Various sensing operations or portions of sensing based processes (e.g., the processes 700, 750 described above) associated with providing neuroillumination in association with sensing operations in association with relating to optical illumination and electrical stimulation can be performed automatically or semi-automatically in accordance with a set of program instructions. Such program instructions can be stored in a computer readable medium such as a memory, and executed by a processor within the BNS.

### Representative Illumination Shunt Processes

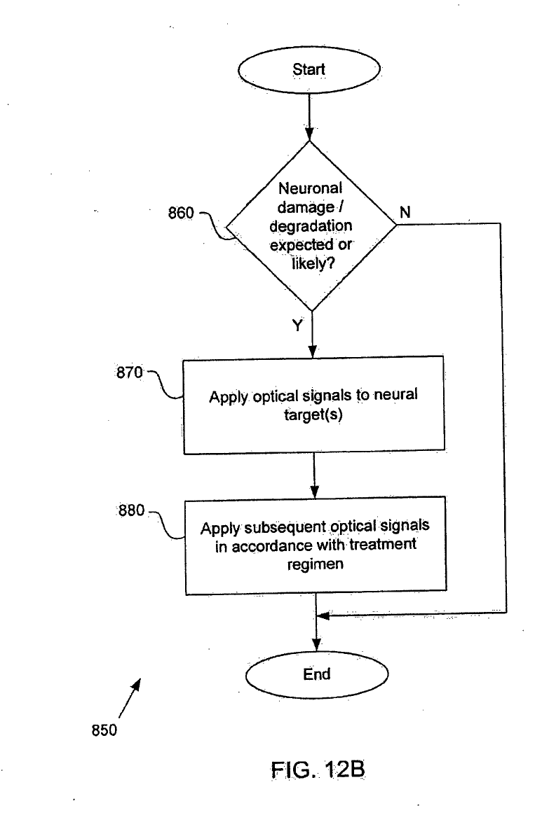

FIG. 12A is a flow diagram of a representative illumination shunt implantation and operation process 800 according to an embodiment of the present disclosure. The process 800 can include a first process portion 810 that can include determining whether a patient requires or would benefit from the implantation of an illumination shunt. In a situation in which the implantation of a shunt device is required or warranted, selection of an illumination shunt (versus a conventional shunt) can be based upon a possibility, likelihood, or expectation that neuroillumination can limit or prevent neurodegeneration or neuronal damage. The first process portion 810 could involve, for example, a surgeon's decision that an intraventricular hemorrhage intervention for a prematurely born baby is required or warranted, and that neuroillumination can limit a manifestation of neurologic dysfunction (e.g., associated with cerebral palsy). If a patient would benefit from the implantation of an illumination shunt, a second process portion 812 can include the surgical implantation of a portion of an illumination shunt into an appropriate ventricular location, where the illumination shunt has a shape or geometry that is appropriate for the size, condition, and anatomy of the patient under consideration. The implantation of a illumination shunt (e.g., of a type that is generally identical or analogous to that described above with reference to FIG. 4A), and the application of neuroillumination to neural targets using such a shunt, can provide or result in a neuroprotective benefit within the neural targets, increase a likelihood that “at risk” neurons (e.g., neurons at risk of significant cellular damage, or cell death) will survive, and possibly prevent generally unaffected or healthy neurons from becoming “at risk” neurons (e.g., as a result of subsequent slow or Wallerian type degeneration that can be due to disruption of normal neuronal communication processes).

A third process portion 814 can include defining, establishing, setting, or adjusting a fluid control parameter (e.g., a fluid pressure threshold) for the illumination shunt. In an embodiment, an external programming and diagnostic system can wirelessly communicate the fluid control parameter to a shunt control module in response to physician input (e.g., as facilitated by a GUI) received or accepted by the external programming and diagnostic system. A fourth process portion 816 can include defining, initiating, and/or performing a neuroillumination therapy regimen or program, which can activate or operate a set of light sources to illuminate neural targets (e.g., by way of intraventricular and/or extraventricular neuroillumination, which can be applied in a manner that is identical or analogous to that described for particular embodiments of the present disclosure). The fourth process portion 816 can involve communication between an external programming and diagnostic system and a BNS control module, for instance, in response to physician input received by the external programming and diagnostic system. The fourth process portion 816 can further include initiating neuroillumination operations in response to receipt of input from a patient or a patient caretaker, where such input can be entered or received by an external patient controller.

FIG. 12B is a flow diagram of a representative illumination shunt process 850 according to an embodiment of the present disclosure. The process 850 can include determining whether a sensed parameter indicates that neuronal damage is expected or likely to occur or has occurred in a first process portion 860. The sensed parameter can be, for example, a fluid pressure and/or the presence or level of a chemical substance or molecular structure. If neuronal damage is expected to occur or has occurred, a second process portion 870 can include activating or energizing the set of light sources 142, and applying or delivering optical signals to one or more neural targets, for example, in one or more manners previously described. A third process portion 880 can include applying subsequent optical signals to particular neural targets at particular times, possibly in accordance with a treatment or illumination regimen that can involve the application of optical signals to neural targets one, two, or three times per day as long as a sensed parameter indicates that neuronal damage remains or is likely or possible; and/or for a given number of days or weeks after a sensed parameter indicates that a likelihood of neuronal damage is low or normal (e.g., after a sensed parameter returns or transitions to a normal or acceptable level).

Various operations or portions of illumination shunt operation processes, including portions of a sensing based illumination shunt process (e.g., the processes 850 described above) can be performed automatically or semi-automatically in accordance with a set of program instructions. Such program instructions can be stored in a computer readable medium such as a memory, and executed by a processor within the BNS.

The processing performed by the system described herein may be performed by a general purpose computer alone or in connection with a specialized processing computer. Such processing may be performed by a single platform or by a distributed processing platform. In addition, such processing and functionality can be implemented in the form of special purpose hardware or in the form of software being run by a general purpose computer. Any data handled in such processing or created as a result of such processing can be stored in any memory as is conventional in the art. By way of example, such data may be stored in a temporary memory, such as in the RAM of a given computer system or subsystem. In addition, or in the alternative, such data may be stored in longer-term storage devices, for example, magnetic disks, rewritable optical disks, and so on. For purposes of the disclosure herein, a computer-readable media may comprise any form of data storage mechanism, including such existing memory technologies as well as hardware or circuit representations of such structures and of such data.

A neuromodulation method includes applying electrical signals to a neural target; determining if the electrical signals are applied in a manner that exceeds a predetermined or programmable reference upper electrical stimulation limit; and applying optical signals to at least a portion of the neural target. In an embodiment, the method further comprises determining if the electrical signals are applied in a manner that exceeds an expanded upper electrical stimulation limit. In an embodiment, the method further comprises discontinuing the application of electrical signals in the event that the electrical signals are applied in a manner that exceeds the expanded upper electrical stimulation limit. In an embodiment, the method further includes determining if the electrical signals are applied in a manner that exceeds the reference upper electrical stimulation limit and which is below the expanded upper electrical stimulation limit; and continuing to apply electrical signals to the neural target.

A neuroillumination method includes the steps of (a) acquiring a first set of sensed parameters corresponding to a neural target using a first set of sensing devices; (b) acquiring a second set of sensed parameters corresponding to the neural target using a second set of sensing devices; (c) determining whether at least one set of sensed parameters corresponds to at least one from the group of an oxidative stress condition, a neurodegeneration condition, a neuronal damage condition, and an abnormal metabolic condition within the neural target; and (d) applying optical signals to the neural target. In an embodiment, the method further includes determining whether the first set of sensed parameters and the second set of sensed parameters correspond to at least one from the group of an oxidative stress condition, a neurodegeneration condition, a neuronal damage condition and an abnormal metabolic condition within the neural target.

A neuroillumination method includes measuring a bodily fluid pressure parameter; determining whether the bodily fluid pressure parameter exceeds a reference fluid pressure value; and applying optical signals to a neural target.

A method for treating a neural condition of a patient includes implanting an illumination probe into a cerebral ventricle of the patient, the illumination probe operable to emit near-infrared light; and preferentially directing near-infrared light emitted by the illumination probe toward a predetermined set of neural targets.

A tissue illumination method includes implanting an illumination shunt system into a patient, the illumination shunt system including: a ventricular shunt apparatus; a tissue illumination apparatus; and a telemetry circuit coupled to at least one of the ventricular shunt apparatus and the tissue illumination apparatus; establishing a wireless communication link between the telemetry circuit and a programming device; and wirelessly communicating one from the group of a shunt control parameter and a tissue illumination parameter from the programming device to the telemetry circuit. In an embodiment, the method further includes illuminating neural tissue with near-infrared light. In an embodiment, the method further includes preferentially applying illumination to a predetermined set of neural targets, the illumination applied at a wavelength ranging from about 670 nm to about 820 nm.

All patents, patent applications, and published references cited herein are hereby incorporated by reference in their entirety. It will be appreciated that several of the above-disclosed and other features and functions, or alternatives thereof, may be desirably combined into many other difference systems or applications. Various presently unforeseen or unanticipated alternatives, modifications, variations, or improvements therein may be subsequently made by those skilled in the art which are also intended to be encompassed by the following claims.

What is claimed is:

1. A neuroillumination probe comprising:
an elongate member;
a first light emission site carried by the elongate member; and
a second light emission site carried by the elongate member,
wherein the first light emission site and the second light emission site are separated along a length of the elongate member by an expected approximate distance between a deep brain neural target and a cortical neural target.

2. The neuroillumination probe of claim 1, wherein the deep brain neural target comprises cortical neural tissue.

3. A neuroillumination probe comprising:
an elongate member including a proximal segment and a distal segment, the proximal segment having a different geometric shape than the distal segment; and
a first light emission site carried by the elongate member,
wherein the distal segment has a geometric shape that corresponds to a boundary of an anatomical structure.

4. The neuroillumination probe of claim 3, wherein the anatomical structure comprises a portion of a cerebral ventricle.

5. The neuroillumination probe of claim 3, wherein the anatomical structure comprises a portion of one from the group of the lateral cerebral ventricle and the third cerebral ventricle.

6. The neuroillumination probe of claim 3, wherein the first light emission site carries or is optically coupled to a near-infrared light source.

7. The neuroillumination probe of claim 3 further comprising a second light emission site carried by the elongate member.

8. The neuroillumination probe of claim 7 wherein the first light emission site is carried by the proximal segment of the elongate member and the second light emission site is carried by the distal segment of the elongate member.

9. A brain neuroillumination system comprising:
an implantable housing;
a control unit carried by the implantable housing;
a neuroillumination probe including an elongate portion and a contoured portion, the neuroillumination probe having at least a first light emission site; and
a light source operatively coupled to the control unit and optically coupled to the first light emission site, the light source carried by one from the group of the implantable housing and the neuroillumination probe,
wherein the contoured portion of the neuroillumination probe has a geometric shape that corresponds to a boundary of an anatomical structure.

10. The brain neuroillumination system of claim 9 wherein the light source comprises a near-infrared light source.

11. The brain neuroillumination system of claim 9 wherein the light source outputs near-infrared light at a wavelength ranging from about 670 nm to about 820 nm.

12. The brain neuroillumination system of claim 9 wherein the anatomical structure comprises a portion of a cerebral ventricle.

13. The brain neuroillumination system of claim 9 wherein the anatomical structure comprises a portion of one from the group of the lateral cerebral ventricle and the third cerebral ventricle.

14. The brain neuroillumination system of claim 9 wherein the first light emission site is carried by the contoured portion of the neuroillumination probe.

15. The brain neuroillumination system of claim 14 wherein the neuroillumination probe further comprises a second light emission site that is carried by the contoured portion of the neuroillumination probe.

16. The brain neuroillumination system of claim 14 wherein the neuroillumination probe further comprises a second light emission site that is carried by the elongate portion of the neuroillumination probe.

17. The brain neuroillumination system of claim 9 further comprising:
a telemetry circuit carried by the implantable housing; and
a programming device configured for wireless signal communication with the telemetry unit.

18. A brain neuroillumination system comprising:
an implantable housing;
a control unit carried by the implantable housing;
a neuroillumination delivery device including at least one light emission site;
at least one light source operatively coupled to the control unit and optically coupled to the at least one light emission site; and
a near-infrared spectroscopy unit coupled to the control unit,
wherein the near-infrared spectroscopy unit is configured to detect an optical signal corresponding to at least one from the group of a cytochrome oxidase state and a hemoglobin oxygenation state.

19. An illumination shunt system comprising:
a shunt catheter;
a distal catheter;
a fluid reservoir in fluidic communication with the shunt catheter and the distal catheter;
a shunt control module;
a one-way valve operatively coupled to the shunt control module and in fluidic communication with the shunt catheter;
a light source;
an implantable illumination probe including at least one light emission site that is optically coupled to the light source; and
an illumination control module operatively coupled to the light source.

20. The illumination shunt system of claim 19 further comprising an implantable housing that carries the shunt control module and the illumination control module.

21. The illumination shunt system of claim 19 wherein the light source comprises a near-infrared light source.

22. The illumination shunt system of claim 19 wherein the light source outputs near-infrared light at a wavelength ranging from about 670 nm to about 820 nm.

23. The illumination shunt system of claim 19 wherein a portion of the implantable illumination probe is carried by the shunt catheter.

24. The illumination shunt system of claim 19 wherein a portion of the implantable illumination probe is carried within a portion of the shunt catheter.

25. The illumination shunt system of claim 19 wherein a portion of the implantable illumination probe is seated in a portion of the shunt catheter.

26. The illumination shunt system of claim 20 wherein the fluid reservoir is carried by the implantable housing.

27. The illumination shunt system of claim 20 wherein the light source is carried by one from the group of the implantable housing and the implantable illumination probe.

28. The illumination shunt system of claim 19 further comprising:
a telemetry circuit coupled to the illumination control module; and
a programming device configured for wireless communication with the telemetry circuit.

29. The illumination shunt system of claim 28 wherein the telemetry circuit is further coupled to the shunt control module.

30. A neuromodulation method comprising:
applying electrical signals to a neural target;
determining if the electrical signals are applied in a manner that exceeds a predetermined or programmable reference upper electrical stimulation limit; and
applying optical signals to at least a portion of the neural target.

31. The method of claim 30 wherein determining if the electrical signals are applied in a manner that exceeds the reference upper electrical stimulation limit is performed automatically.

32. The method of claim 30 wherein the steps of determining if the electrical signals are applied in a manner that exceeds the reference upper electrical stimulation limit and applying optical signals to the neural target are performed automatically.

33. The method of claim 30 wherein the reference upper electrical stimulation limit corresponds to a charge density.

34. The method of claim 30 wherein the optical signals are applied in the event that the electrical signals are applied in a manner that exceeds the reference upper electrical stimulation limit.

35. The method of claim 30 further comprising determining if the electrical signals are applied in a manner that exceeds an expanded upper electrical stimulation limit.

36. The method of claim 35 wherein the expanded upper electrical stimulation limit is greater than the reference upper electrical stimulation limit by a predetermined percentage.

37. The method of claim 35 wherein the expanded upper electrical stimulation limit is greater than the reference upper electrical stimulation limit by approximately 5% to approximately 20%.

38. The method of claim 35 wherein the expanded upper electrical stimulation limit is greater than the reference upper electrical stimulation limit by approximately 10% to approximately 15%.

39. The method of claim 35 wherein the expanded upper electrical stimulation limit is greater than the reference upper electrical stimulation limit by at least approximately 10%.

40. The method of claim 35 further comprising discontinuing the application of electrical signals in the event that the electrical signals are applied in a manner that exceeds the expanded upper electrical stimulation limit.

41. The method of claim 40 wherein discontinuing the application of electrical signals is performed automatically.

42. The method of claim 35 further comprising:
determining if the electrical signals are applied in a manner that exceeds the reference upper electrical stimulation limit and which is below the expanded upper electrical stimulation limit; and
continuing to apply electrical signals to the neural target.

43. The method of claim 42 wherein continuing to apply electrical signals to the neural target comprises applying one from the group of a reduced amplitude electrical signal, applying a reduced frequency electrical signal, and applying a duty cycled electrical signal to the neural target.

44. The method of claim 42 wherein continuing to apply electrical signals to the neural target comprises:
interrupting the application of electrical signals to the neural target for a predetermined time interval; and
resuming the application of electrical signals to the neural target after the predetermined time interval.

45. The method of claim 42 wherein continuing to apply electrical signals to the neural target comprises:
applying electrical signals to the neural target for a predetermined time interval; and
discontinuing the application of electrical signals to the neural target after the predetermined time interval.

46. A neuroillumination method comprising the steps of:
(a) acquiring a first set of sensed parameters corresponding to a neural target using a first set of sensing devices;
(b) acquiring a second set of sensed parameters corresponding to the neural target using a second set of sensing devices;
(c) determining whether at least one set of sensed parameters corresponds to at least one from the group of an oxidative stress condition, a neurodegeneration condition, a neuronal damage condition, and an abnormal metabolic condition within the neural target; and
(d) applying optical signals to the neural target.

47. The method of claim 46 wherein the step of (d) occurs in the event that at least one from the group of an oxidative stress condition, a neurodegeneration condition, a neuronal damage condition, and an abnormal metabolic condition is likely or expected to exist within the neural target.

48. The method of claim 46 wherein the step of (b) occurs in the event that the first set of sensed parameters corresponds to at least one from the group of an oxidative stress condition, a neurodegeneration condition, a neuronal damage condition, and an abnormal metabolic condition within the neural target.

49. The method of claim 46 further comprising determining whether the first set of sensed parameters and the second sets of sensed parameters correspond to at least one from the group of an oxidative stress condition, a neurodegeneration condition, a neuronal damage condition and an abnormal metabolic condition within the neural target.

50. The method of claim 46 wherein the first set of sensing devices and the second set of sensing devices is different from one another.

51. The method of claim 46 wherein the first set of sensing devices is implanted in a patient.

52. The method of claim 46 wherein the first set of sensing devices is implanted in a patient, and wherein applying optical signals to the neural target is performed using a device implanted in the patient.

53. The method of claim 46 wherein the steps of (a), (c) and (d) are performed automatically by a set of devices implanted in a patient.

54. A neuroillumination method comprising:
measuring a bodily fluid pressure parameter;
determining whether the bodily fluid pressure parameter exceeds a reference fluid pressure value; and
applying optical signals to a neural target.

55. The method of claim 54 wherein the optical signals are applied at a wavelength ranging from about 670 nm to about 820 nm.

56. The method of claim 54 wherein applying optical signals to the neural target occurs in the event that the fluid pressure parameter exceeds the reference fluid pressure value.

57. The method of claim 54 wherein determining whether the bodily fluid pressure parameter exceeds the reference fluid pressure value is performed by an implanted device.

58. The method of claim 54 wherein measuring the bodily fluid pressure parameter is performed using a device implanted in a cerebral ventricle.

59. The method of claim 54 wherein measuring the bodily fluid pressure parameter and applying optical signals to the neural target are performed by a set of implanted devices.

60. The method of claim 54 wherein applying optical signals to the neural target is performed using an illumination probe comprising an elongate member and a light emission site carried by the elongate member.

61. The method of claim 60 wherein a portion of the illumination probe is carried by a portion of a shunt catheter.

62. A method for treating a neural condition of a patient comprising:
implanting an illumination probe into a cerebral ventricle of the patient, the illumination probe operable to emit near-infrared light; and
preferentially directing near-infrared light emitted by the illumination probe toward a predetermined set of neural targets.

63. The method of claim 62 wherein the predetermined set of neural targets includes at least one neural target selected from the group consisting of the nucleus accumbens, the orbitofrontal cortex, the prefrontal cortex, the parietal lobe, the cingulate gyrus, the hypothalamus, the pineal region, the midbrain, the basal ganglia, the substantia nigra, the thalamus, the subthalamus, the nucleus of Meynert, the optic tract, the optic nerve, the hippocampus, the amygdala, the amygdaloid body, the caudate nucleus, Brodmann area 9, Brodmann area 10, Brodmann area 25, and Brodmann area 46

64. A tissue illumination method comprising:
implanting an illumination shunt system into a patient, the illumination shunt system including:
a ventricular shunt apparatus;
a tissue illumination apparatus; and
a telemetry circuit coupled to at least one of the ventricular shunt apparatus and the tissue illumination apparatus;
establishing a wireless communication link between the telemetry circuit and a programming device; and
wirelessly communicating one from the group of a shunt control parameter and a tissue illumination parameter from the programming device to the telemetry circuit.

65. The method of claim 64 further comprising illuminating neural tissue with near-infrared light.

66. The method of claim 64 further comprising preferentially applying illumination to a predetermined set of neural targets, the illumination applied at a wavelength ranging from about 670 nm to about 820 nm.
